(19) United States
(12) Patent Application Publication
(10) Pub. No.: US 2026/0217734 A1
(43) Pub. Date: Jul. 30, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Doo-Hyeon MOON, Gyeonggi-do (KR); Tae-Jin LEE, Gyeonggi-do (KR); Dong-Hyung LEE, Gyeonggi-do (KR); So-Mi PARK, Gyeonggi-do (KR); Du-Yong PARK, Gyeonggi-do (KR)

(21) Appl. No.: 19/577,924

(22) Filed: Mar. 25, 2026

Related U.S. Application Data
(63) Continuation of application No. 17/423,767, filed on Jul. 16, 2021, now abandoned.

(30) Foreign Application Priority Data
Jan. 18, 2019 (KR) 10-2019-0006615
Nov. 14, 2019 (KR) 10-2019-0145630

(51) Int. Cl. C07D 519/00; C07D 209/86; H10K 50/11; H10K 50/15; H10K 85/60

(52) U.S. Cl. CPC C07D 519/00; C07D 209/86; H10K 85/624; H10K 85/626; H10K 85/633; H10K 85/636; H10K 85/6572; H10K 85/6574; H10K 85/6576; H10K 50/11; H10K 50/15

(57) ABSTRACT

The present disclosure relates to an organic electroluminescent compound and an organic electroluminescent device comprising the same. By comprising the organic electroluminescent compound according to the present disclosure, an organic electroluminescent device having improved luminous efficiency can be provided.

### CLAIM OF BENEFIT OF PRIOR APPLICATION

This application is a continuation of U.S. patent application Ser. No. 17/423,767, filed Jul. 16, 2021, which is the National Stage Entry of PCT/KR2019/016828, filed Dec. 2, 2019, both of which are incorporated by reference herein in their entirety.

### TECHNICAL FIELD

The present disclosure relates to an organic electroluminescent compound and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic EL device was developed by Eastman Kodak in 1987, by using small aromatic diamine molecules and aluminum complexes as materials for forming a light-emitting layer [Appl. Phys. Lett. 51, 913, 1987].

An organic EL device (OLED) changes electric energy into light by the injection of a charge into an organic light-emitting material, and commonly comprises an anode, a cathode, and an organic layer formed between the two electrodes. The organic layer of the organic EL device may be composed of a hole injection layer, a hole transport layer, a hole auxiliary layer, a light-emitting auxiliary layer, an electron blocking layer, a light-emitting layer (containing host and dopant materials), an electron buffer layer, a hole blocking layer, an electron transport layer, an electron injection layer, etc. The materials used in the organic layer can be classified into a hole injection material, a hole transport material, a hole auxiliary material, a light-emitting auxiliary material, an electron blocking material, a light-emitting material, an electron buffer material, a hole blocking material, an electron transport material, an electron injection material, etc., depending on functions. In the organic EL device, holes from an anode and electrons from a cathode are injected into a light-emitting layer by applying voltage, and an exciton having high energy is produced by the recombination of the holes and electrons. The organic light-emitting compound moves into an excited state by the energy and emits light from energy when the organic light-emitting compound returns to the ground state from the excited state.

The important factor determining luminous efficiency in an organic EL device is light-emitting materials. The light-emitting materials are required to have high quantum efficiency, high movement degree of an electron and a hole, and uniformity and stability of the formed light-emitting material layer. Such light-emitting materials are classified into blue, green, and red light-emitting materials according to the light-emitting color, and further include yellow or orange light-emitting materials. In addition, the light-emitting materials are classified into a host material and a dopant material in a functional aspect. Recently, an urgent task is the development of an organic EL device having high efficiency and long lifespan. In particular, the development of highly excellent light-emitting material over conventional materials is urgently required, considering the EL properties necessary for medium- and large-sized OLED panels. For this, the desirable properties of the host material, which acts as a solvent and the sole energy transporter in the solid state, should be high purity and have a suitable molecular weight to enable vacuum deposition. Furthermore, a host material is required to have high glass transition temperature and pyrolysis temperature to achieve thermal stability, high electrochemical stability to achieve long lifespan, easy formability of an amorphous thin film, good adhesion with adjacent layers, and no movement between layers.

In addition, development of materials having good thermal stability in a hole transport layer, a buffer layer, an electron transport layer, etc., and capable of improving the performance of an organic electroluminescent device, such as driving voltage, luminescent efficiency, and lifespan, is required.

JP 5,609,256 B2 discloses 2-aminocarbazole compound suitable for a hole transport material of the organic EL device; however, it does not disclose a compound substituted with an amino compound at 1-position of carbazole.

### DISCLOSURE OF INVENTION

### Technical Problem

The object of the present disclosure is to provide an organic electroluminescent compound capable of firstly producing an organic electroluminescent device having improved luminous efficiency, and secondly, to provide the organic electroluminescent device comprising the organic electroluminescent compound.

### Solution to Problem

The present inventors found that an organic electroluminescent device can exhibit improved luminous efficiency by comprising the specific compound containing a structure in which an amino compound is bonded at the 1-position of carbazole in a hole transport layer and/or a light-emitting layer, so that the present invention was completed. Specifically, the aforementioned objective can be achieved by the organic electroluminescent compound represented by the following formula 1, so that the present invention was completed.

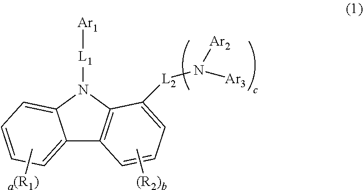

In formula 1,
Ar₁ to Ar₃ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
L₁ and L₂ each independently represent a single bond, a substituted or unsubstituted (C6-C30)arylene, or a substituted or unsubstituted (3- to 30-membered)heteroarylene;
R₁ and R₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
a represents an integer of 1 to 4, b represents an integer of 1 to 3, c represents an integer of 1 or 2, provided that when L₂ is a single bond, c is 1; and
when a, b, and c are 2 or more, each of R₁, each of R₂, and each of —NAr₂ Ar₃ may be the same or different.

### Advantageous Effects of Invention

The organic electroluminescent device having improved luminous efficiency can be prepared, by comprising an organic electroluminescent compound according to the present disclosure.

### MODE FOR THE INVENTION

Hereinafter, the present disclosure will be described in detail. However, the following description is intended to explain the invention, and is not meant in any way to restrict the scope of the invention.

The present disclosure relates to an organic electroluminescent compound represented by formula 1. More specifically, the present disclosure relates to an organic electroluminescent material comprising the organic electroluminescent compound, and an organic electroluminescent device comprising the organic electroluminescent compound.

The term “organic electroluminescent compound” in the present disclosure means a compound that may be used in an organic electroluminescent device, and may be comprised in any material layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. “(C3-C30)cycloalkyl” is a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, etc.

Herein, “(3- to 30-membered)heteroaryl(ene)” is an aryl having 3 to 30 ring backbone atoms, in which the number of ring backbone atoms is preferably 5 to 25, including at least one, preferably 1 to 4 heteroatoms selected from the group consisting of B, N, O, S, Si, P, and Ge. The above heteroaryl may be a monocyclic ring, or a fused ring condensed with at least one benzene ring; and may be partially saturated. Also, the above heteroaryl may be one formed by linking at least one heteroaryl or aryl group to a heteroaryl group via a single bond(s); and may comprise a spiro structure. Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acrylidinyl, silafluorenyl, germafluorenyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazole-1-yl, azacarbazole-2-yl, azacarbazole-3-yl, azacarbazole-4-yl, azacarbazole-5-yl, azacarbazole-6-yl, azacarbazole-7-yl, azacarbazole-8-yl, azacarbazole-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acrylidinyl, 2-acrylidinyl, 3-acrylidinyl, 4-acrylidinyl, 9-acrylidinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrole-1-yl, 2-methylpyrrole-3-yl, 2-methylpyrrole-4-yl, 2-methylpyrrole-5-yl, 3-methylpyrrole-1-yl, 3-methylpyrrole-2-yl, 3-methylpyrrole-4-yl, 3-methylpyrrole-5-yl, 2-t-butylpyrrole-4-yl, 3-(2-phenylpropyl)pyrrole-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc.

Herein, “halogen” includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents; preferably, may be a substituted or unsubstituted (3- to 26-membered) mono- or polycyclic, alicyclic, aromatic ring, or a combination thereof. In addition, at least one of the carbon atoms in the formed ring may be replaced with at least one heteroatom selected from the group consisting of B, N, O, S, Si, and P, preferably, N, O, and S. According to one embodiment, the number of atoms in the ring skeleton is 5 to 20, according to another embodiment, the number of atoms in the ring skeleton is 5 to 15. In one embodiment, the fused ring may be, e.g., a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent. The substituents of the substituted (C1-C30)alkyl, the substituted (C6-C30)aryl(ene), the substituted (3- to 30-membered)heteroaryl(ene), the substituted (C3-C30)cycloalkyl, the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino and the substituted (C1-C30)alkyl(C6-C30)arylamino in Ar₁ to Ar₃, L₁ to L₃, R₁, and R₂ are each independently at least one selected from the group consisting of deuterium, halogen, cyano, carboxyl, nitro, hydroxyl, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (C6-C30)aryl-substituted or unsubstituted (5- to 30-membered)heteroaryl, (5- to 30-membered)heteroaryl-substituted or unsubstituted (C6-C30)aryl, tri(C1-C30)alkylsilyl, tri(C6-C30)arylsilyl, di(C1-C30)alkyl(C6-C30)arylsilyl, (C1-C30)alkyldi(C6-C30)arylsilyl, amino, mono- or di-(C1-C30)alkylamino, (C1-C30)alkyl-substituted or unsubstituted mono- or di-(C6-C30)arylamino, (C1-C30)alkyl(C6-C30)arylamino, (C1-C30)alkylcarbonyl, (C1-C30)alkoxycarbonyl, (C6-C30)arylcarbonyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl. For example, the substituents may be an unsubstituted methyl, an unsubstituted phenyl, an unsubstituted biphenyl, or an unsubstituted naphthyl, etc.

Hereinafter, the organic electroluminescent compound according to one embodiment will be described.

The organic electroluminescent compound according to one embodiment is represented by the following formula 1.

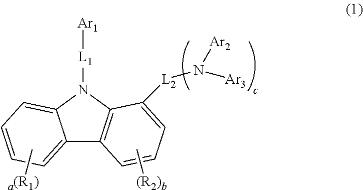

In formula 1,
Ar₁ to Ar₃ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
L₁ and L₂ each independently represent a single bond, a substituted or unsubstituted (C6-C30)arylene, or a substituted or unsubstituted (3- to 30-membered)heteroarylene;
R₁ and R₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
a represents an integer of 1 to 4, b represents an integer of 1 to 3, c represents an integer of 1 or 2, provided that when L₂ is a single bond, c is 1; and
when a, b, and c are 2 or more, each of R₁, each of R₂, and each of —NAr₂ Ar₃ may be the same or different.

The organic electroluminescent compound of formula 1 according to one embodiment may be represented by the following formula 2 or 3:

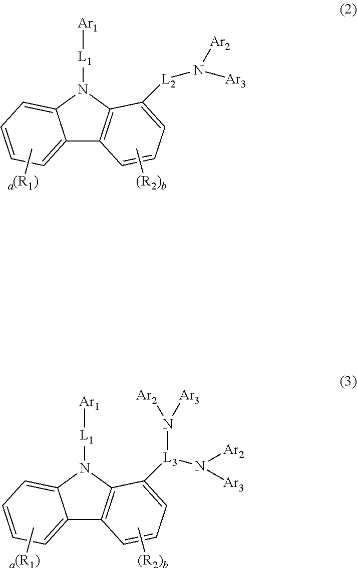

In formulae 2 and 3,
Ar₁ to Ar₃, L₁, L₂, R₁ and R₂, a, and b are as defined in formula 1; and
L₃ represents a substituted or unsubstituted (C6-C30)arylene or a substituted or unsubstituted (3- to 30-membered)heteroarylene.

In one embodiment, L₁ represents a single bond, a substituted or unsubstituted (C6-C30)arylene, or a substituted or unsubstituted (3- to 30-membered)heteroarylene, preferably, may be a single bond or a substituted or unsubstituted (C6-C25)arylene, more preferably, a single bond or a substituted or unsubstituted (C6-C18)arylene. For example, L₁ may be a single bond, a substituted or unsubstituted phenylene, a substituted or unsubstituted biphenylene, or a substituted or unsubstituted naphthylene.

In one embodiment, Ar₁ represents hydrogen, deuterium, a substituted or unsubstituted (C1-C30)alkyl, a substituted or unsubstituted (C6-C30)aryl, a substituted or unsubstituted (3 to 30-membered)heteroaryl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino, preferably, may be hydrogen, deuterium, or a substituted or unsubstituted (C6-C25)aryl, more preferably, a substituted or unsubstituted (C6-C18)aryl. For example, Ar₁ may be a substituted or unsubstituted phenyl, a substituted or unsubstituted o-biphenyl, a substituted or unsubstituted m-biphenyl, a substituted or unsubstituted p-biphenyl, a substituted or unsubstituted naphthyl, a substituted or unsubstituted p-terphenyl, or a substituted or unsubstituted m-terphenyl.

In one embodiment, L₂ represents a single bond, a substituted or unsubstituted (C6-C30)arylene, or a substituted or unsubstituted (3- to 30-membered)heteroarylene, preferably, may be a single bond or a substituted or unsubstituted (C6-C25)arylene, more preferably, a single bond or a substituted or unsubstituted (C6-C18)arylene. For example, L₂ may be a single bond, a substituted or unsubstituted phenylene, a substituted or unsubstituted p-biphenylene, a substituted or unsubstituted m-biphenylene, a substituted or unsubstituted naphthylene, a substituted or unsubstituted phenylnaphthylene, or a substituted or unsubstituted naphthylphenylene.

In one embodiment, L₃ may be a substituted or unsubstituted (C6-C30)arylene or a substituted or unsubstituted (3- to 30-membered)heteroarylene, preferably, a substituted or unsubstituted (C6-C25)arylene, more preferably, a substituted or unsubstituted (C6-C18)arylene. For example, L₃ may be a substituted or unsubstituted phenylene.

In one embodiment, Ar₂, and Ar₃ represent hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino, preferably, may be hydrogen, deuterium, a substituted or unsubstituted (C6-C30)aryl, or a substituted or unsubstituted (3 to 25-membered)heteroaryl, more preferably, a substituted or unsubstituted (C6-C25)aryl or a substituted or unsubstituted (5- to 25-membered)heteroaryl.

In one embodiment, Ar₂ and Ar₃ each independently may be selected from any one of the substituents listed in the following Group 1.

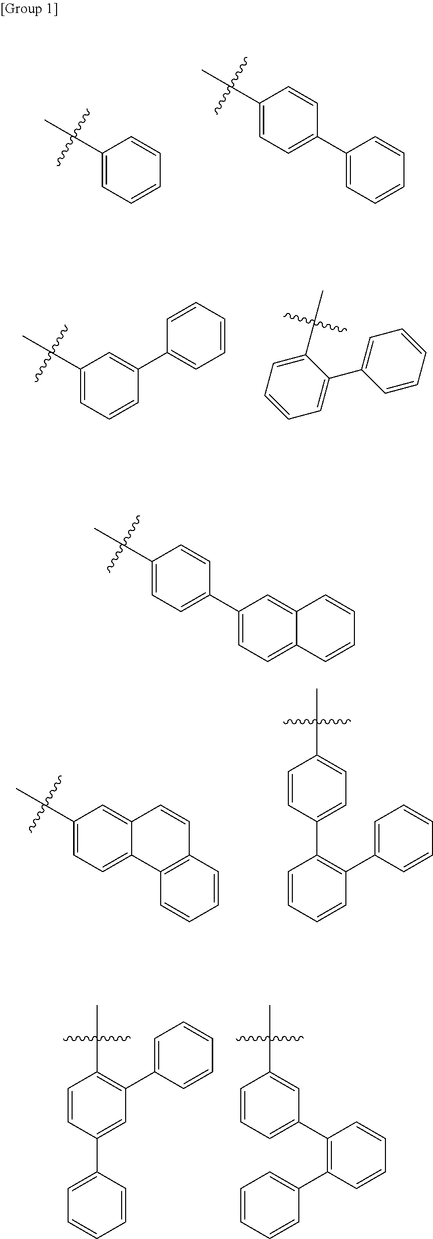

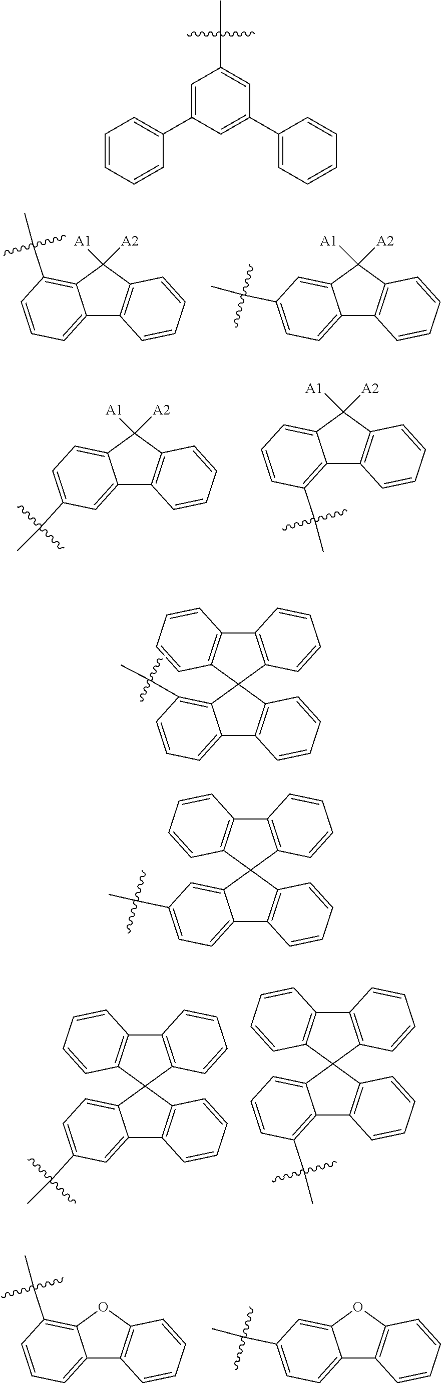

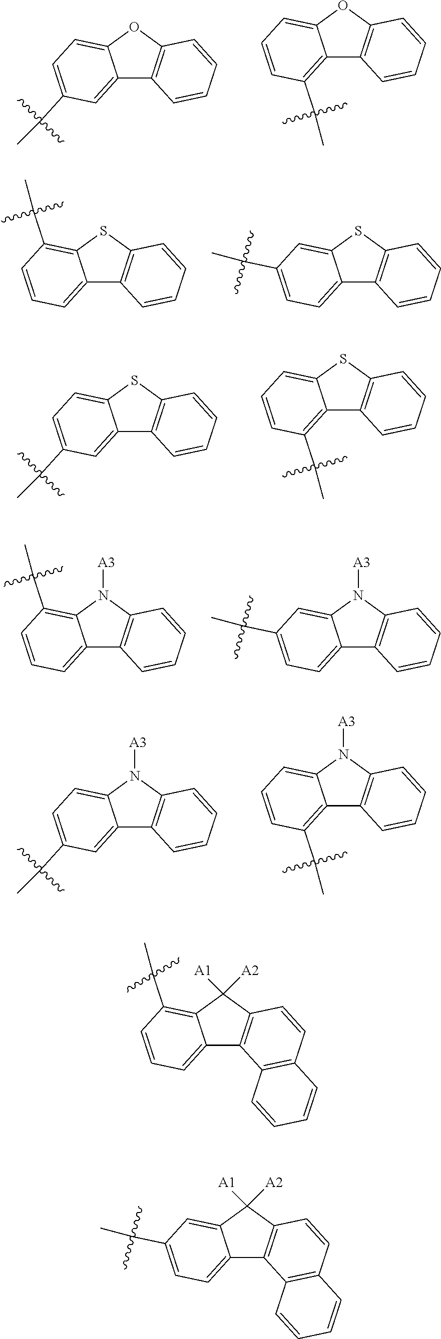

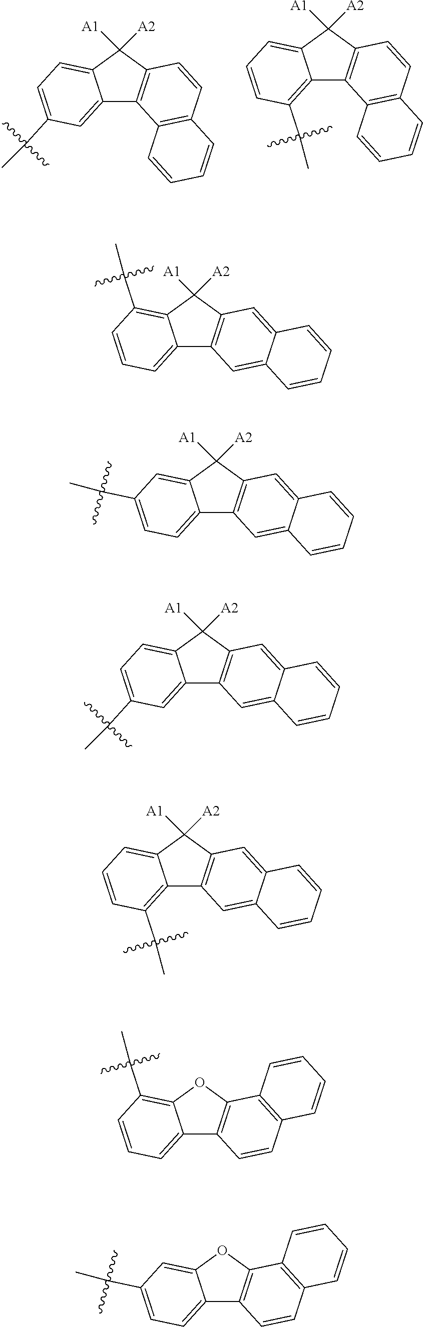

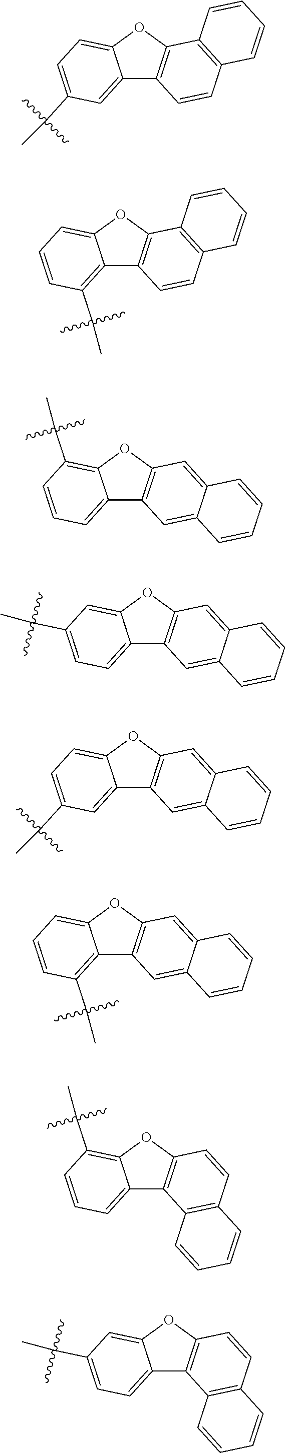

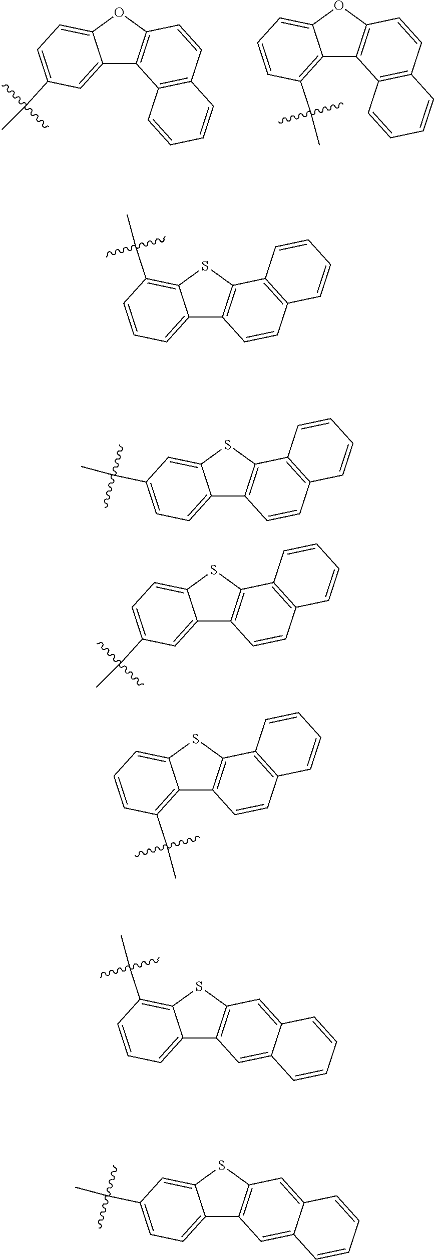

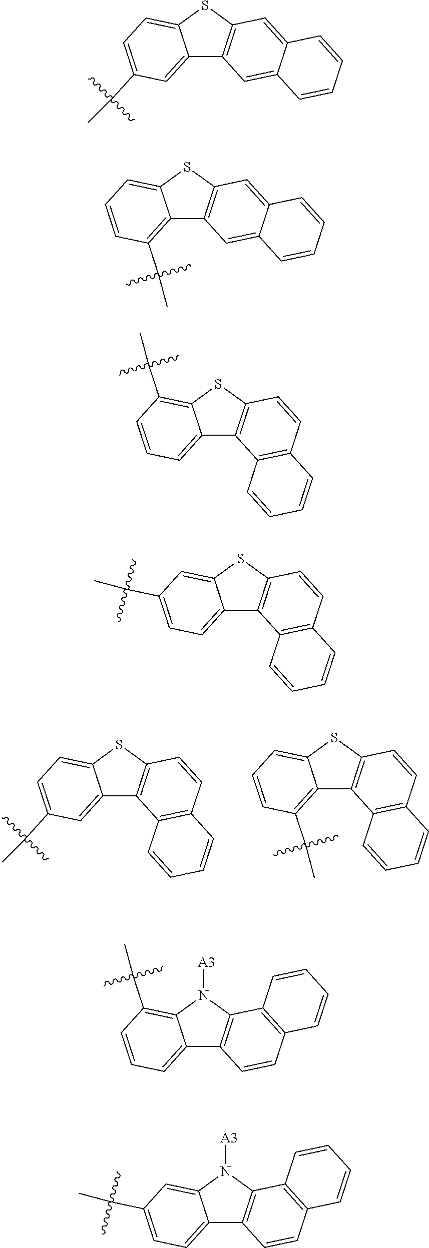

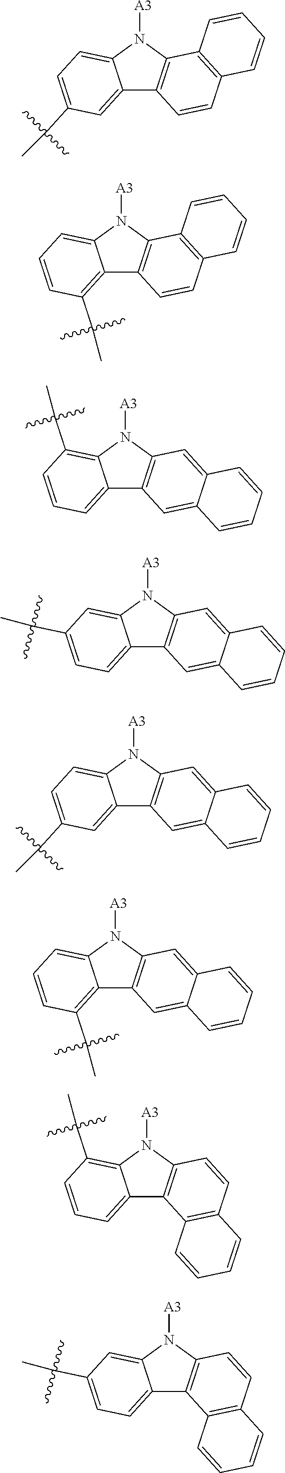

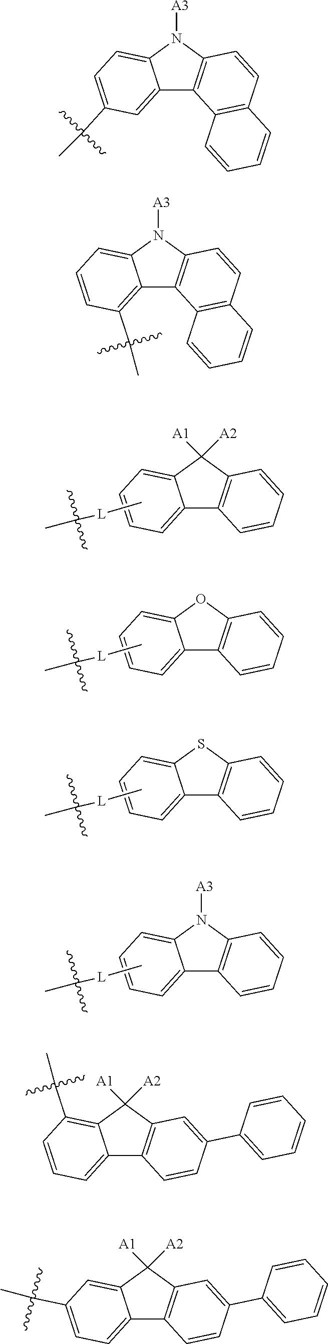

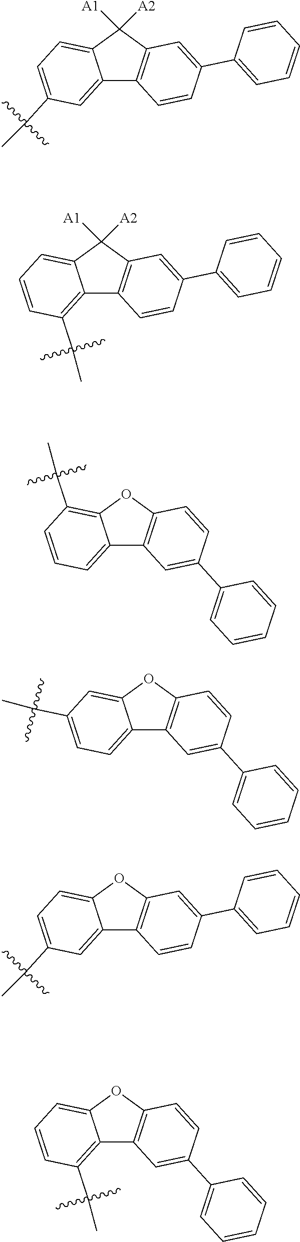

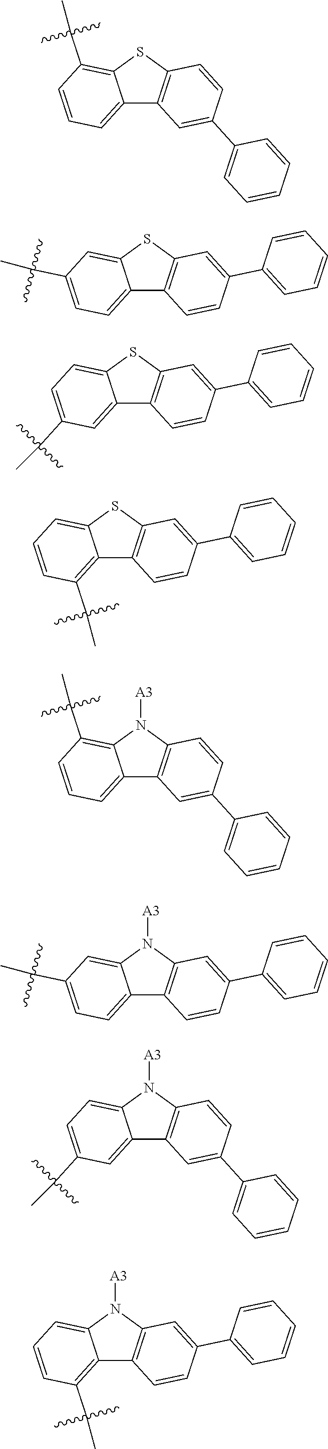

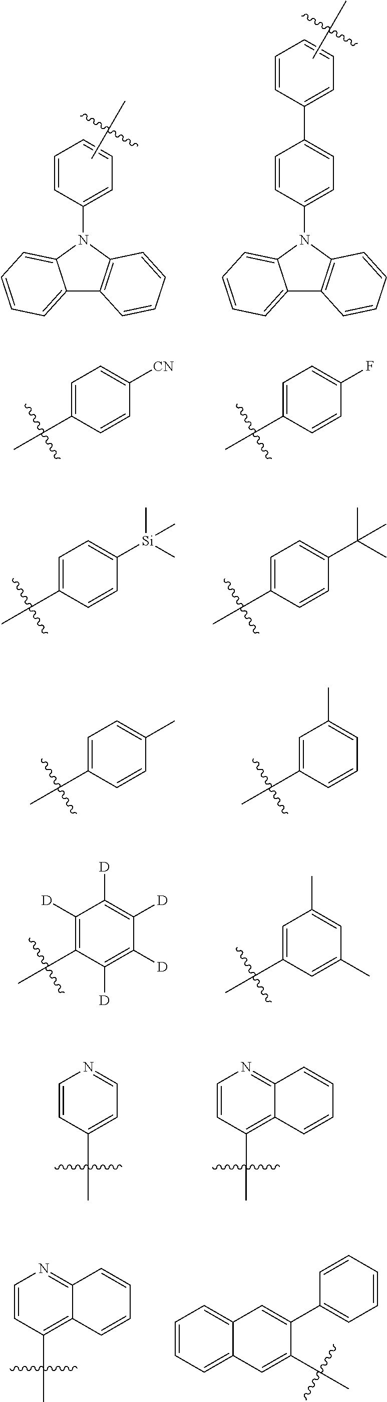

In Group 1,
A1 to A3 each independently represent a substituted or unsubstituted (C1-C30)alkyl, a substituted or unsubstituted (C6-C30)aryl, or a substituted or unsubstituted (3- to 30-membered)heteroaryl; and
L represents a substituted or unsubstituted (C6-C30)arylene or a substituted or unsubstituted (3- to 30-membered)heteroarylene.

In Group 1, preferably, A1 and A2 each independently may be a substituted or unsubstituted (C1-C10)alkyl or a substituted or unsubstituted (C6-C18)aryl, more preferably, a substituted or unsubstituted (C1-C6)alkyl or a substituted or unsubstituted (C6-C12)aryl.

In Group 1, preferably, A3 may be a substituted or unsubstituted (C6-C25)aryl, more preferably, a substituted or unsubstituted (C6-C18)aryl.

For example, Ar₂ and Ar₃ each independently may be a substituted or unsubstituted phenyl, a substituted or unsubstituted o-biphenyl, a substituted or unsubstituted m-biphenyl, a substituted or unsubstituted p-biphenyl, a substituted or unsubstituted fluorenyl, a substituted or unsubstituted spirobifluorenyl, a substituted or unsubstituted p-terphenyl, a substituted or unsubstituted carbazolyl, a substituted or unsubstituted dibenzofuranyl, or a substituted or unsubstituted dibenzothiophenyl.

In one embodiment, R₁ and R₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino, preferably, may be hydrogen, deuterium, halogen, cyano, or a substituted or unsubstituted (C6-C25)aryl, more preferably, hydrogen, deuterium, or a substituted or unsubstituted (C6-C18)aryl. For example, R₁ and R₂ each independently may be hydrogen or a substituted or unsubstituted phenyl.

According to one embodiment, the organic electroluminescent compound of formula 1 may be in that Ar₁ to Ar₃ each independently represent a substituted or unsubstituted (C6-C30)aryl or a substituted or unsubstituted (5- to 25-membered)heteroaryl; L₁ and L₂ each independently represent a single bond, a substituted or unsubstituted (C6-C25)arylene, or a substituted or unsubstituted (5- to 15-membered)heteroarylene; and R₁ and R₂ each independently represent hydrogen, a substituted or unsubstituted (C1-C6)alkyl, or a substituted or unsubstituted (C6-C12)aryl.

According to one embodiment, the organic electroluminescent compound of formula 1 may be in that Ar₁ to Ar₃ each independently represent a (C6-C30)aryl unsubstituted or substituted with at least one of (C1-C6)alkyl and (C6-C12)aryl, or a (C6-C12)aryl-substituted or unsubstituted (5- to 20-membered)heteroaryl; L₁ and L₂ each independently represent a single bond or an unsubstituted (C6-C18)arylene; and R₁ and R₂ each independently represent hydrogen or an unsubstituted (C6-C12)aryl.

According to one embodiment, the compound represented by formula 1 may be more specifically illustrated by the following compounds, but is not limited thereto.

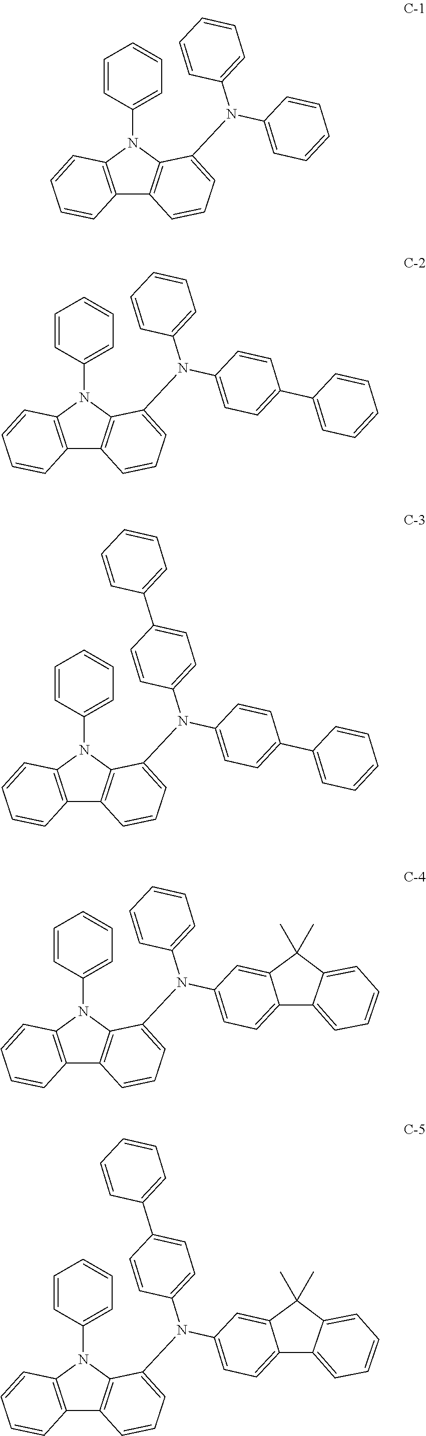 [SMILES: c1ccc(N(c2ccccc2)c2cccc3c4ccccc4n(-c4ccccc4)c23)cc1 ; c1ccc(-c2ccc(N(c3ccccc3)c3cccc4c5ccccc5n(-c5ccccc5)c34)cc2)cc1 ; c1ccc(-c2ccc(N(c3ccc(-c4ccccc4)cc3)c3cccc4c5ccccc5n(-c5ccccc5)c34)cc2)cc1 ; CC1(C)c2ccccc2-c2ccc(N(c3ccccc3)c3cccc4c5ccccc5n(-c5ccccc5)c34)cc21 ; CC1(C)c2ccccc2-c2ccc(N(c3ccc(-c4ccccc4)cc3)c3cccc4c5ccccc5n(-c5ccccc5)c34)cc21]

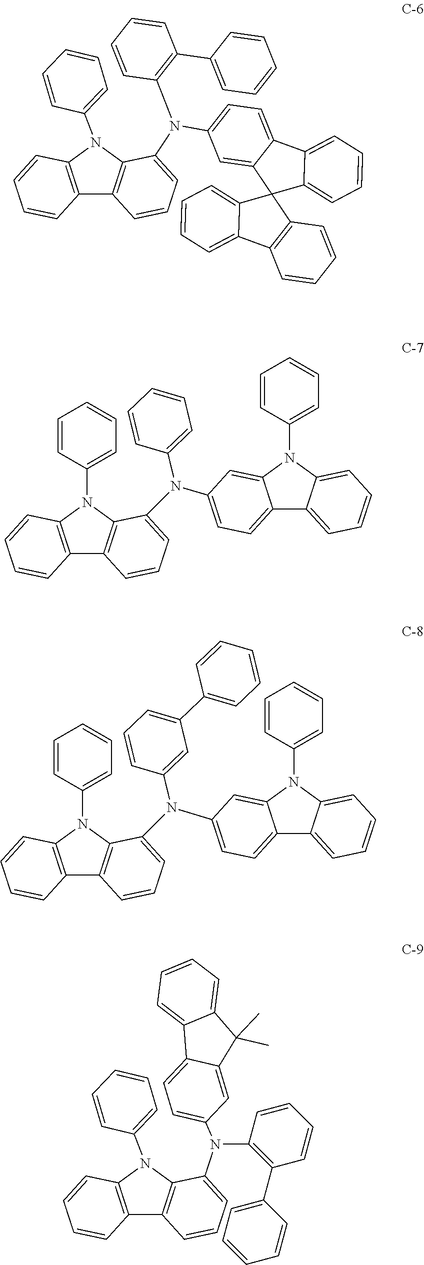 [SMILES: c1ccc(-c2ccccc2N(c2ccc3c(c2)C2(c4ccccc4-c4ccccc42)c2ccccc2-3)c2cccc3c4ccccc4n(-c4ccccc4)c23)cc1 ; c1ccc(N(c2ccc3c4ccccc4n(-c4ccccc4)c3c2)c2cccc3c4ccccc4n(-c4ccccc4)c23)cc1 ; c1ccc(-c2cccc(N(c3ccc4c5ccccc5n(-c5ccccc5)c4c3)c3cccc4c5ccccc5n(-c5ccccc5)c34)c2)cc1 ; CC1(C)c2ccccc2-c2ccc(N(c3ccccc3-c3ccccc3)c3cccc4c5ccccc5n(-c5ccccc5)c34)cc21]

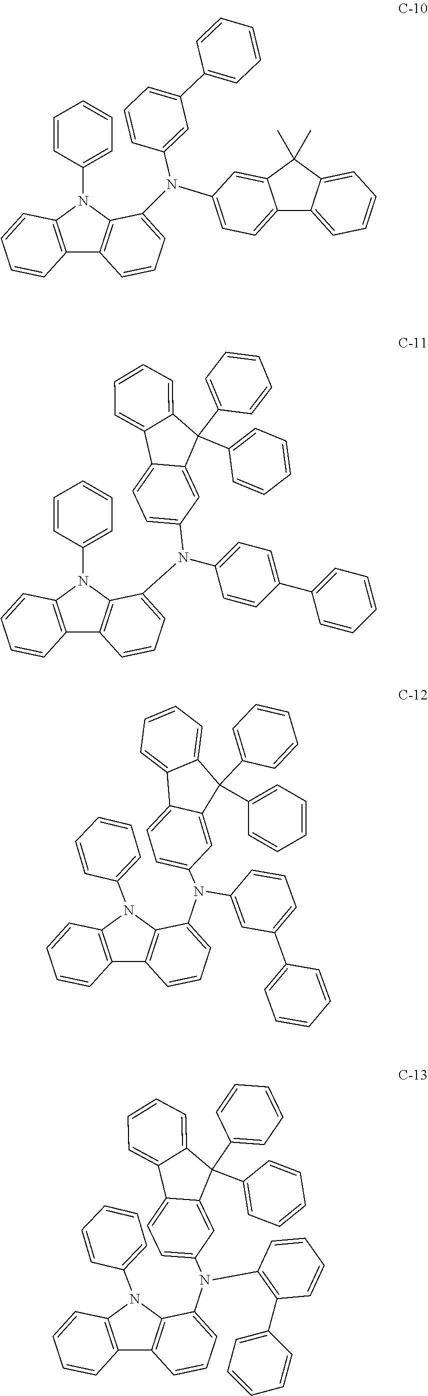 [SMILES: CC1(C)c2ccccc2-c2ccc(N(c3cccc(-c4ccccc4)c3)c3cccc4c5ccccc5n(-c5ccccc5)c34)cc21 ; c1ccc(-c2ccc(N(c3ccc4c(c3)C(c3ccccc3)(c3ccccc3)c3ccccc3-4)c3cccc4c5ccccc5n(-c5ccccc5)c34)cc2)cc1 ; c1ccc(-c2cccc(N(c3ccc4c(c3)C(c3ccccc3)(c3ccccc3)c3ccccc3-4)c3cccc4c5ccccc5n(-c5ccccc5)c34)c2)cc1 ; c1ccc(-c2ccccc2N(c2ccc3c(c2)C(c2ccccc2)(c2ccccc2)c2ccccc2-3)c2cccc3c4ccccc4n(-c4ccccc4)c23)cc1]

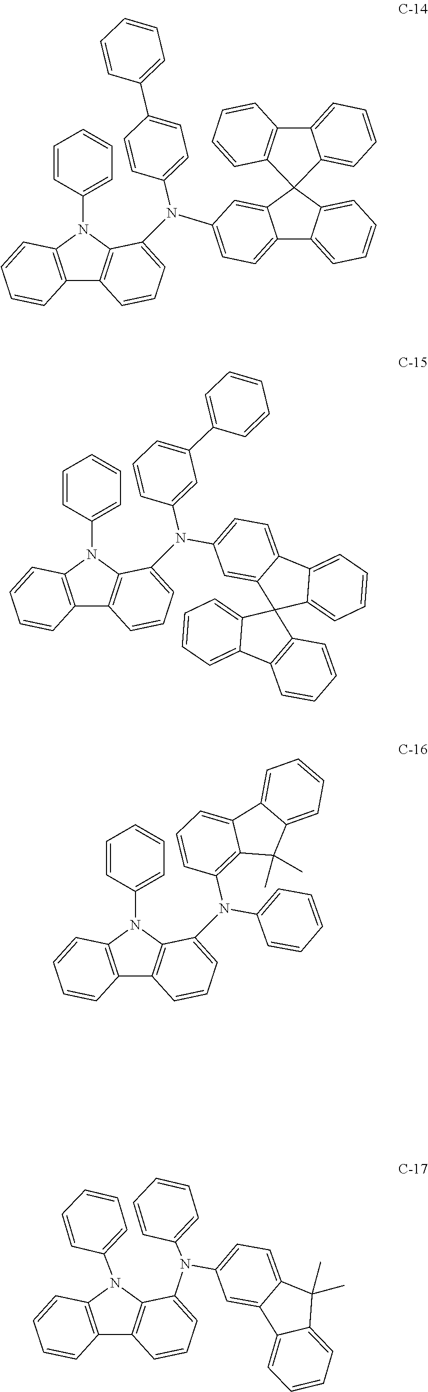 [SMILES: c1ccc(-c2ccc(N(c3ccc4c(c3)C3(c5ccccc5-c5ccccc53)c3ccccc3-4)c3cccc4c5ccccc5n(-c5ccccc5)c34)cc2)cc1 ; c1ccc(-c2cccc(N(c3ccc4c(c3)C3(c5ccccc5-c5ccccc53)c3ccccc3-4)c3cccc4c5ccccc5n(-c5ccccc5)c34)c2)cc1 ; CC1(C)c2ccccc2-c2cccc(N(c3ccccc3)c3cccc4c5ccccc5n(-c5ccccc5)c34)c21 ; CC1(C)c2ccccc2-c2cc(N(c3ccccc3)c3cccc4c5ccccc5n(-c5ccccc5)c34)ccc21]

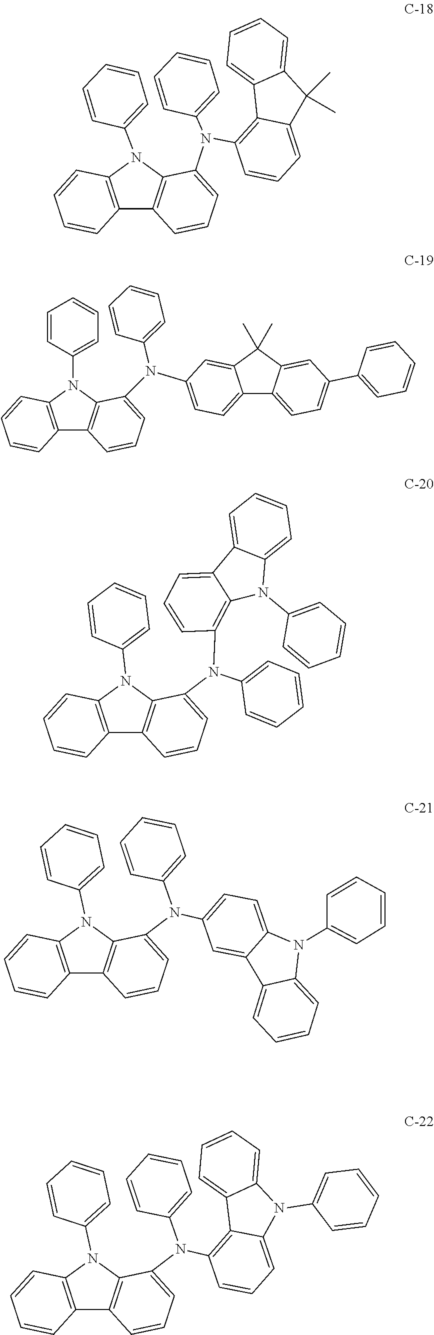 [SMILES: CC1(C)c2ccccc2-c2c(N(c3ccccc3)c3cccc4c5ccccc5n(-c5ccccc5)c34)cccc21 ; CC1(C)c2cc(-c3ccccc3)ccc2-c2ccc(N(c3ccccc3)c3cccc4c5ccccc5n(-c5ccccc5)c34)cc21 ; c1ccc(N(c2cccc3c4ccccc4n(-c4ccccc4)c23)c2cccc3c4ccccc4n(-c4ccccc4)c23)cc1 ; c1ccc(N(c2ccc3c(c2)c2ccccc2n3-c2ccccc2)c2cccc3c4ccccc4n(-c4ccccc4)c23)cc1 ; c1ccc(N(c2cccc3c2c2ccccc2n3-c2ccccc2)c2cccc3c4ccccc4n(-c4ccccc4)c23)cc1]

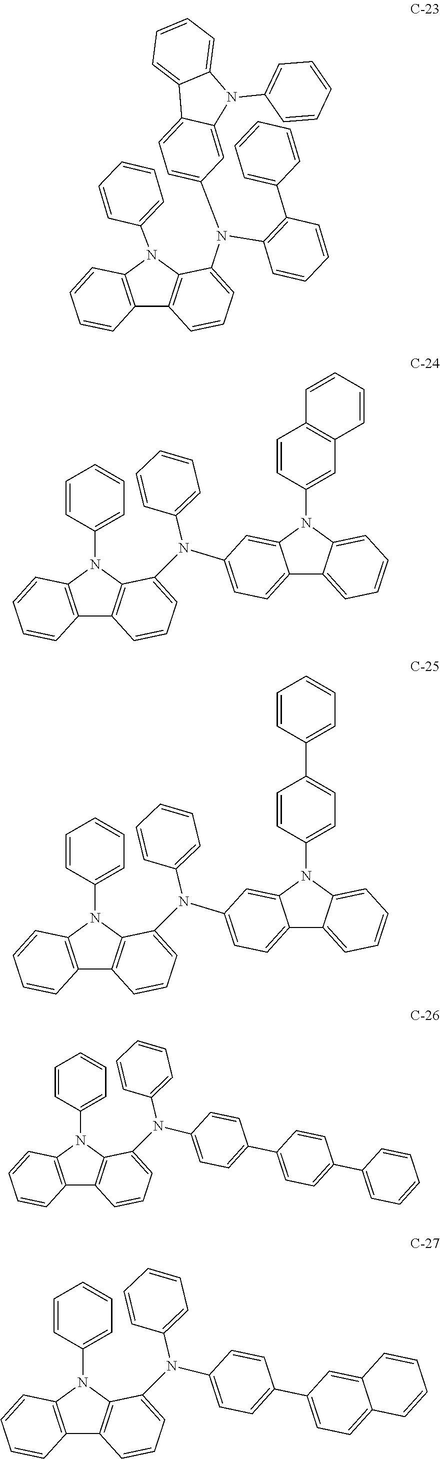 [SMILES: c1ccc(-c2ccccc2N(c2ccc3c4ccccc4n(-c4ccccc4)c3c2)c2cccc3c4ccccc4n(-c4ccccc4)c23)cc1 ; c1ccc(N(c2ccc3c4ccccc4n(-c4ccc5ccccc5c4)c3c2)c2cccc3c4ccccc4n(-c4ccccc4)c23)cc1 ; c1ccc(-c2ccc(-n3c4ccccc4c4ccc(N(c5ccccc5)c5cccc6c7ccccc7n(-c7ccccc7)c56)cc43)cc2)cc1 ; c1ccc(-c2ccc(-c3ccc(N(c4ccccc4)c4cccc5c6ccccc6n(-c6ccccc6)c45)cc3)cc2)cc1 ; c1ccc(N(c2ccc(-c3ccc4ccccc4c3)cc2)c2cccc3c4ccccc4n(-c4ccccc4)c23)cc1]

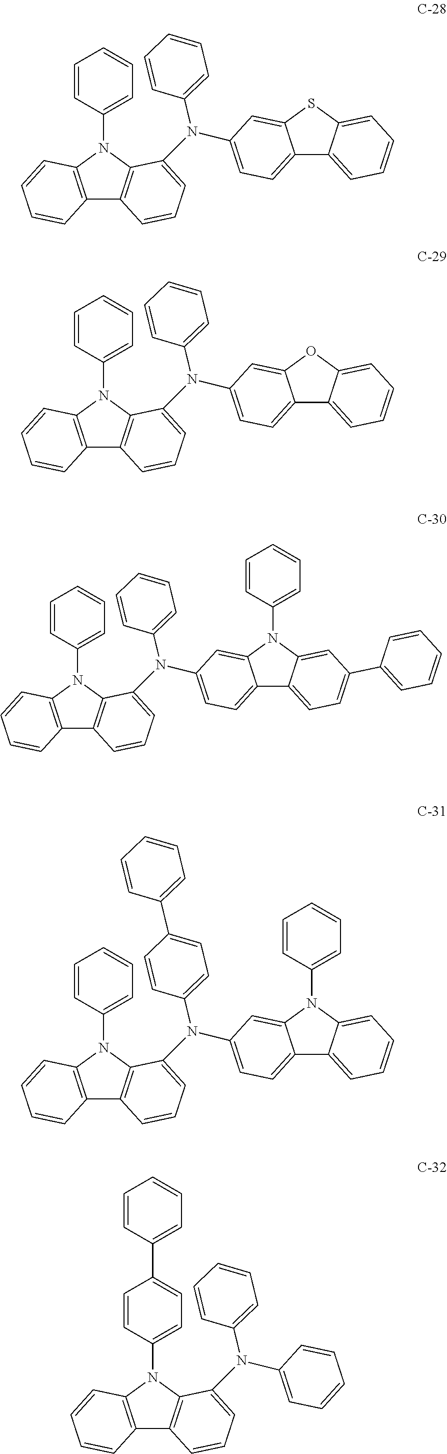 [SMILES: c1ccc(N(c2ccc3c(c2)sc2ccccc23)c2cccc3c4ccccc4n(-c4ccccc4)c23)cc1 ; c1ccc(N(c2ccc3c(c2)oc2ccccc23)c2cccc3c4ccccc4n(-c4ccccc4)c23)cc1 ; c1ccc(-c2ccc3c4ccc(N(c5ccccc5)c5cccc6c7ccccc7n(-c7ccccc7)c56)cc4n(-c4ccccc4)c3c2)cc1 ; c1ccc(-c2ccc(N(c3ccc4c5ccccc5n(-c5ccccc5)c4c3)c3cccc4c5ccccc5n(-c5ccccc5)c34)cc2)cc1 ; c1ccc(-c2ccc(-n3c4ccccc4c4cccc(N(c5ccccc5)c5ccccc5)c43)cc2)cc1]

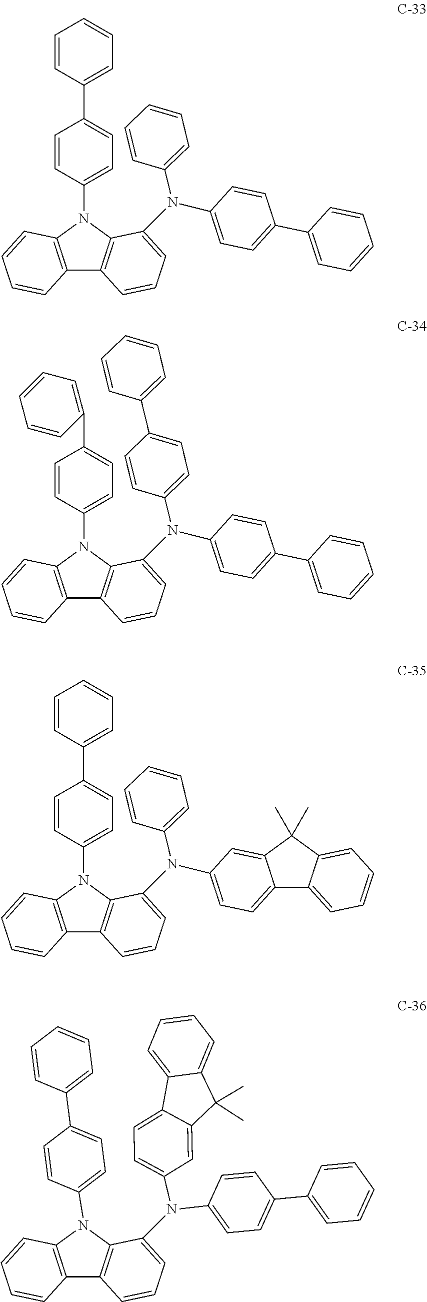

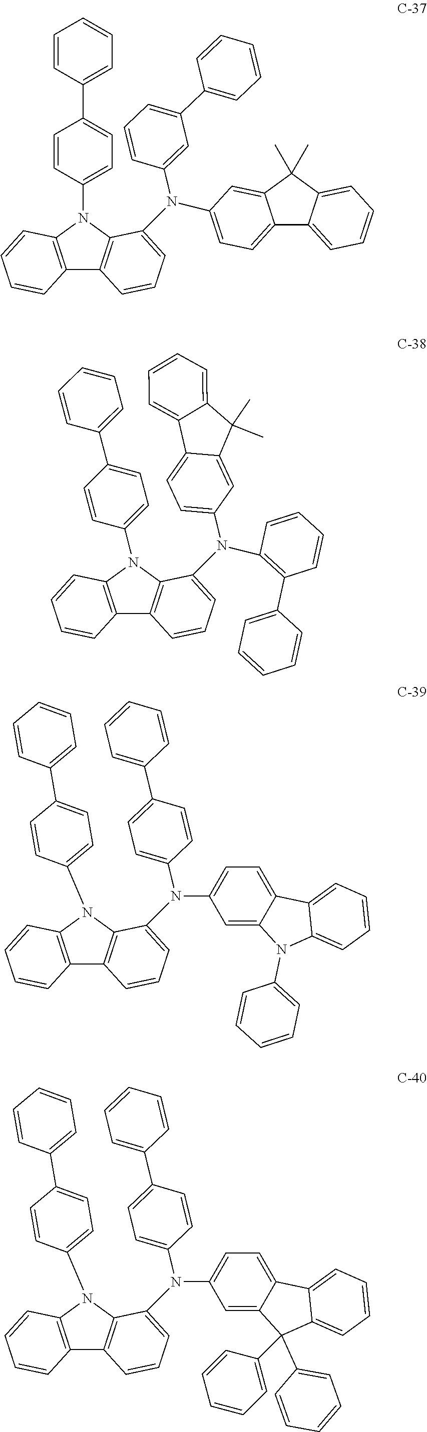 [SMILES: CC1(C)c2ccccc2-c2ccc(N(c3cccc(-c4ccccc4)c3)c3cccc4c5ccccc5n(-c5ccc(-c6ccccc6)cc5)c34)cc21 ; CC1(C)c2ccccc2-c2ccc(N(c3ccccc3-c3ccccc3)c3cccc4c5ccccc5n(-c5ccc(-c6ccccc6)cc5)c34)cc21 ; c1ccc(-c2ccc(N(c3ccc4c5ccccc5n(-c5ccccc5)c4c3)c3cccc4c5ccccc5n(-c5ccc(-c6ccccc6)cc5)c34)cc2)cc1 ; c1ccc(-c2ccc(N(c3ccc4c(c3)C(c3ccccc3)(c3ccccc3)c3ccccc3-4)c3cccc4c5ccccc5n(-c5ccc(-c6ccccc6)cc5)c34)cc2)cc1]

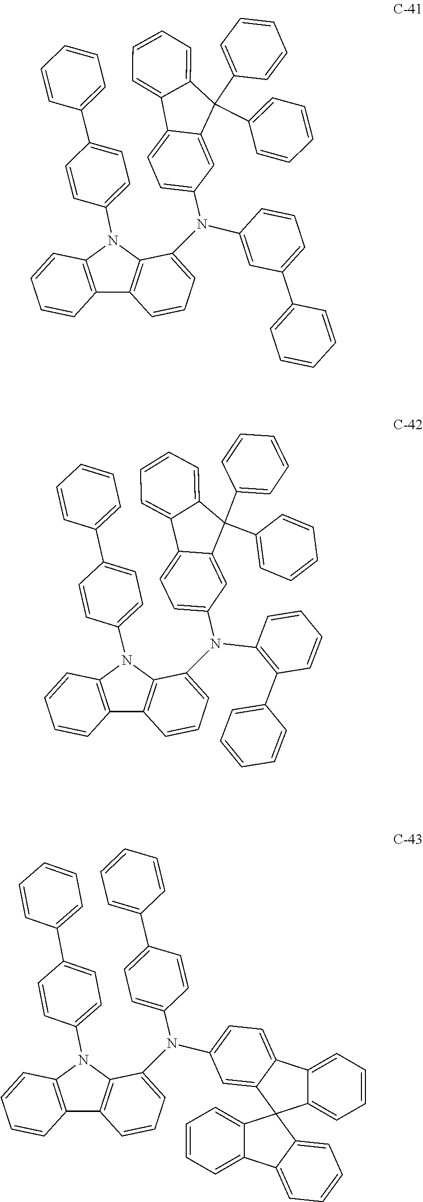 [SMILES: c1ccc(-c2ccc(-n3c4ccccc4c4cccc(N(c5cccc(-c6ccccc6)c5)c5ccc6c(c5)C(c5ccccc5)(c5ccccc5)c5ccccc5-6)c43)cc2)cc1 ; c1ccc(-c2ccc(-n3c4ccccc4c4cccc(N(c5ccc6c(c5)C(c5ccccc5)(c5ccccc5)c5ccccc5-6)c5ccccc5-c5ccccc5)c43)cc2)cc1 ; c1ccc(-c2ccc(N(c3ccc4c(c3)C3(c5ccccc5-c5ccccc53)c3ccccc3-4)c3cccc4c5ccccc5n(-c5ccc(-c6ccccc6)cc5)c34)cc2)cc1]

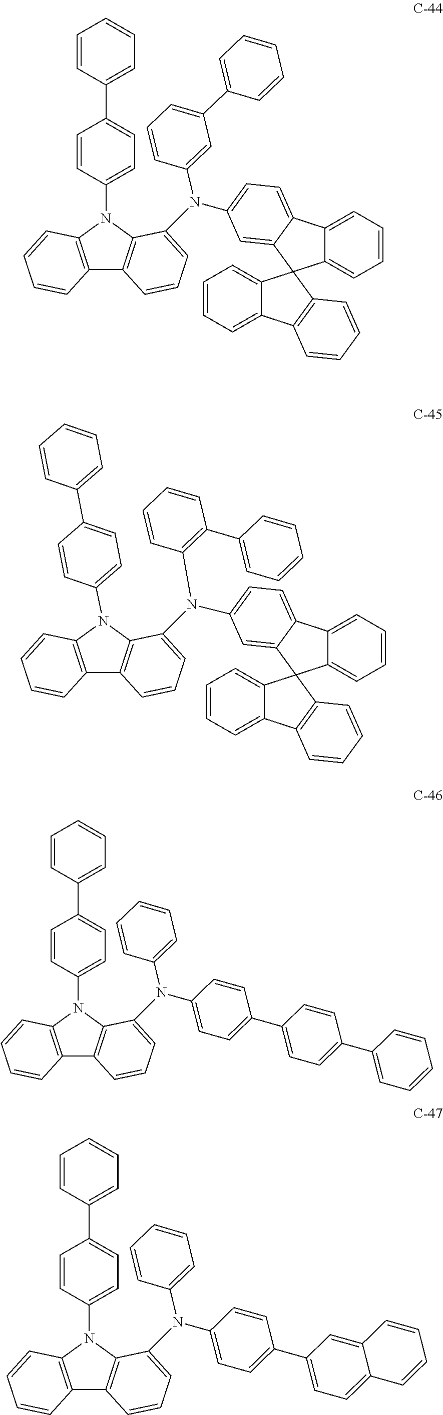 [SMILES: c1ccc(-c2ccc(-n3c4ccccc4c4cccc(N(c5cccc(-c6ccccc6)c5)c5ccc6c(c5)C5(c7ccccc7-c7ccccc75)c5ccccc5-6)c43)cc2)cc1 ; c1ccc(-c2ccc(-n3c4ccccc4c4cccc(N(c5ccc6c(c5)C5(c7ccccc7-c7ccccc75)c5ccccc5-6)c5ccccc5-c5ccccc5)c43)cc2)cc1 ; c1ccc(-c2ccc(-c3ccc(N(c4ccccc4)c4cccc5c6ccccc6n(-c6ccc(-c7ccccc7)cc6)c45)cc3)cc2)cc1 ; c1ccc(-c2ccc(-n3c4ccccc4c4cccc(N(c5ccccc5)c5ccc(-c6ccc7ccccc7c6)cc5)c43)cc2)cc1]

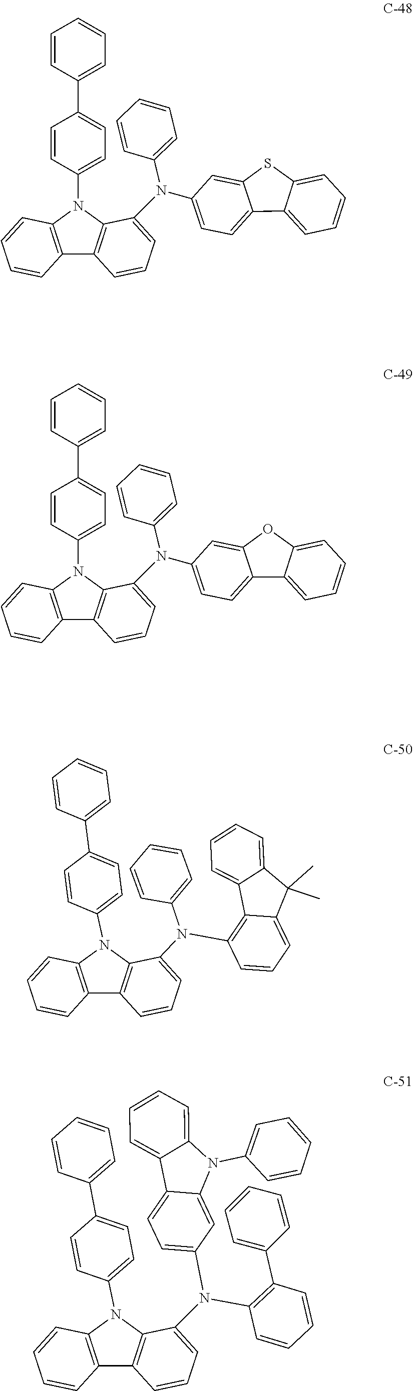 [SMILES: c1ccc(-c2ccc(-n3c4ccccc4c4cccc(N(c5ccccc5)c5ccc6c(c5)sc5ccccc56)c43)cc2)cc1 ; c1ccc(-c2ccc(-n3c4ccccc4c4cccc(N(c5ccccc5)c5ccc6c(c5)oc5ccccc56)c43)cc2)cc1 ; CC1(C)c2ccccc2-c2c(N(c3ccccc3)c3cccc4c5ccccc5n(-c5ccc(-c6ccccc6)cc5)c34)cccc21 ; c1ccc(-c2ccc(-n3c4ccccc4c4cccc(N(c5ccc6c7ccccc7n(-c7ccccc7)c6c5)c5ccccc5-c5ccccc5)c43)cc2)cc1]

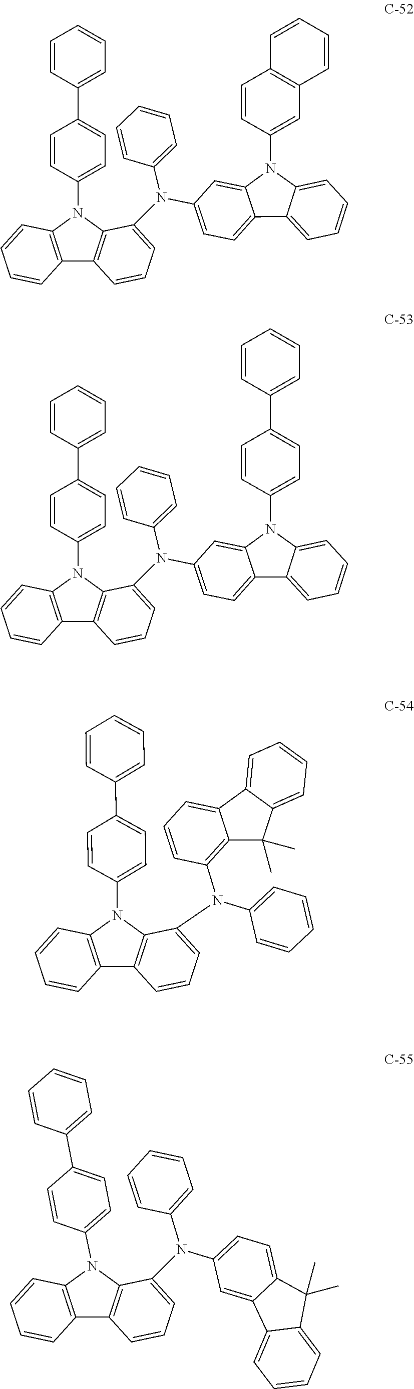 [SMILES: c1ccc(-c2ccc(-n3c4ccccc4c4cccc(N(c5ccccc5)c5ccc6c7ccccc7n(-c7ccc8ccccc8c7)c6c5)c43)cc2)cc1 ; c1ccc(-c2ccc(-n3c4ccccc4c4ccc(N(c5ccccc5)c5cccc6c7ccccc7n(-c7ccc(-c8ccccc8)cc7)c56)cc43)cc2)cc1 ; CC1(C)c2ccccc2-c2cccc(N(c3ccccc3)c3cccc4c5ccccc5n(-c5ccc(-c6ccccc6)cc5)c34)c21 ; CC1(C)c2ccccc2-c2cc(N(c3ccccc3)c3cccc4c5ccccc5n(-c5ccc(-c6ccccc6)cc5)c34)ccc21]

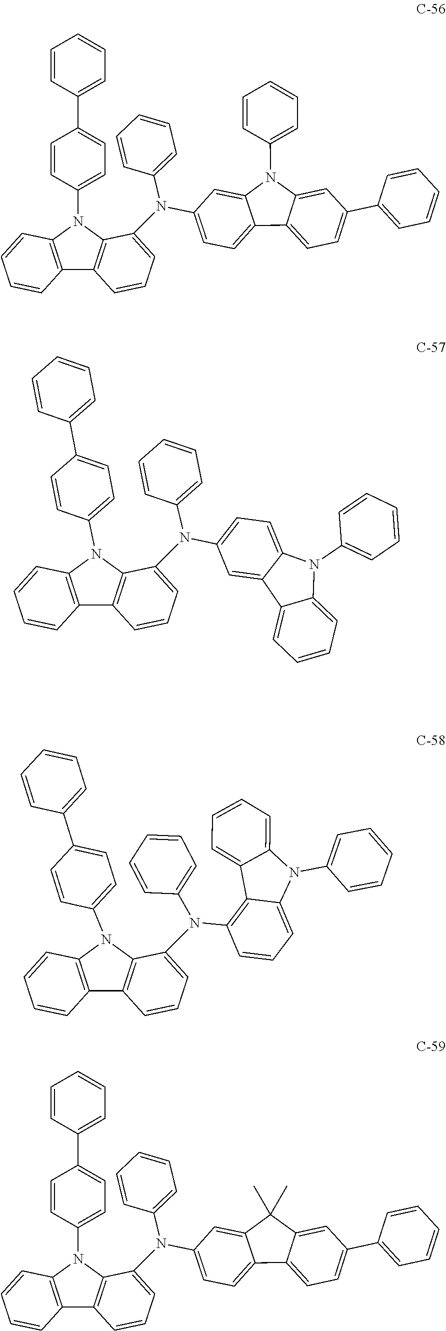 [SMILES: c1ccc(-c2ccc(-n3c4ccccc4c4cccc(N(c5ccccc5)c5ccc6c7ccc(-c8ccccc8)cc7n(-c7ccccc7)c6c5)c43)cc2)cc1 ; c1ccc(-c2ccc(-n3c4ccccc4c4cccc(N(c5ccccc5)c5ccc6c(c5)c5ccccc5n6-c5ccccc5)c43)cc2)cc1 ; c1ccc(-c2ccc(-n3c4ccccc4c4cccc(N(c5ccccc5)c5cccc6c5c5ccccc5n6-c5ccccc5)c43)cc2)cc1 ; CC1(C)c2cc(-c3ccccc3)ccc2-c2ccc(N(c3ccccc3)c3cccc4c5ccccc5n(-c5ccc(-c6ccccc6)cc5)c34)cc21]

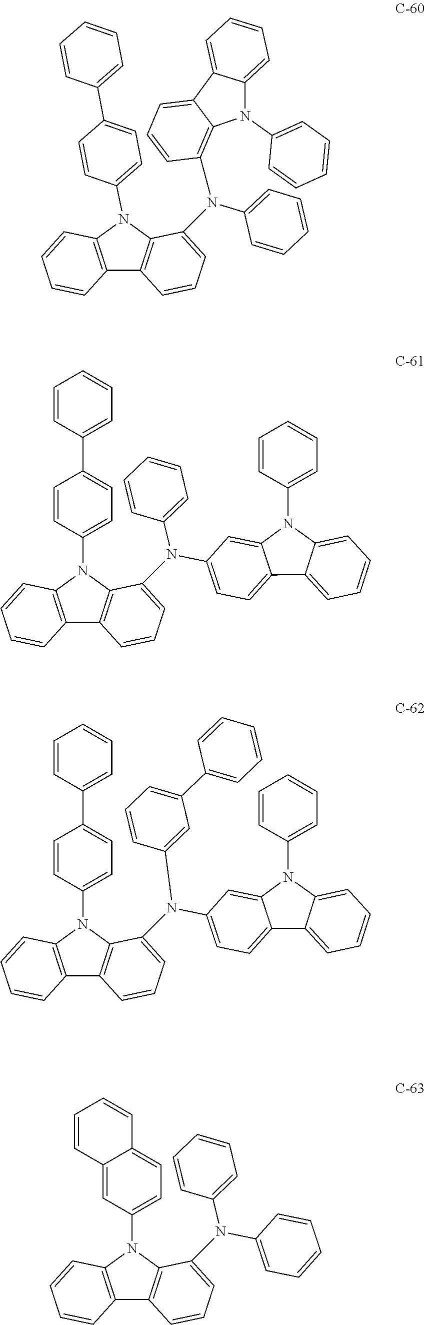 [SMILES: c1ccc(-c2ccc(-n3c4ccccc4c4cccc(N(c5ccccc5)c5cccc6c7ccccc7n(-c7ccccc7)c56)c43)cc2)cc1 ; c1ccc(-c2ccc(-n3c4ccccc4c4cccc(N(c5ccccc5)c5ccc6c7ccccc7n(-c7ccccc7)c6c5)c43)cc2)cc1 ; c1ccc(-c2ccc(-n3c4ccccc4c4cccc(N(c5cccc(-c6ccccc6)c5)c5ccc6c7ccccc7n(-c7ccccc7)c6c5)c43)cc2)cc1 ; c1ccc(N(c2ccccc2)c2cccc3c4ccccc4n(-c4ccc5ccccc5c4)c23)cc1]

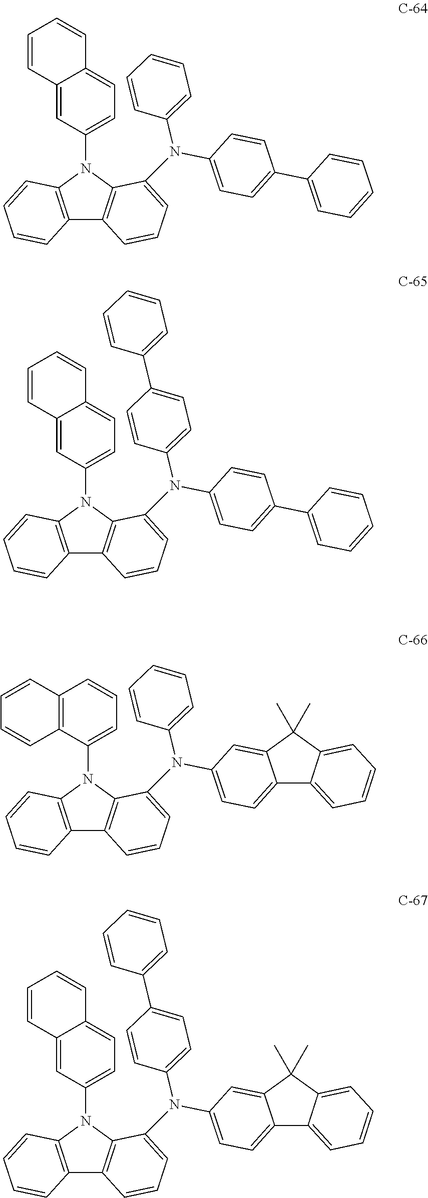 [SMILES: c1ccc(-c2ccc(N(c3ccccc3)c3cccc4c5ccccc5n(-c5ccc6ccccc6c5)c34)cc2)cc1 ; c1ccc(-c2ccc(N(c3ccc(-c4ccccc4)cc3)c3cccc4c5ccccc5n(-c5ccc6ccccc6c5)c34)cc2)cc1 ; CC1(C)c2ccccc2-c2ccc(N(c3ccccc3)c3cccc4c5ccccc5n(-c5cccc6ccccc56)c34)cc21 ; CC1(C)c2ccccc2-c2ccc(N(c3ccc(-c4ccccc4)cc3)c3cccc4c5ccccc5n(-c5ccc6ccccc6c5)c34)cc21]

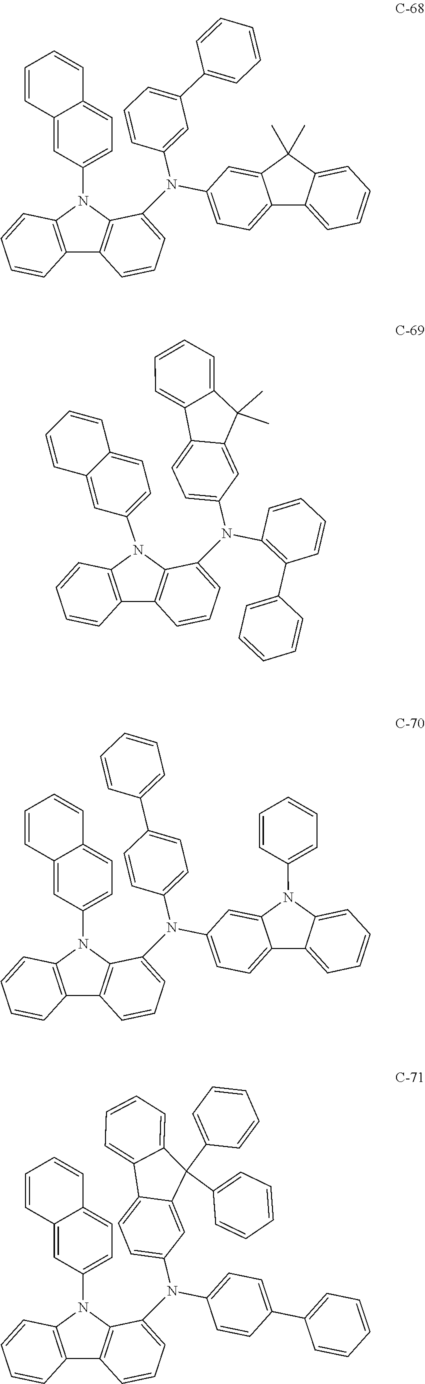 [SMILES: CC1(C)c2ccccc2-c2ccc(N(c3cccc(-c4ccccc4)c3)c3cccc4c5ccccc5n(-c5ccc6ccccc6c5)c34)cc21 ; CC1(C)c2ccccc2-c2ccc(N(c3ccccc3-c3ccccc3)c3cccc4c5ccccc5n(-c5ccc6ccccc6c5)c34)cc21 ; c1ccc(-c2ccc(N(c3ccc4c5ccccc5n(-c5ccccc5)c4c3)c3cccc4c5ccccc5n(-c5ccc6ccccc6c5)c34)cc2)cc1 ; c1ccc(-c2ccc(N(c3ccc4c(c3)C(c3ccccc3)(c3ccccc3)c3ccccc3-4)c3cccc4c5ccccc5n(-c5ccc6ccccc6c5)c34)cc2)cc1]

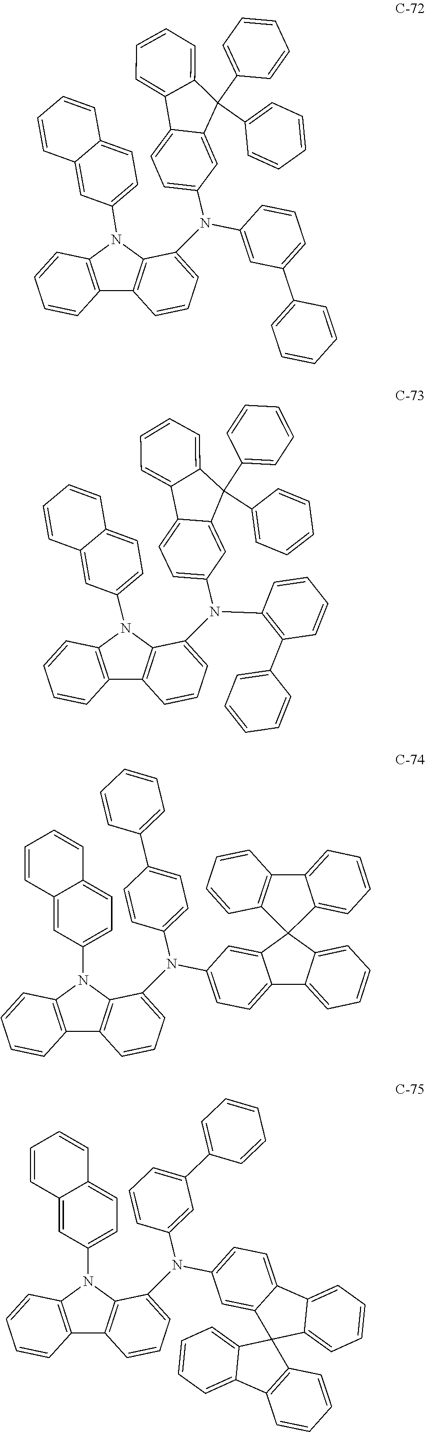 [SMILES: c1ccc(-c2cccc(N(c3ccc4c(c3)C(c3ccccc3)(c3ccccc3)c3ccccc3-4)c3cccc4c5ccccc5n(-c5ccc6ccccc6c5)c34)c2)cc1 ; c1ccc(-c2ccccc2N(c2ccc3c(c2)C(c2ccccc2)(c2ccccc2)c2ccccc2-3)c2cccc3c4ccccc4n(-c4ccc5ccccc5c4)c23)cc1 ; c1ccc(-c2ccc(N(c3ccc4c(c3)C3(c5ccccc5-c5ccccc53)c3ccccc3-4)c3cccc4c5ccccc5n(-c5ccc6ccccc6c5)c34)cc2)cc1 ; c1ccc(-c2cccc(N(c3ccc4c(c3)C3(c5ccccc5-c5ccccc53)c3ccccc3-4)c3cccc4c5ccccc5n(-c5ccc6ccccc6c5)c34)c2)cc1]

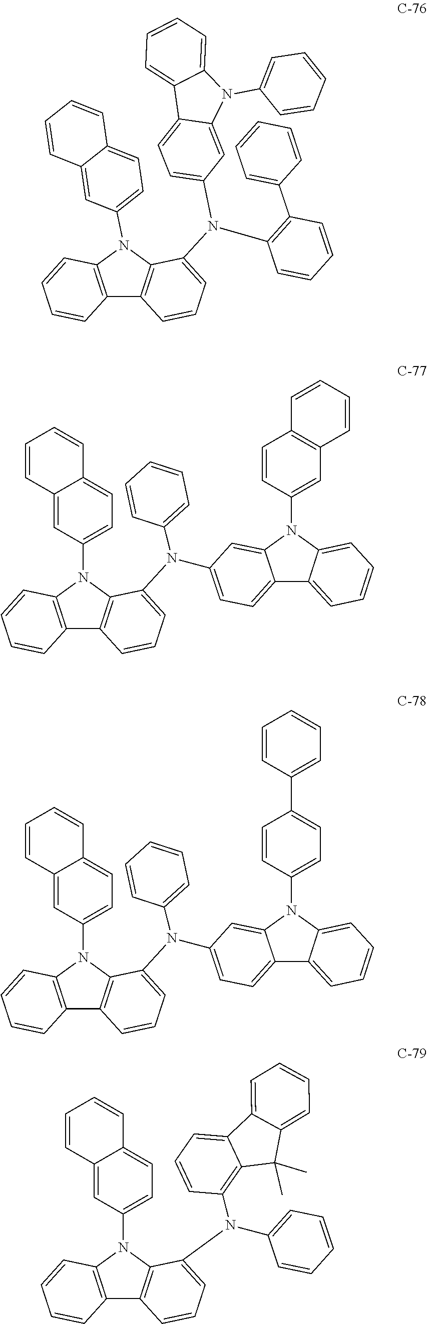 [SMILES: c1ccc(-c2ccccc2N(c2ccc3c4ccccc4n(-c4ccccc4)c3c2)c2cccc3c4ccccc4n(-c4ccc5ccccc5c4)c23)cc1 ; c1ccc(N(c2ccc3c4ccccc4n(-c4ccc5ccccc5c4)c3c2)c2cccc3c4ccccc4n(-c4ccc5ccccc5c4)c23)cc1 ; c1ccc(-c2ccc(-n3c4ccccc4c4ccc(N(c5ccccc5)c5cccc6c7ccccc7n(-c7ccc8ccccc8c7)c56)cc43)cc2)cc1 ; CC1(C)c2ccccc2-c2cccc(N(c3ccccc3)c3cccc4c5ccccc5n(-c5ccc6ccccc6c5)c34)c21]

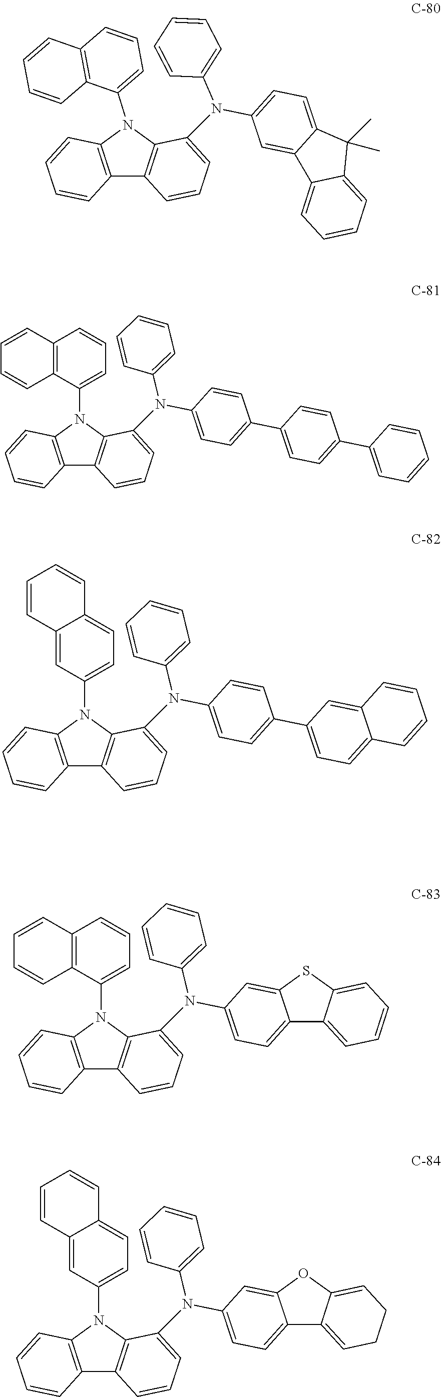 [SMILES: CC1(C)c2ccccc2-c2cc(N(c3ccccc3)c3cccc4c5ccccc5n(-c5cccc6ccccc56)c34)ccc21 ; c1ccc(-c2ccc(-c3ccc(N(c4ccccc4)c4cccc5c6ccccc6n(-c6cccc7ccccc67)c45)cc3)cc2)cc1 ; c1ccc(N(c2ccc(-c3ccc4ccccc4c3)cc2)c2cccc3c4ccccc4n(-c4ccc5ccccc5c4)c23)cc1 ; c1ccc(N(c2ccc3c(c2)sc2ccccc23)c2cccc3c4ccccc4n(-c4cccc5ccccc45)c23)cc1 ; C1=c2oc3cc(N(c4ccccc4)c4cccc5c6ccccc6n(-c6ccc7ccccc7c6)c45)ccc3c2=CCC1]

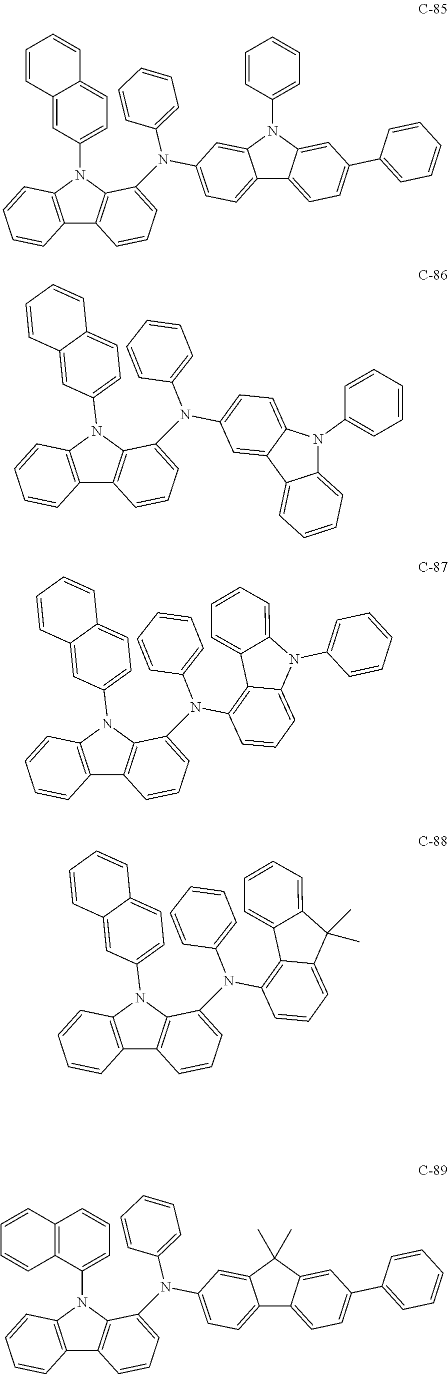

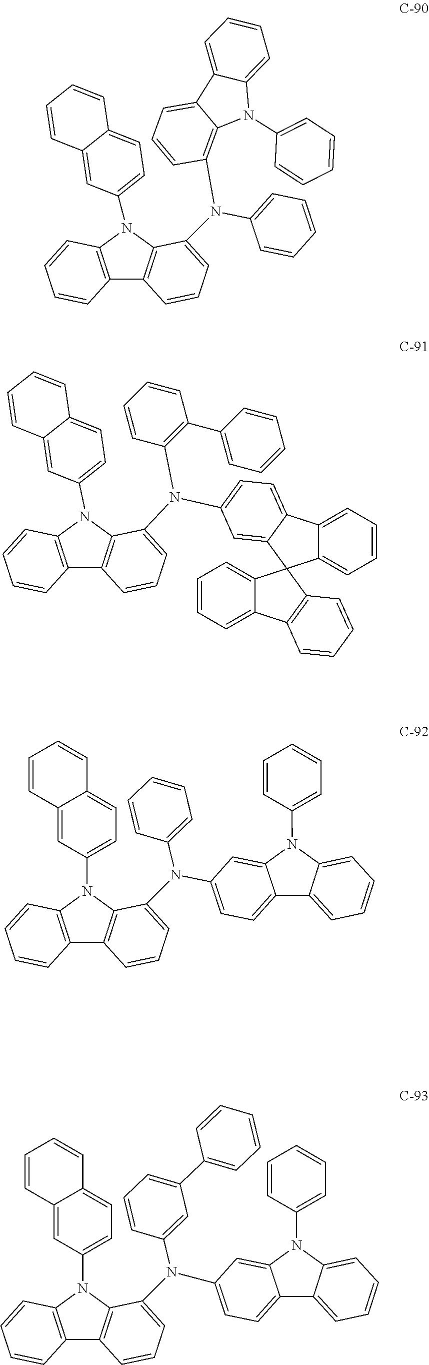 [SMILES: c1ccc(N(c2cccc3c4ccccc4n(-c4ccccc4)c23)c2cccc3c4ccccc4n(-c4ccc5ccccc5c4)c23)cc1 ; c1ccc(-c2ccccc2N(c2ccc3c(c2)C2(c4ccccc4-c4ccccc42)c2ccccc2-3)c2cccc3c4ccccc4n(-c4ccc5ccccc5c4)c23)cc1 ; c1ccc(N(c2ccc3c4ccccc4n(-c4ccccc4)c3c2)c2cccc3c4ccccc4n(-c4ccc5ccccc5c4)c23)cc1 ; c1ccc(-c2cccc(N(c3ccc4c5ccccc5n(-c5ccccc5)c4c3)c3cccc4c5ccccc5n(-c5ccc6ccccc6c5)c34)c2)cc1]

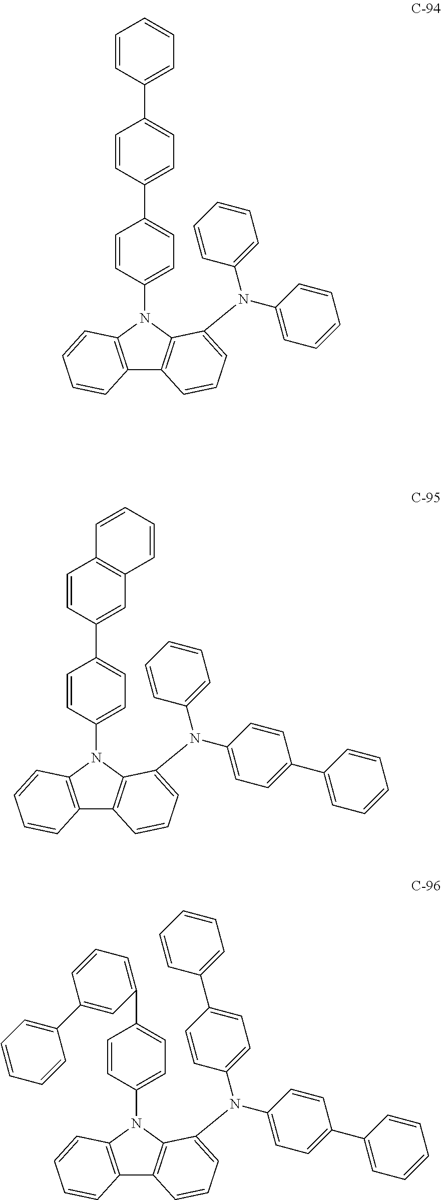 [SMILES: c1ccc(-c2ccc(-c3ccc(-n4c5ccccc5c5cccc(N(c6ccccc6)c6ccccc6)c54)cc3)cc2)cc1 ; c1ccc(-c2ccc(N(c3ccccc3)c3cccc4c5ccccc5n(-c5ccc(-c6ccc7ccccc7c6)cc5)c34)cc2)cc1 ; c1ccc(-c2ccc(N(c3ccc(-c4ccccc4)cc3)c3cccc4c5ccccc5n(-c5ccc(-c6cccc(-c7ccccc7)c6)cc5)c34)cc2)cc1]

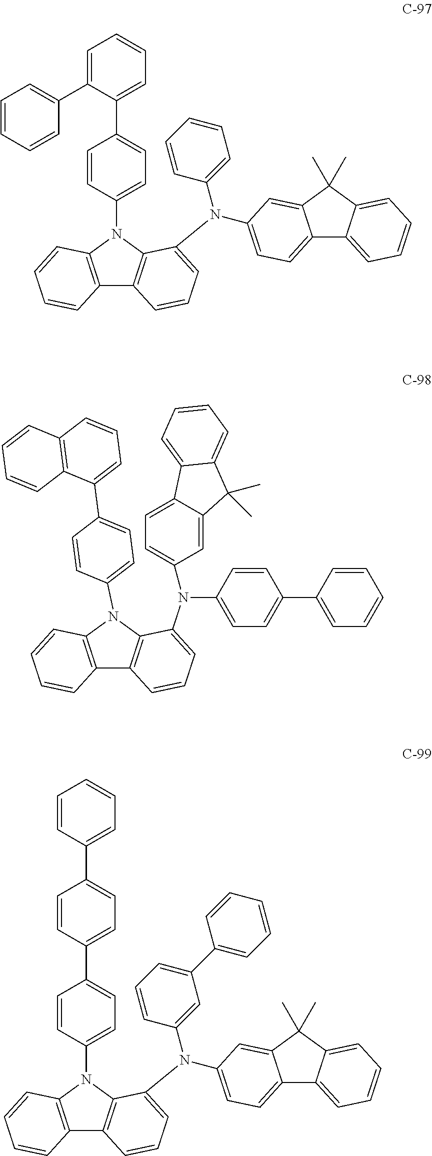 [SMILES: CC1(C)c2ccccc2-c2ccc(N(c3ccccc3)c3cccc4c5ccccc5n(-c5ccc(-c6ccccc6-c6ccccc6)cc5)c34)cc21 ; CC1(C)c2ccccc2-c2ccc(N(c3ccc(-c4ccccc4)cc3)c3cccc4c5ccccc5n(-c5ccc(-c6cccc7ccccc67)cc5)c34)cc21 ; CC1(C)c2ccccc2-c2ccc(N(c3cccc(-c4ccccc4)c3)c3cccc4c5ccccc5n(-c5ccc(-c6ccc(-c7ccccc7)cc6)cc5)c34)cc21]

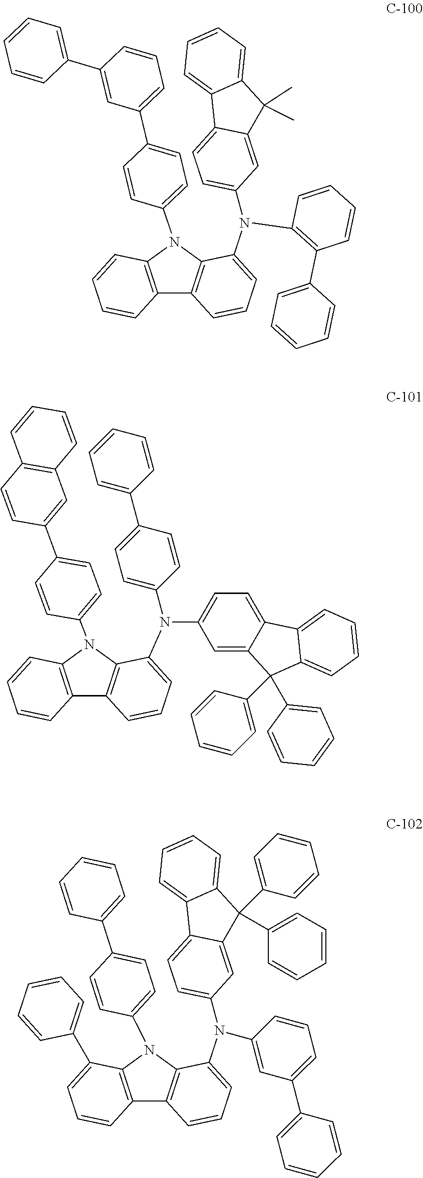 [SMILES: CC1(C)c2ccccc2-c2ccc(N(c3ccccc3-c3ccccc3)c3cccc4c5ccccc5n(-c5ccc(-c6cccc(-c7ccccc7)c6)cc5)c34)cc21 ; c1ccc(-c2ccc(N(c3ccc4c(c3)C(c3ccccc3)(c3ccccc3)c3ccccc3-4)c3cccc4c5ccccc5n(-c5ccc(-c6ccc7ccccc7c6)cc5)c34)cc2)cc1 ; c1ccc(-c2ccc(-n3c4c(-c5ccccc5)cccc4c4cccc(N(c5cccc(-c6ccccc6)c5)c5ccc6c(c5)C(c5ccccc5)(c5ccccc5)c5ccccc5-6)c43)cc2)cc1]

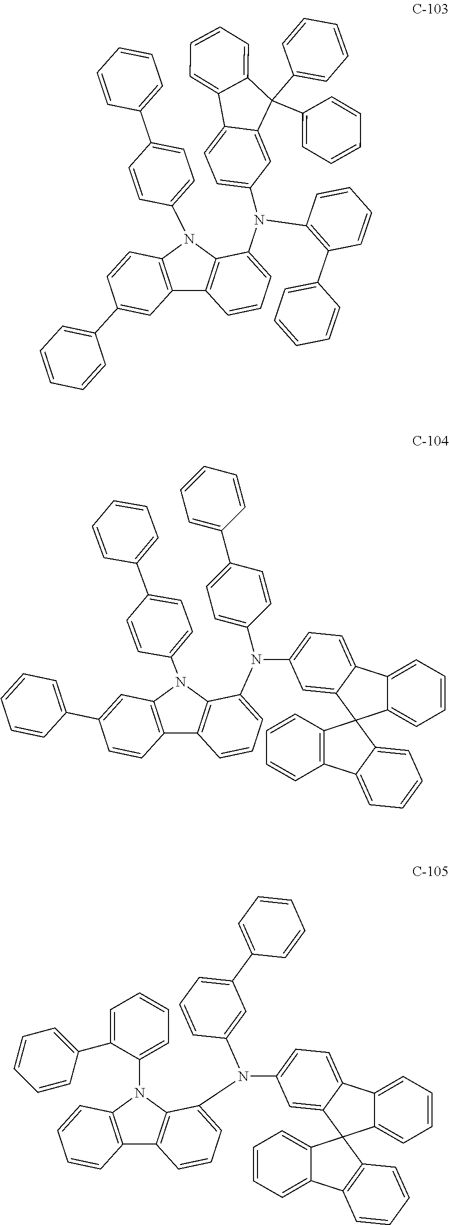 [SMILES: c1ccc(-c2ccc(-n3c4ccc(-c5ccccc5)cc4c4cccc(N(c5ccc6c(c5)C(c5ccccc5)(c5ccccc5)c5ccccc5-6)c5ccccc5-c5ccccc5)c43)cc2)cc1 ; c1ccc(-c2ccc(N(c3ccc4c(c3)C3(c5ccccc5-c5ccccc53)c3ccccc3-4)c3cccc4c5ccc(-c6ccccc6)cc5n(-c5ccc(-c6ccccc6)cc5)c34)cc2)cc1 ; c1ccc(-c2cccc(N(c3ccc4c(c3)C3(c5ccccc5-c5ccccc53)c3ccccc3-4)c3cccc4c5ccccc5n(-c5ccccc5-c5ccccc5)c34)c2)cc1]

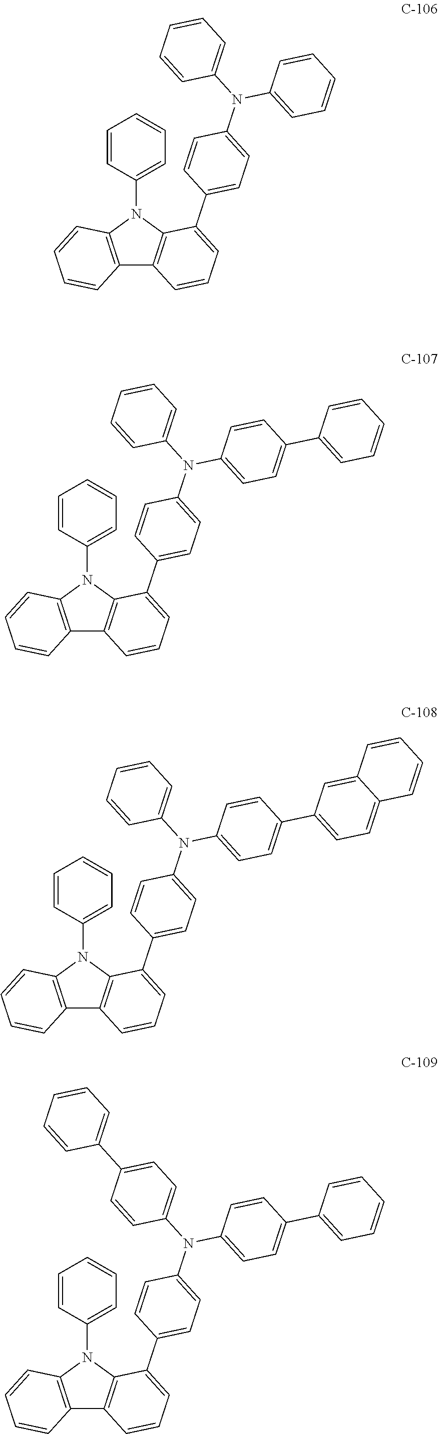 [SMILES: c1ccc(N(c2ccccc2)c2ccc(-c3cccc4c5ccccc5n(-c5ccccc5)c34)cc2)cc1 ; c1ccc(-c2ccc(N(c3ccccc3)c3ccc(-c4cccc5c6ccccc6n(-c6ccccc6)c45)cc3)cc2)cc1 ; c1ccc(N(c2ccc(-c3ccc4ccccc4c3)cc2)c2ccc(-c3cccc4c5ccccc5n(-c5ccccc5)c34)cc2)cc1 ; c1ccc(-c2ccc(N(c3ccc(-c4ccccc4)cc3)c3ccc(-c4cccc5c6ccccc6n(-c6ccccc6)c45)cc3)cc2)cc1]

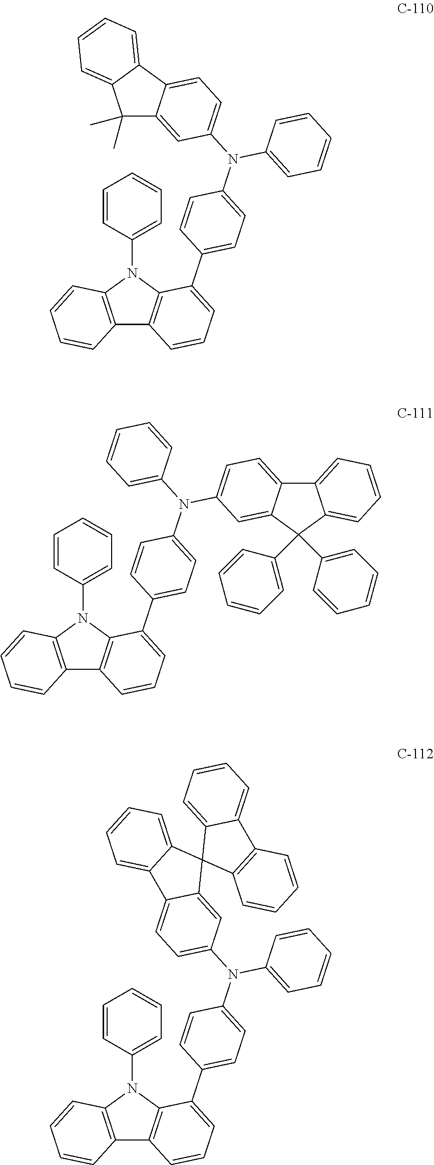 [SMILES: CC1(C)c2ccccc2-c2ccc(N(c3ccccc3)c3ccc(-c4cccc5c6ccccc6n(-c6ccccc6)c45)cc3)cc21 ; c1ccc(N(c2ccc(-c3cccc4c5ccccc5n(-c5ccccc5)c34)cc2)c2ccc3c(c2)C(c2ccccc2)(c2ccccc2)c2ccccc2-3)cc1 ; c1ccc(N(c2ccc(-c3cccc4c5ccccc5n(-c5ccccc5)c34)cc2)c2ccc3c(c2)C2(c4ccccc4-c4ccccc42)c2ccccc2-3)cc1]

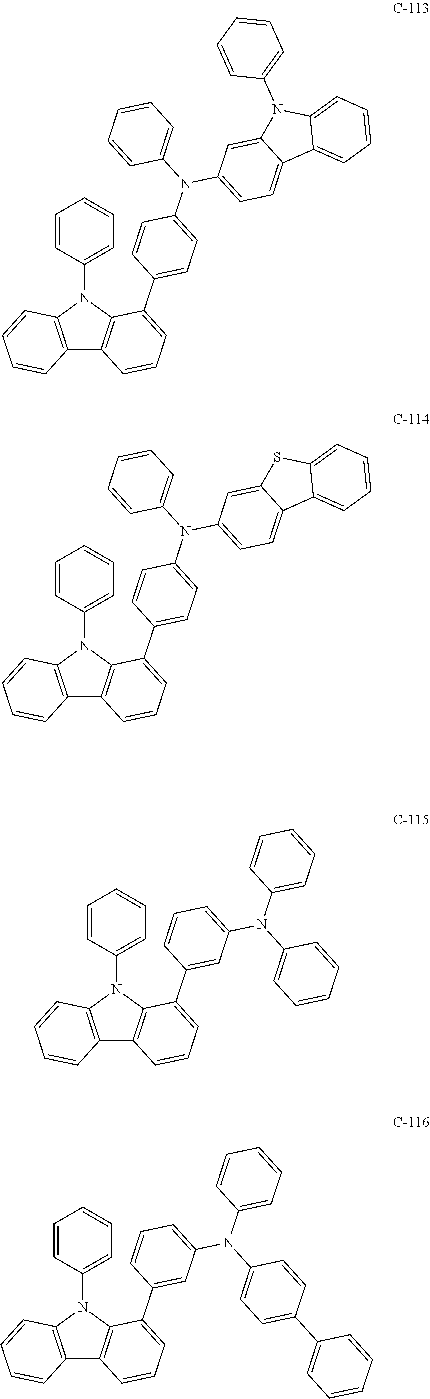 [SMILES: c1ccc(N(c2ccc(-c3cccc4c5ccccc5n(-c5ccccc5)c34)cc2)c2ccc3c4ccccc4n(-c4ccccc4)c3c2)cc1 ; c1ccc(N(c2ccc(-c3cccc4c5ccccc5n(-c5ccccc5)c34)cc2)c2ccc3c(c2)sc2ccccc23)cc1 ; c1ccc(N(c2ccccc2)c2cccc(-c3cccc4c5ccccc5n(-c5ccccc5)c34)c2)cc1 ; c1ccc(-c2ccc(N(c3ccccc3)c3cccc(-c4cccc5c6ccccc6n(-c6ccccc6)c45)c3)cc2)cc1]

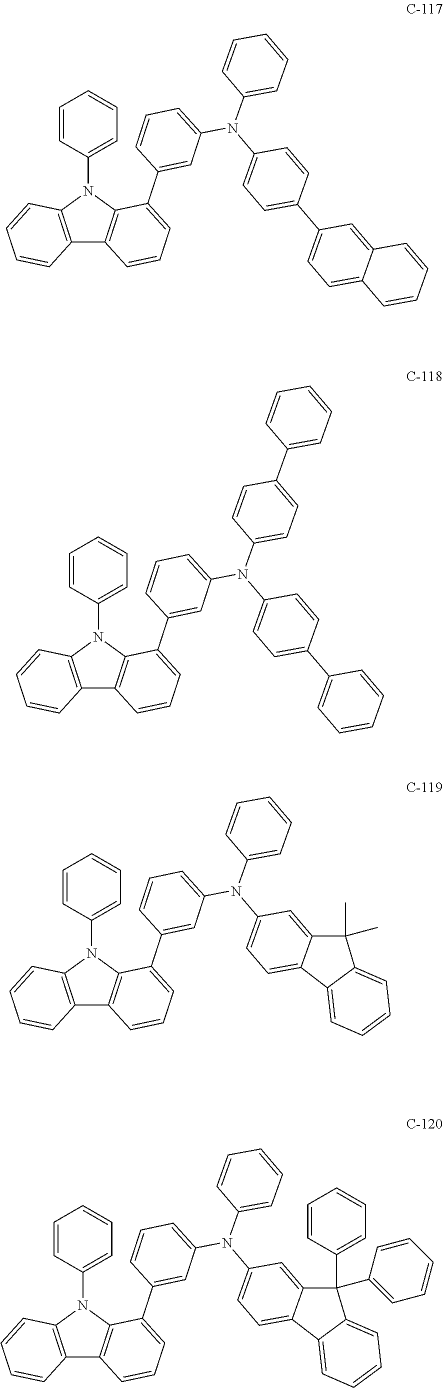 [SMILES: c1ccc(N(c2ccc(-c3ccc4ccccc4c3)cc2)c2cccc(-c3cccc4c5ccccc5n(-c5ccccc5)c34)c2)cc1 ; c1ccc(-c2ccc(N(c3ccc(-c4ccccc4)cc3)c3cccc(-c4cccc5c6ccccc6n(-c6ccccc6)c45)c3)cc2)cc1 ; CC1(C)c2ccccc2-c2ccc(N(c3ccccc3)c3cccc(-c4cccc5c6ccccc6n(-c6ccccc6)c45)c3)cc21 ; c1ccc(N(c2cccc(-c3cccc4c5ccccc5n(-c5ccccc5)c34)c2)c2ccc3c(c2)C(c2ccccc2)(c2ccccc2)c2ccccc2-3)cc1]

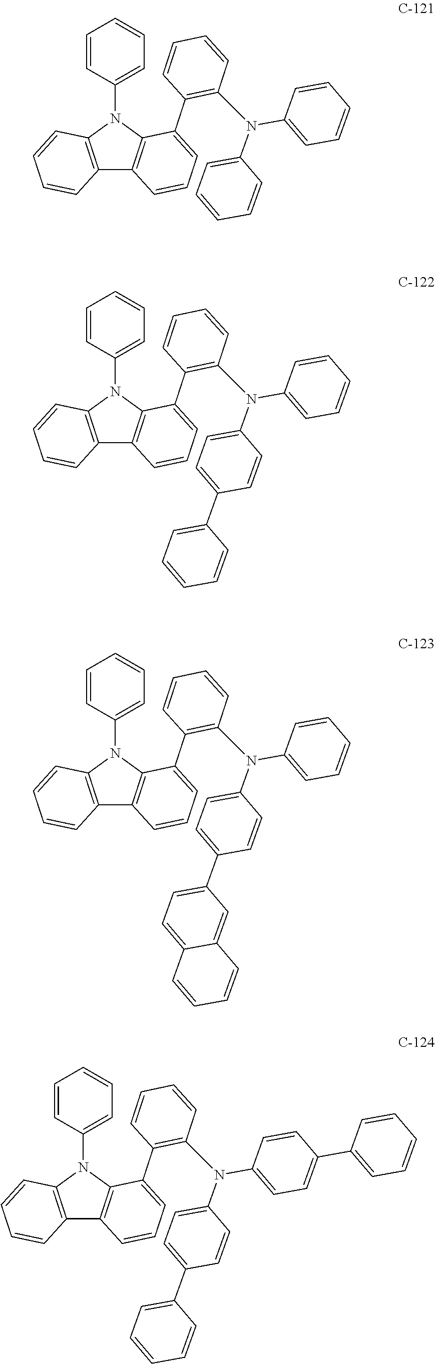 [SMILES: c1ccc(N(c2ccccc2)c2ccccc2-c2cccc3c4ccccc4n(-c4ccccc4)c23)cc1 ; c1ccc(-c2ccc(N(c3ccccc3)c3ccccc3-c3cccc4c5ccccc5n(-c5ccccc5)c34)cc2)cc1 ; c1ccc(N(c2ccc(-c3ccc4ccccc4c3)cc2)c2ccccc2-c2cccc3c4ccccc4n(-c4ccccc4)c23)cc1 ; c1ccc(-c2ccc(N(c3ccc(-c4ccccc4)cc3)c3ccccc3-c3cccc4c5ccccc5n(-c5ccccc5)c34)cc2)cc1]

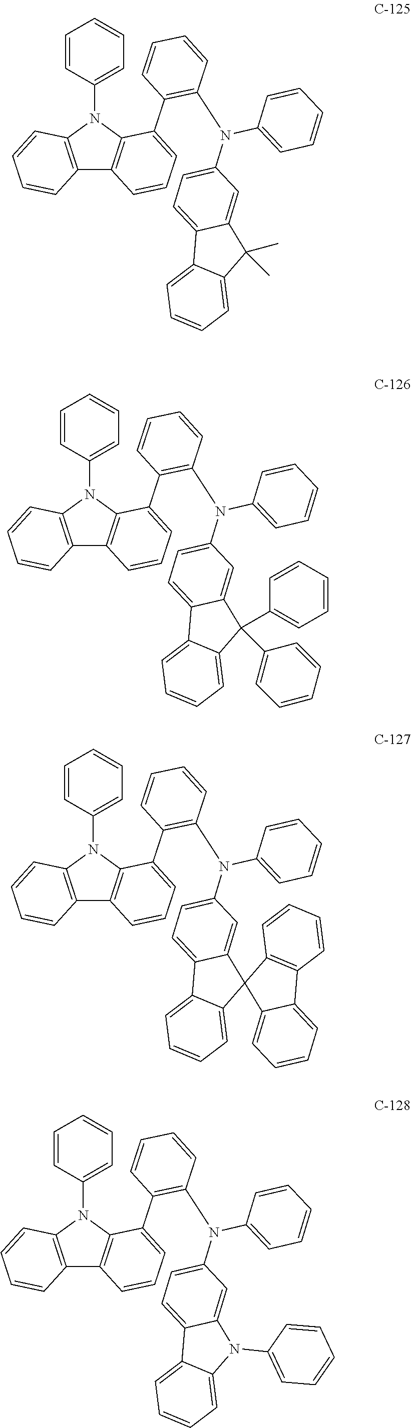 [SMILES: CC1(C)c2ccccc2-c2ccc(N(c3ccccc3)c3ccccc3-c3cccc4c5ccccc5n(-c5ccccc5)c34)cc21 ; c1ccc(N(c2ccc3c(c2)C(c2ccccc2)(c2ccccc2)c2ccccc2-3)c2ccccc2-c2cccc3c4ccccc4n(-c4ccccc4)c23)cc1 ; c1ccc(N(c2ccc3c(c2)C2(c4ccccc4-c4ccccc42)c2ccccc2-3)c2ccccc2-c2cccc3c4ccccc4n(-c4ccccc4)c23)cc1 ; c1ccc(N(c2ccc3c4ccccc4n(-c4ccccc4)c3c2)c2ccccc2-c2cccc3c4ccccc4n(-c4ccccc4)c23)cc1]

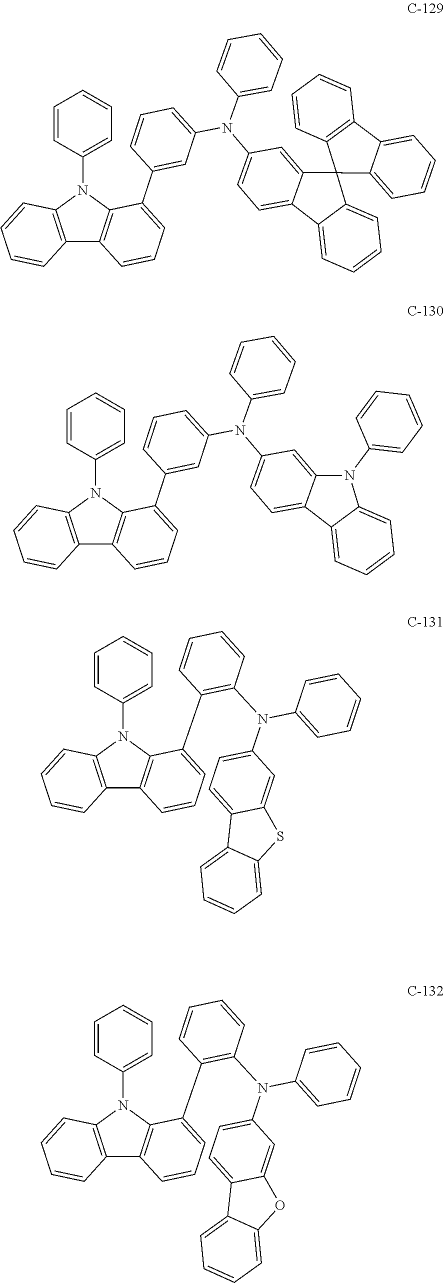 [SMILES: c1ccc(N(c2cccc(-c3cccc4c5ccccc5n(-c5ccccc5)c34)c2)c2ccc3c(c2)C2(c4ccccc4-c4ccccc42)c2ccccc2-3)cc1 ; c1ccc(N(c2cccc(-c3cccc4c5ccccc5n(-c5ccccc5)c34)c2)c2ccc3c4ccccc4n(-c4ccccc4)c3c2)cc1 ; c1ccc(N(c2ccc3c(c2)sc2ccccc23)c2ccccc2-c2cccc3c4ccccc4n(-c4ccccc4)c23)cc1 ; c1ccc(N(c2ccc3c(c2)oc2ccccc23)c2ccccc2-c2cccc3c4ccccc4n(-c4ccccc4)c23)cc1]

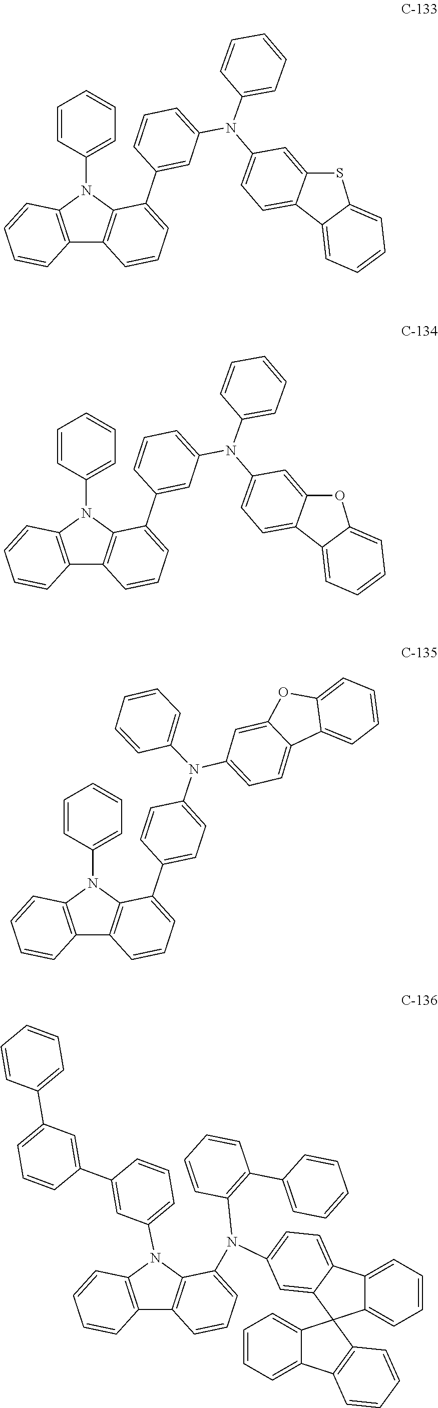 [SMILES: c1ccc(N(c2cccc(-c3cccc4c5ccccc5n(-c5ccccc5)c34)c2)c2ccc3c(c2)sc2ccccc23)cc1 ; c1ccc(N(c2cccc(-c3cccc4c5ccccc5n(-c5ccccc5)c34)c2)c2ccc3c(c2)oc2ccccc23)cc1 ; c1ccc(N(c2ccc(-c3cccc4c5ccccc5n(-c5ccccc5)c34)cc2)c2ccc3c(c2)oc2ccccc23)cc1 ; c1ccc(-c2cccc(-c3cccc(-n4c5ccccc5c5cccc(N(c6ccc7c(c6)C6(c8ccccc8-c8ccccc86)c6ccccc6-7)c6ccccc6-c6ccccc6)c54)c3)c2)cc1]

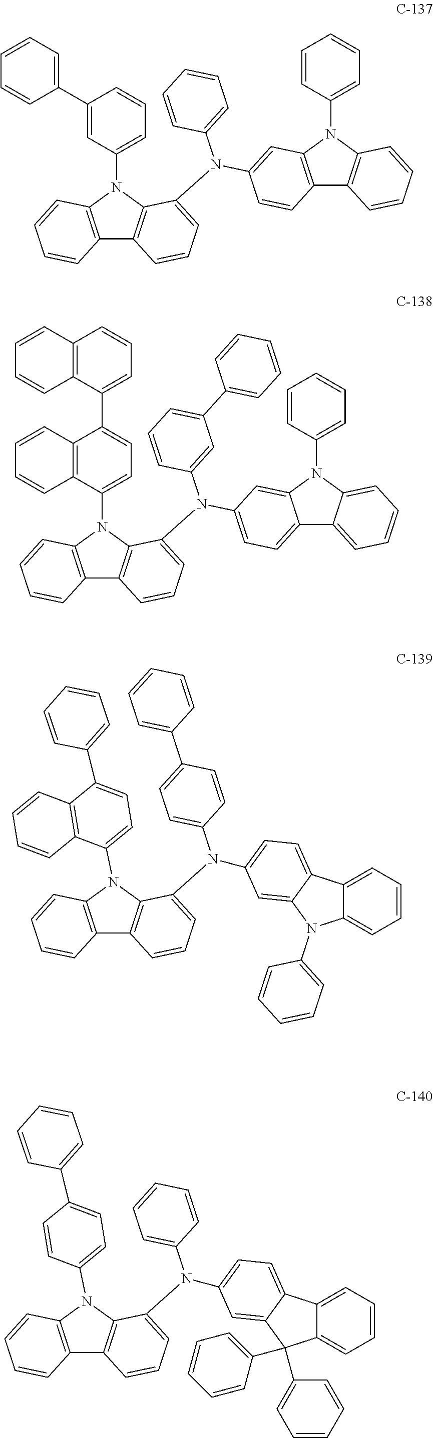 [SMILES: c1ccc(-c2cccc(-n3c4ccccc4c4cccc(N(c5ccccc5)c5ccc6c7ccccc7n(-c7ccccc7)c6c5)c43)c2)cc1 ; c1ccc(-c2cccc(N(c3ccc4c5ccccc5n(-c5ccccc5)c4c3)c3cccc4c5ccccc5n(-c5ccc(-c6cccc7ccccc67)c6ccccc56)c34)c2)cc1 ; c1ccc(-c2ccc(N(c3ccc4c5ccccc5n(-c5ccccc5)c4c3)c3cccc4c5ccccc5n(-c5ccc(-c6ccccc6)c6ccccc56)c34)cc2)cc1 ; c1ccc(-c2ccc(-n3c4ccccc4c4cccc(N(c5ccccc5)c5ccc6c(c5)C(c5ccccc5)(c5ccccc5)c5ccccc5-6)c43)cc2)cc1]

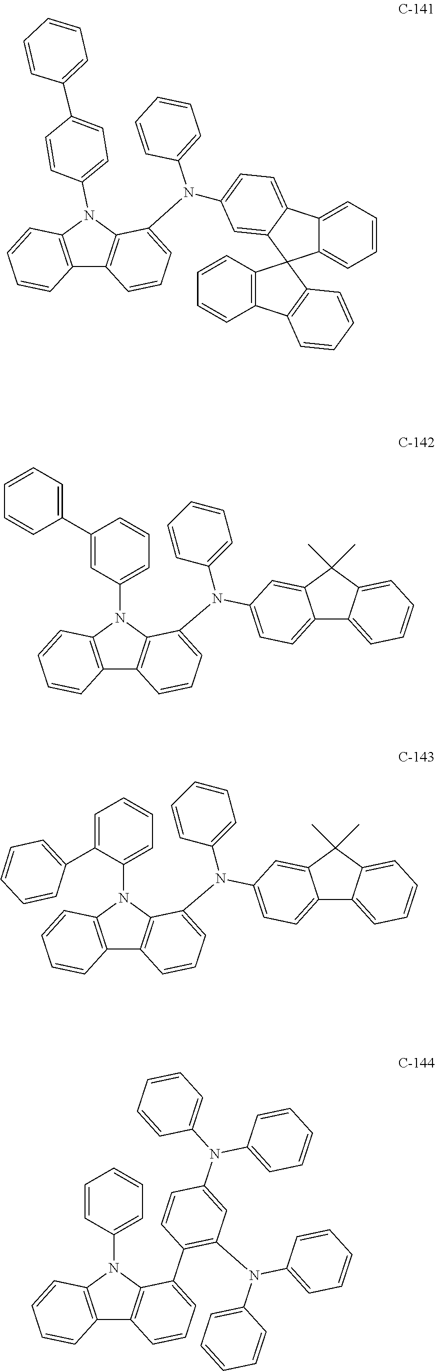 [SMILES: c1ccc(-c2ccc(-n3c4ccccc4c4cccc(N(c5ccccc5)c5ccc6c(c5)C5(c7ccccc7-c7ccccc75)c5ccccc5-6)c43)cc2)cc1 ; CC1(C)c2ccccc2-c2ccc(N(c3ccccc3)c3cccc4c5ccccc5n(-c5cccc(-c6ccccc6)c5)c34)cc21 ; CC1(C)c2ccccc2-c2ccc(N(c3ccccc3)c3cccc4c5ccccc5n(-c5ccccc5-c5ccccc5)c34)cc21 ; c1ccc(N(c2ccccc2)c2ccc(-c3cccc4c5ccccc5n(-c5ccccc5)c34)c(N(c3ccccc3)c3ccccc3)c2)cc1]

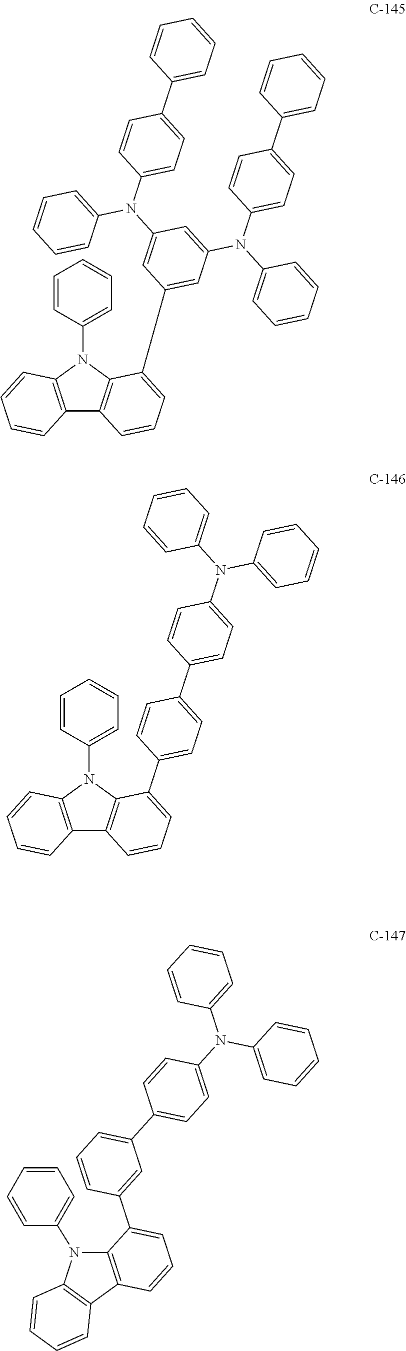 [SMILES: c1ccc(-c2ccc(N(c3ccccc3)c3cc(-c4cccc5c6ccccc6n(-c6ccccc6)c45)cc(N(c4ccccc4)c4ccc(-c5ccccc5)cc4)c3)cc2)cc1 ; c1ccc(N(c2ccccc2)c2ccc(-c3ccc(-c4cccc5c6ccccc6n(-c6ccccc6)c45)cc3)cc2)cc1 ; c1ccc(N(c2ccccc2)c2ccc(-c3cccc(-c4cccc5c6ccccc6n(-c6ccccc6)c45)c3)cc2)cc1]

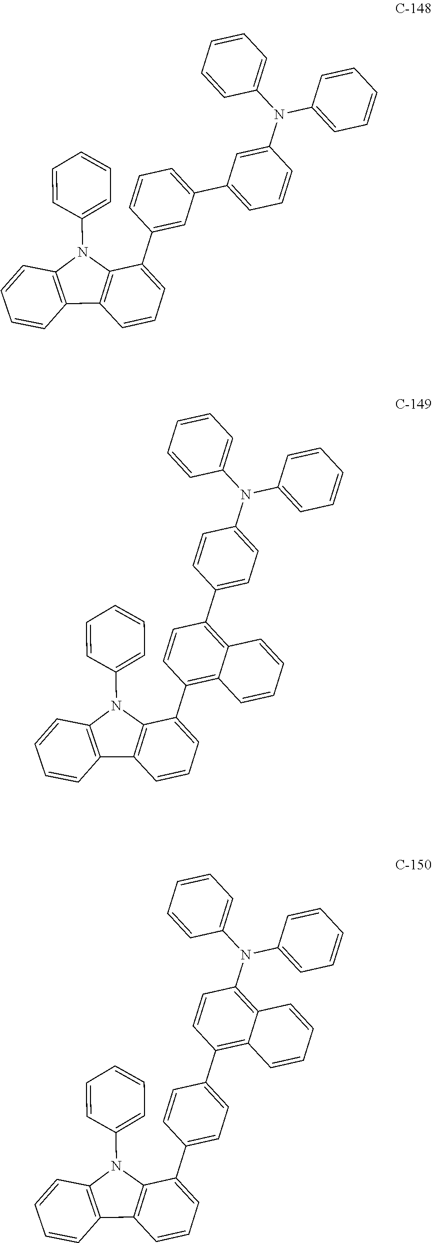 [SMILES: c1ccc(N(c2ccccc2)c2cccc(-c3cccc(-c4cccc5c6ccccc6n(-c6ccccc6)c45)c3)c2)cc1 ; c1ccc(N(c2ccccc2)c2ccc(-c3ccc(-c4cccc5c6ccccc6n(-c6ccccc6)c45)c4ccccc34)cc2)cc1 ; c1ccc(N(c2ccccc2)c2ccc(-c3ccc(-c4cccc5c6ccccc6n(-c6ccccc6)c45)cc3)c3ccccc23)cc1]

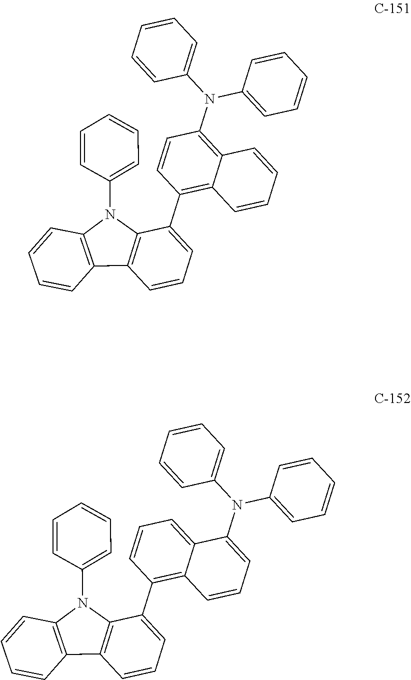 [SMILES: c1ccc(N(c2ccccc2)c2ccc(-c3cccc4c5ccccc5n(-c5ccccc5)c34)c3ccccc23)cc1 ; c1ccc(N(c2ccccc2)c2cccc3c(-c4cccc5c6ccccc6n(-c6ccccc6)c45)cccc23)cc1]

The compound of formula 1 according to the present disclosure may be produced as represented by the following reaction scheme 1 or 2, but is not limited thereto and by a synthetic method known to a person skilled in the art.

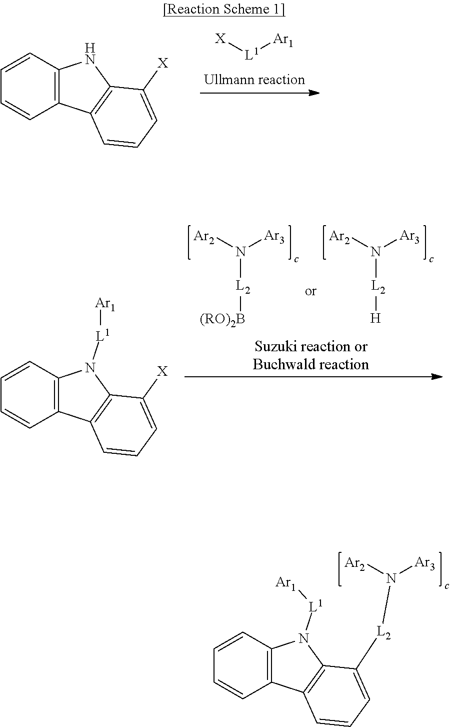

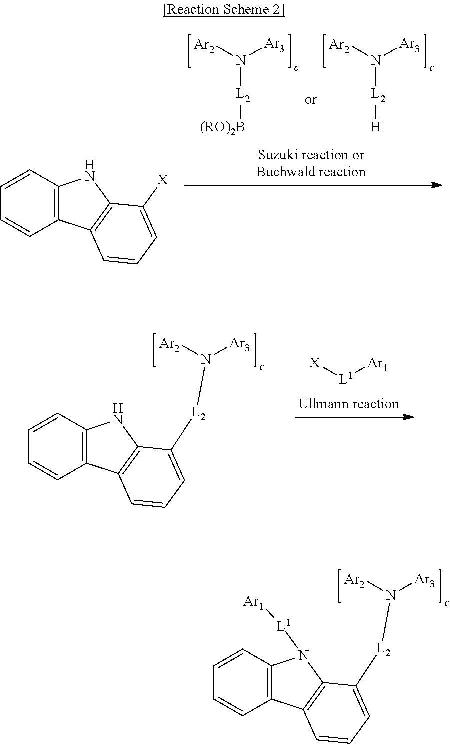

In reaction schemes 1 and 2, the definitions of the substituents are as defined in formula 1.

As described above, exemplary synthesis examples of the compounds represented by formula 1 according to one embodiment are described, but they are based on Buchwald-Hartwig cross coupling reaction, N-arylation reaction, Miyaura borylation reaction, Suzuki cross-coupling reaction, Grignard reaction, Heck reaction, SN₁ substitution reaction, SN₂ substitution reaction, and Phosphine-mediated reductive cyclization reaction etc. It will be understood by one skilled in the art that the above reaction proceeds even if other substituents defined in the formula 1 other than the substituents described in the specific synthesis examples are bonded.

The present disclosure may provide an organic electroluminescent material comprising the organic electroluminescent compound of the formula 1, and an organic electroluminescent device comprising the organic electroluminescent material.

The organic electroluminescent material may be made solely of the organic electroluminescent compound of the present disclosure, or may further comprise conventional materials included in the organic electroluminescent material. When two or more species of materials are included in one layer, the at least two compounds may be a mixture-evaporation or a co-evaporation to form a layer. The organic electroluminescent material according to one embodiment may comprise at least one compound represented by the formula 1. For example, the compound of the formula 1 may be contained in a hole transport layer and/or a light-emitting layer, preferably, the organic electroluminescent compound of the formula 1 of the present disclosure may be contained as a hole transport material of the organic electroluminescent device.

An organic electroluminescent material of the present disclosure may comprise host compound other than the organic electroluminescent compound of the formula 1, preferably, the organic electroluminescent material may further comprise at least one dopant.

The dopant comprised in the organic electroluminescent material of the present disclosure may be at least one phosphorescent or fluorescent dopant, preferably a phosphorescent dopant. The phosphorescent dopant material applied to the organic electroluminescent device of the present disclosure is not particulary limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant may use the compound represented by the following formula 101, but is not limited thereto:

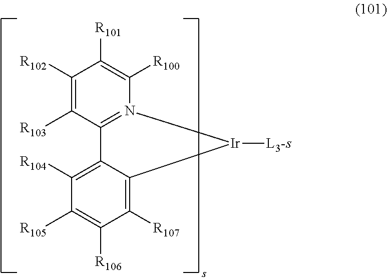

In formula 101,
L is selected from the following structure 1 or 2;

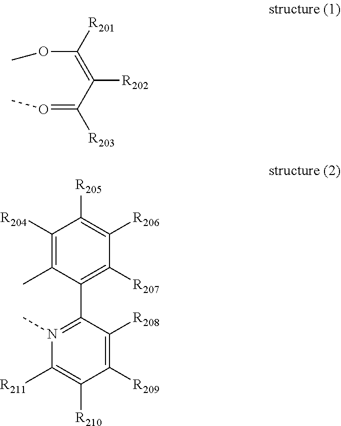

R₁₀₀ to R₁₀₃ each independently represent hydrogen, deuterium, halogen, halogen-substituted or unsubstituted (C1-C30)alkyl, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or R₁₀₀ to R₁₀₃ may be linked to an adjacent substituent(s) to form a substituted or unsubstituted fused ring, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent hydrogen, deuterium, halogen, halogen-substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or R₁₀₄ to R₁₀₇ may be linked to an adjacent substituent(s) to form a substituted or unsubstituted fused ring, e.g., a substituted or unsubstituted naphthyl, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₁₁ each independently represent hydrogen, deuterium, halogen, halogen-substituted or unsubstituted (C1-C30)alkyl, a substituted or unsubstituted (C3-C30)cycloalkyl, or a substituted or unsubstituted (C6-C30)aryl; or may be linked to an adjacent substituent(s) to form a substituted or unsubstituted fused ring; and
s represents an integer of 1 to 3.

Specifically, the specific examples of the dopant compound include the following, but are not limited thereto.

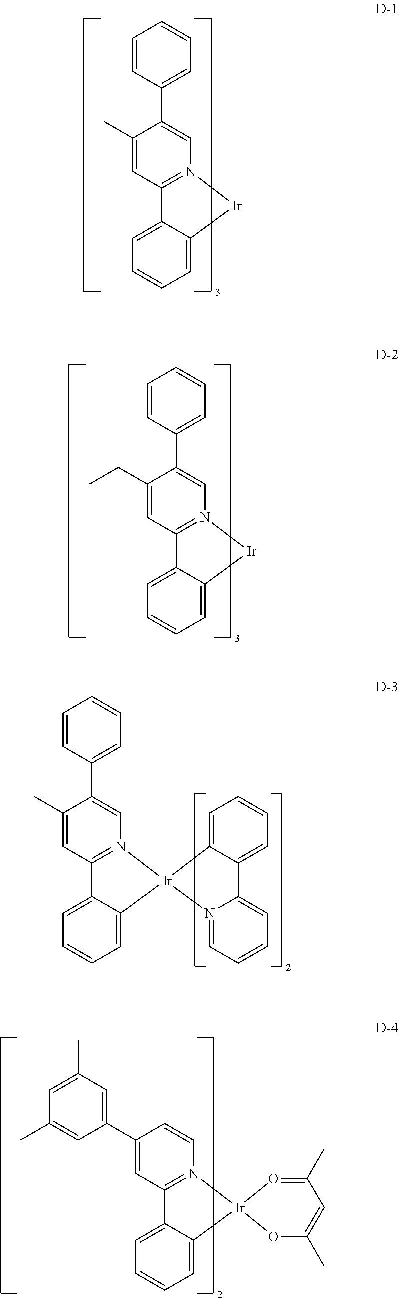

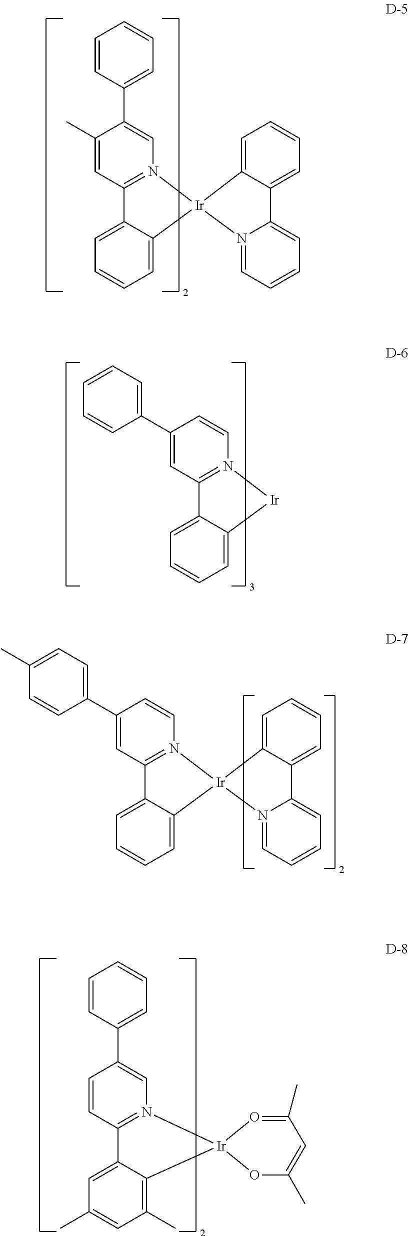

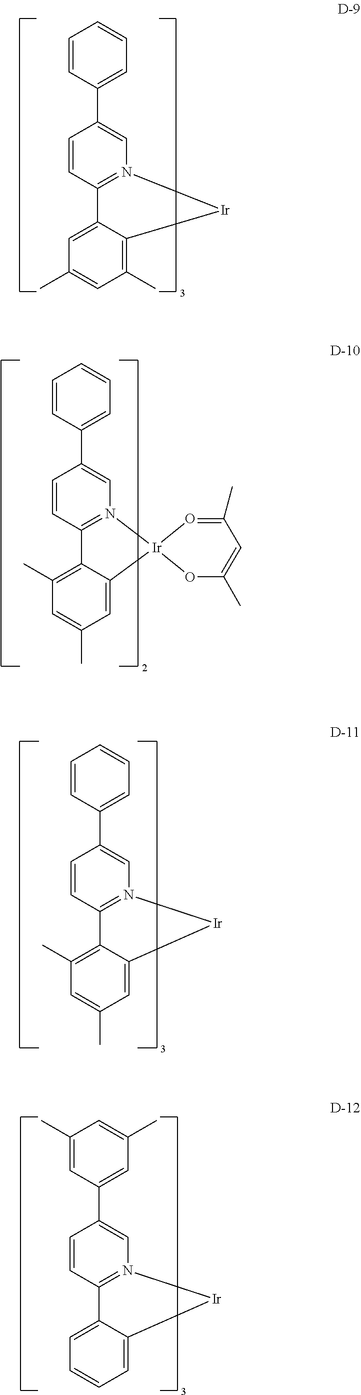

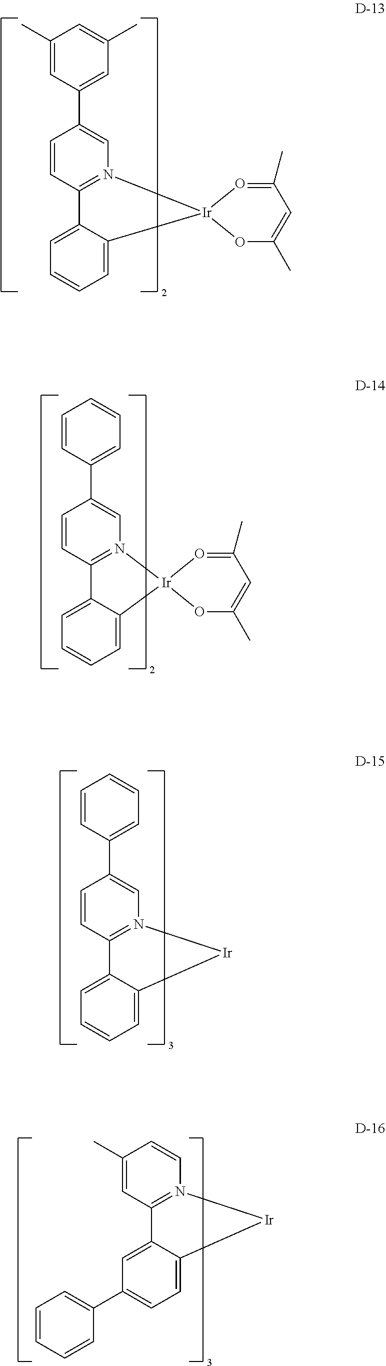

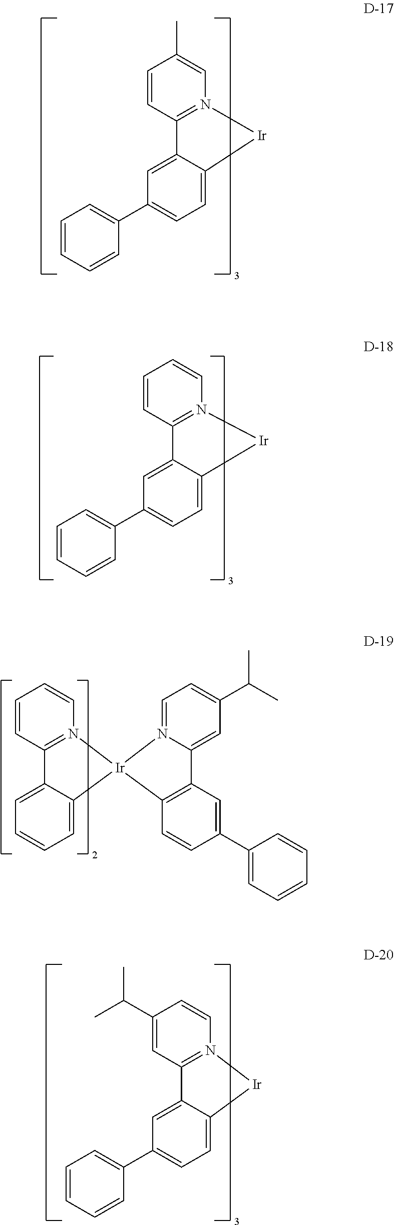

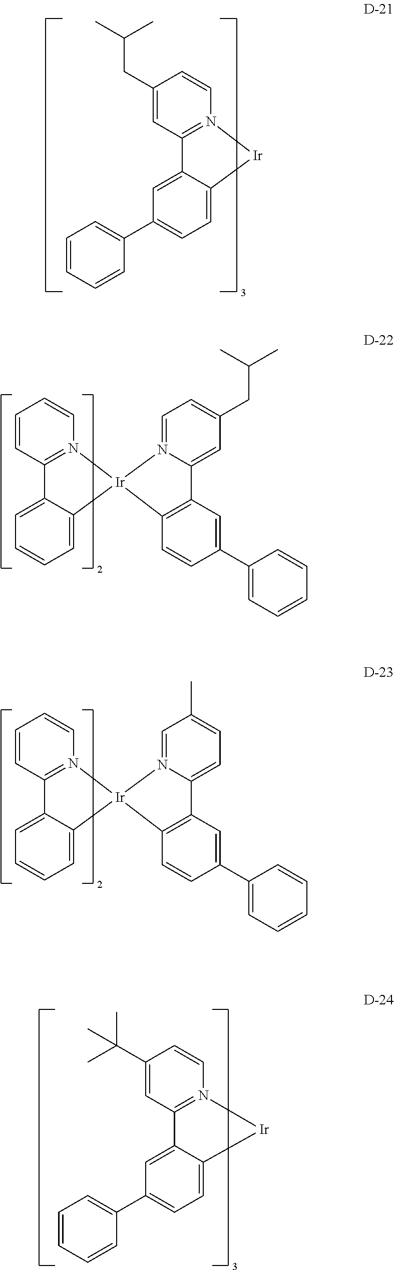

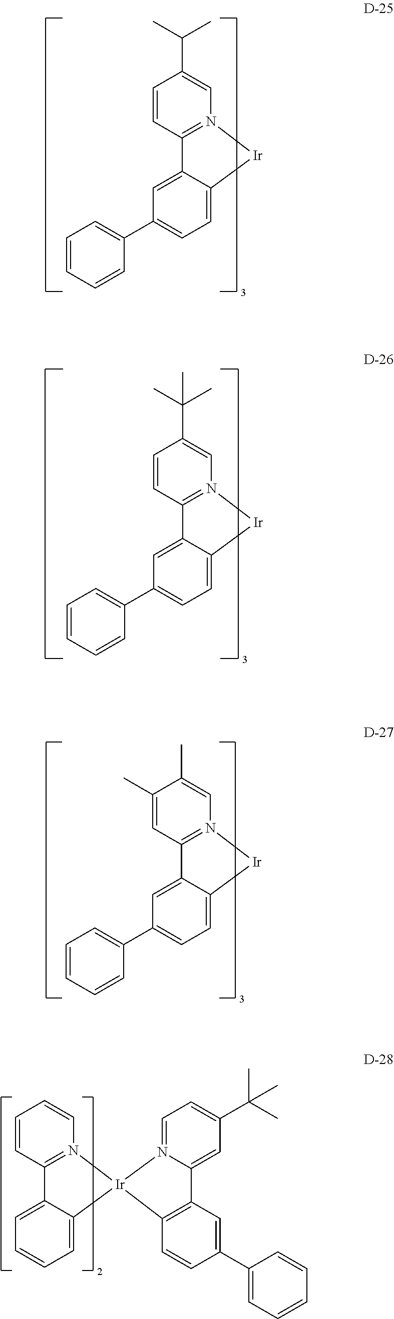

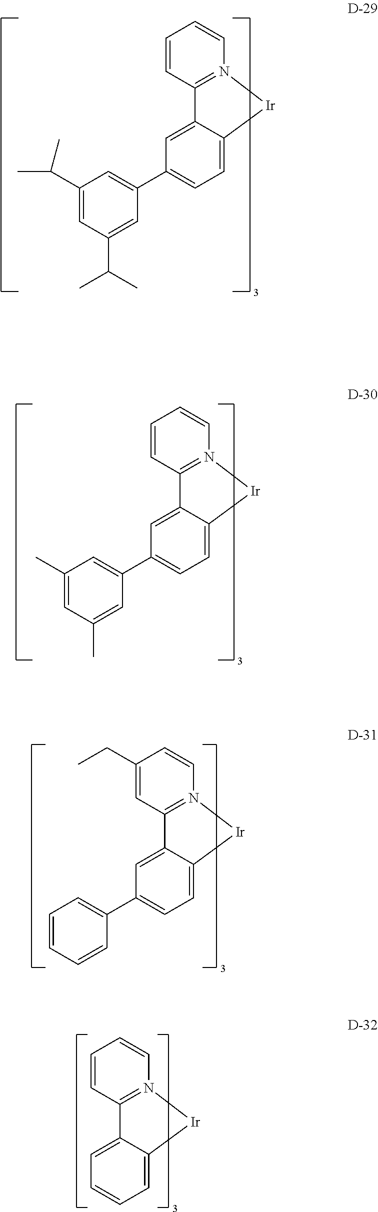

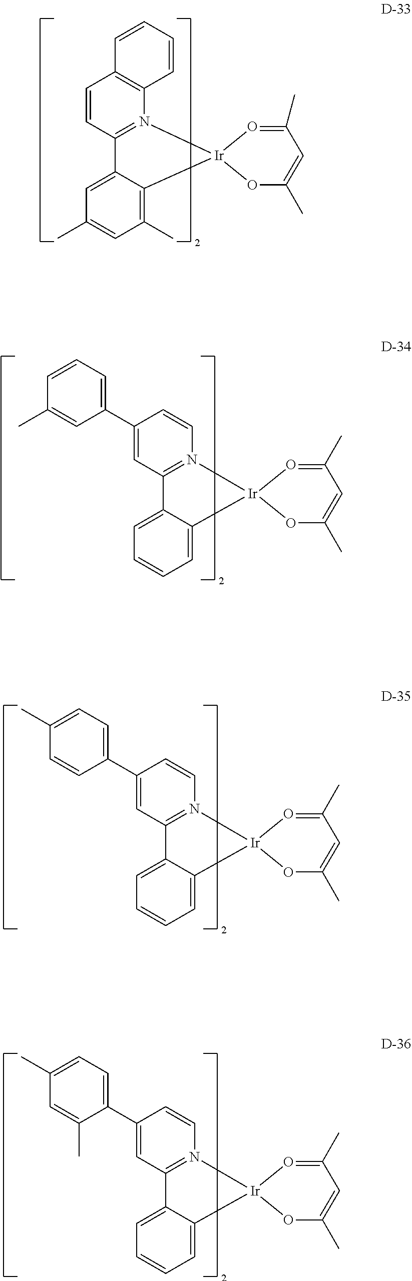

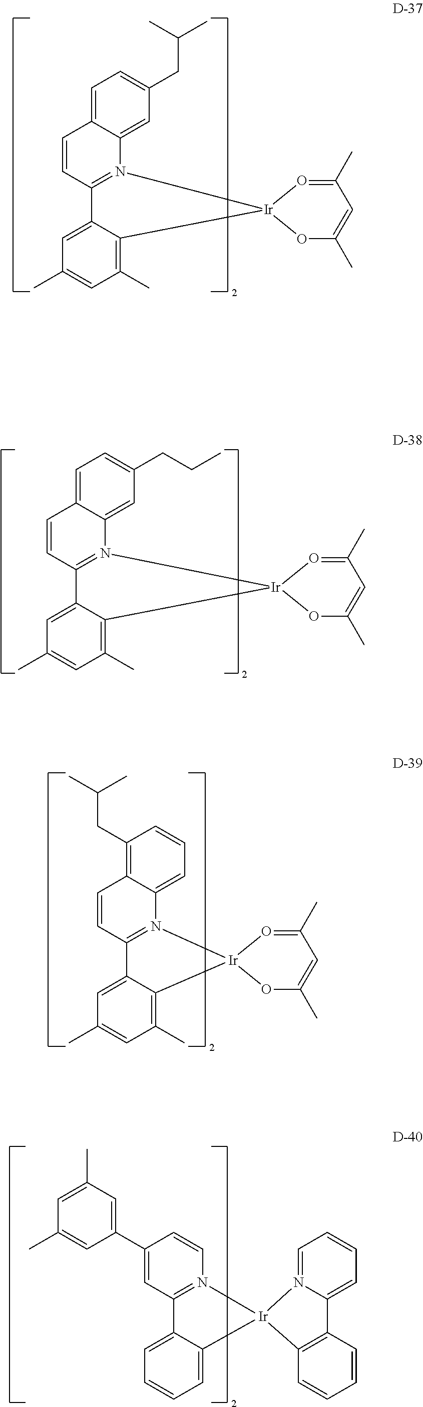

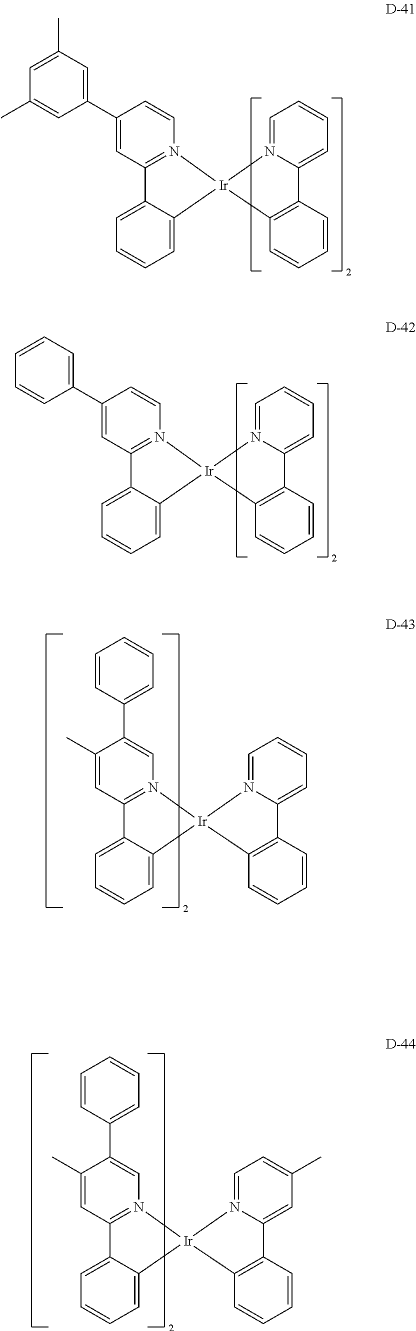

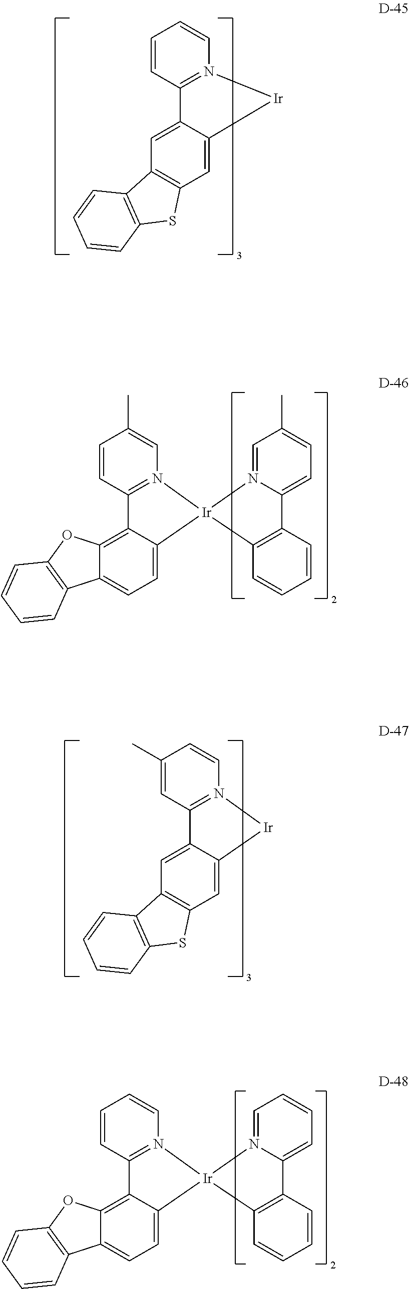

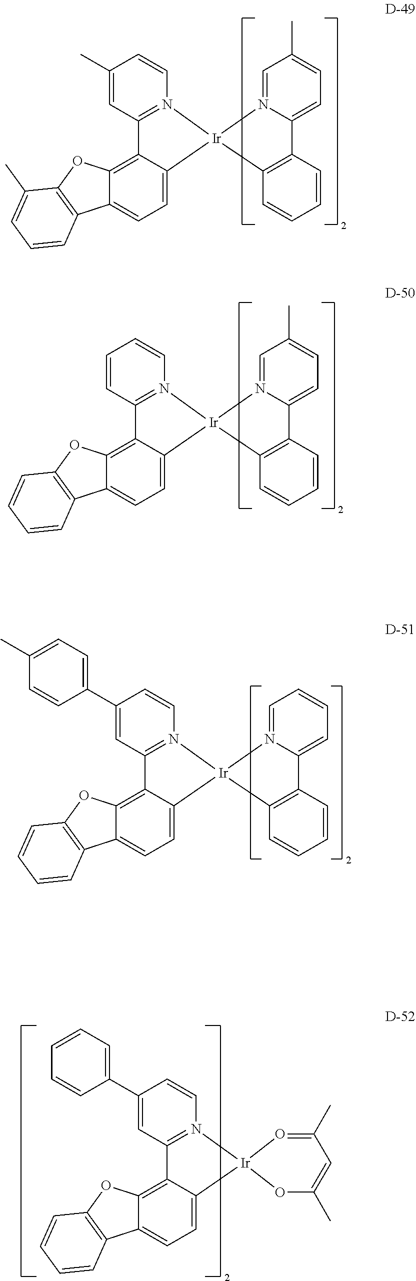

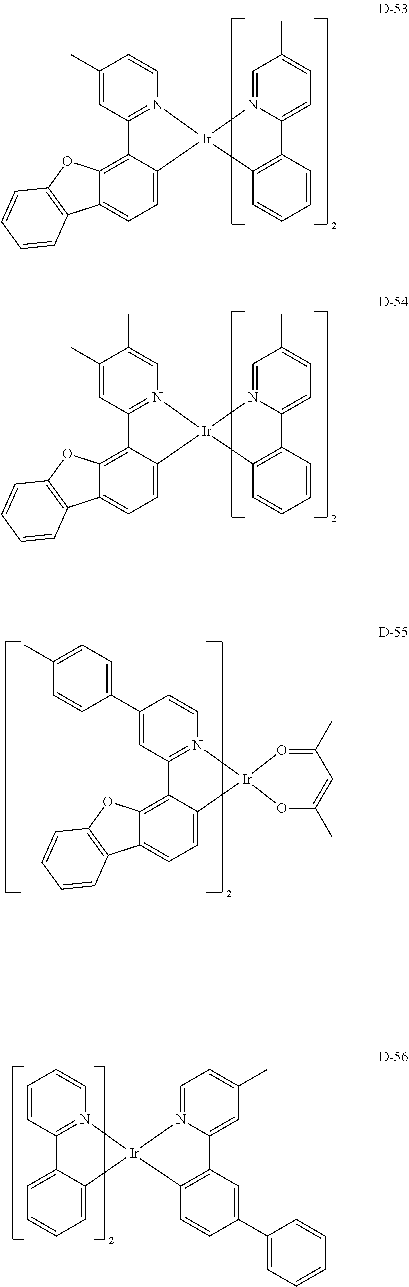

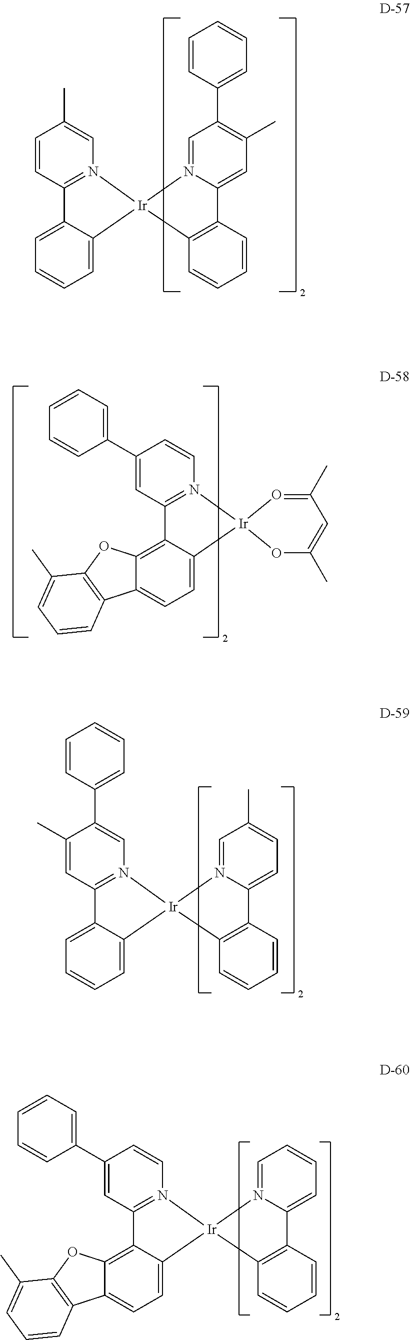

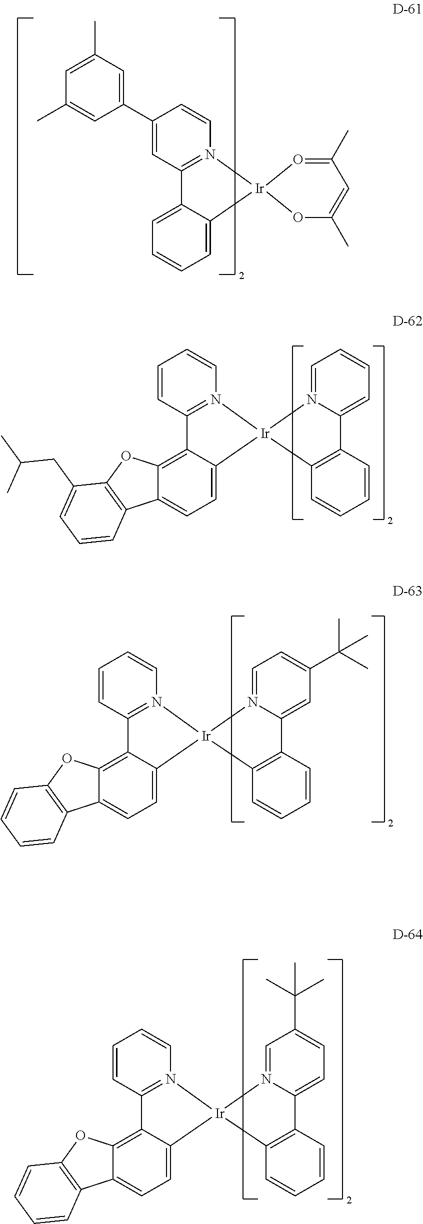

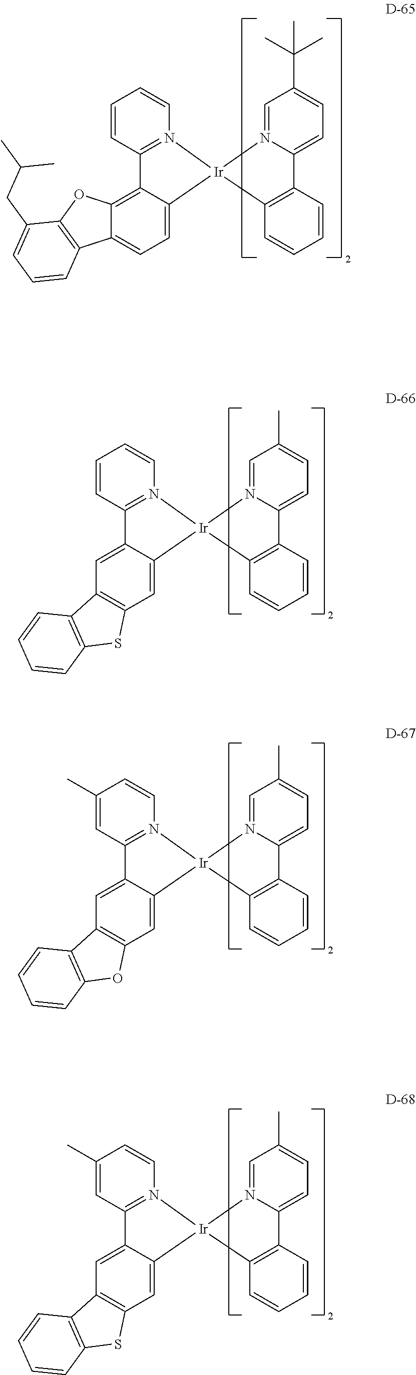

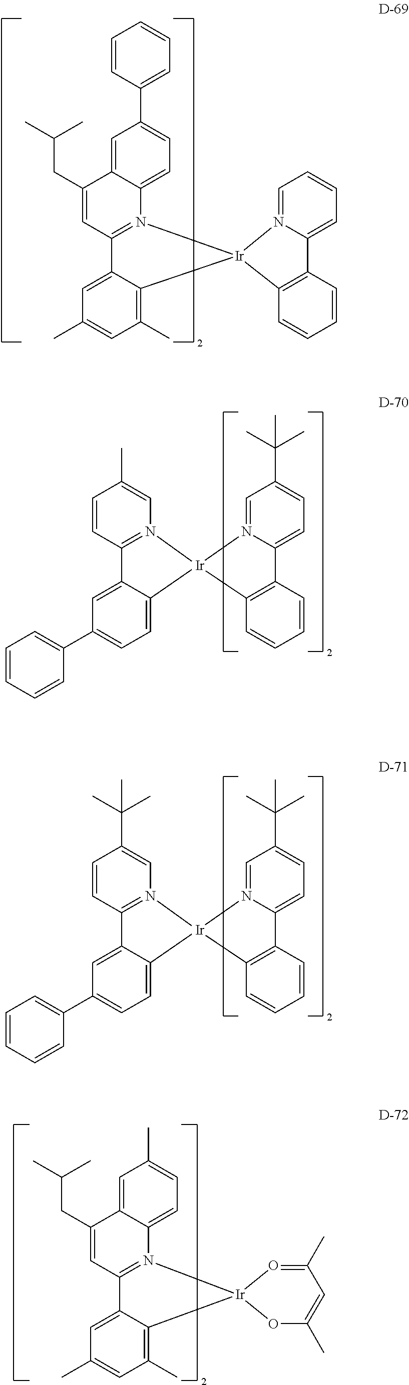

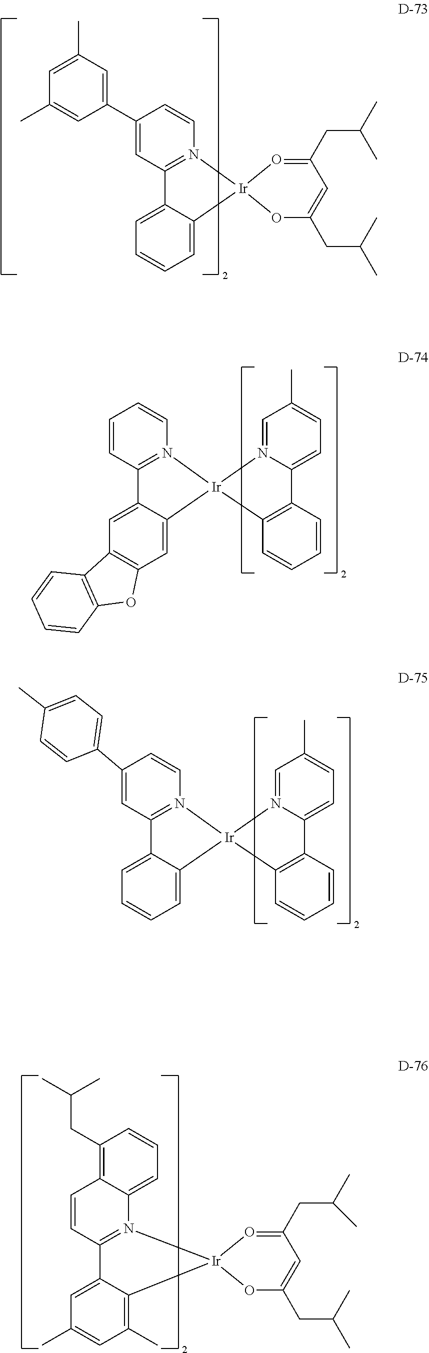

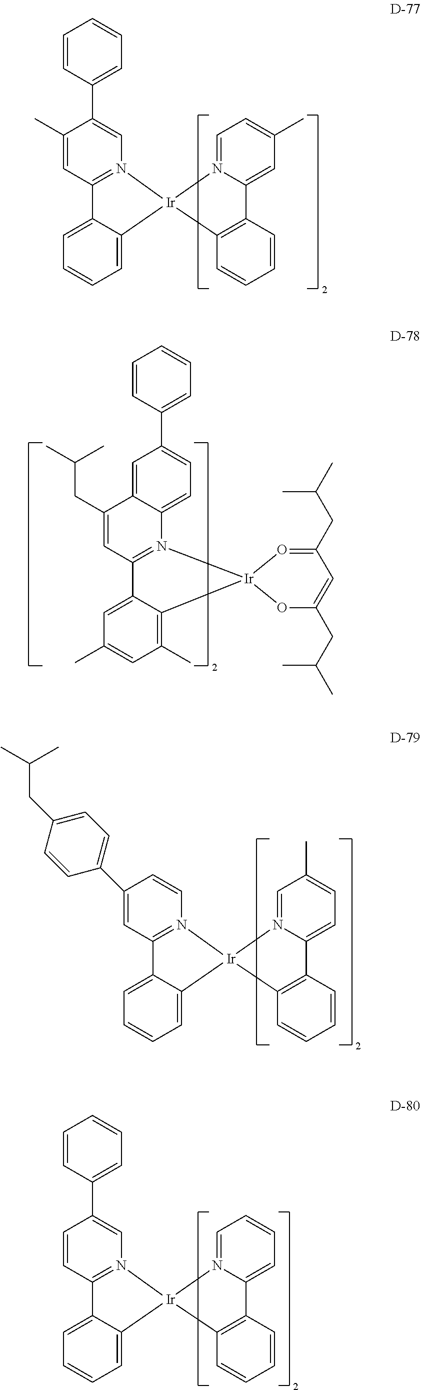

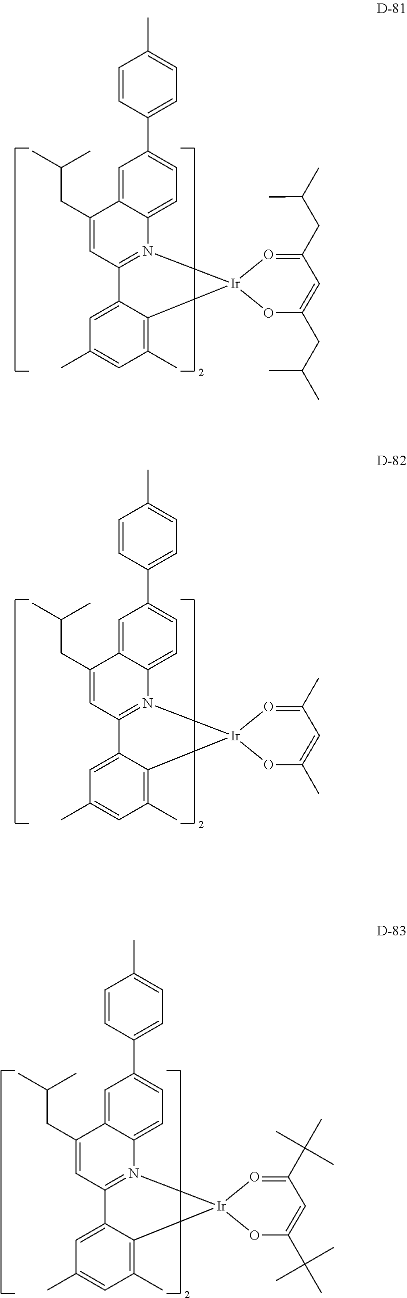

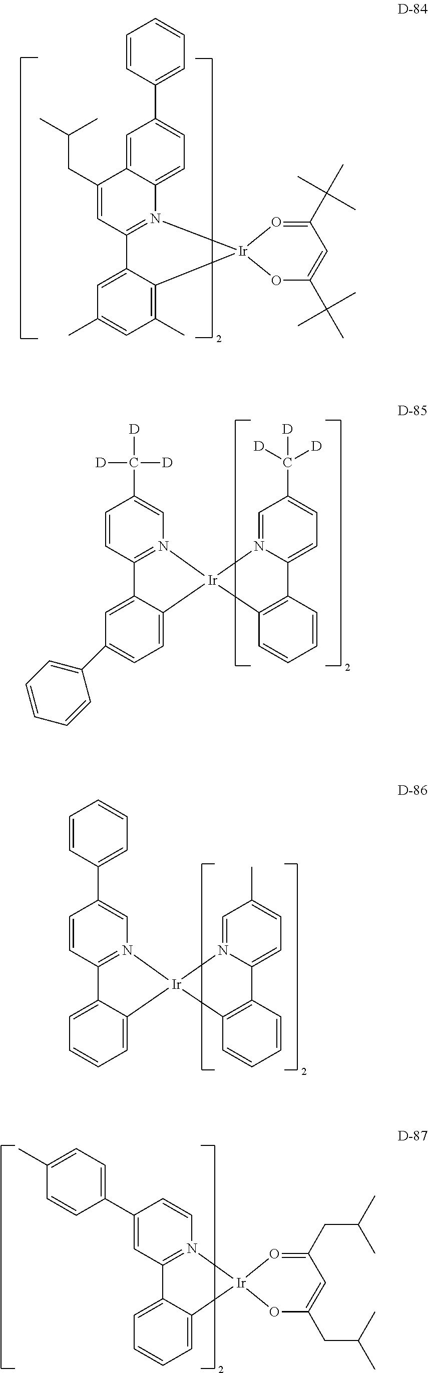

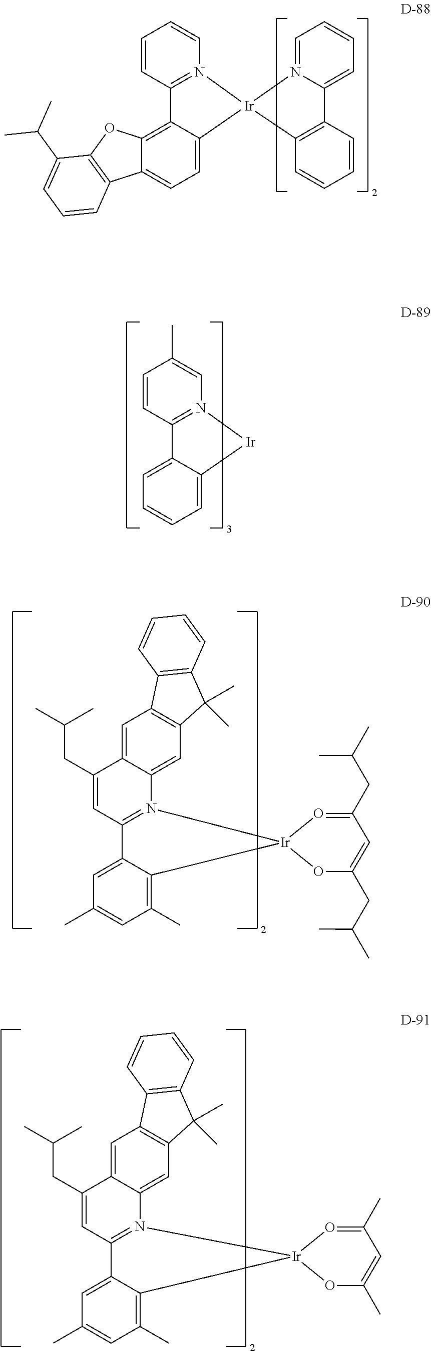

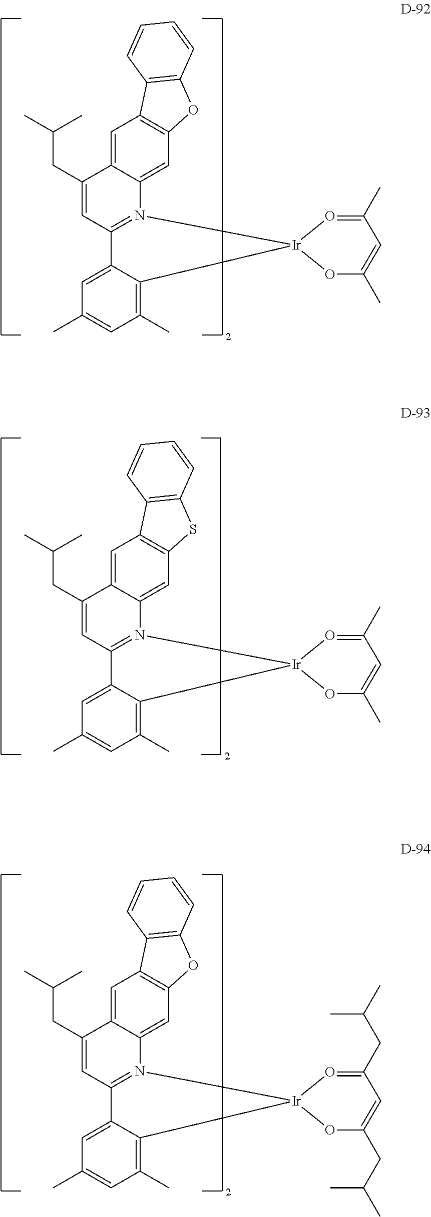

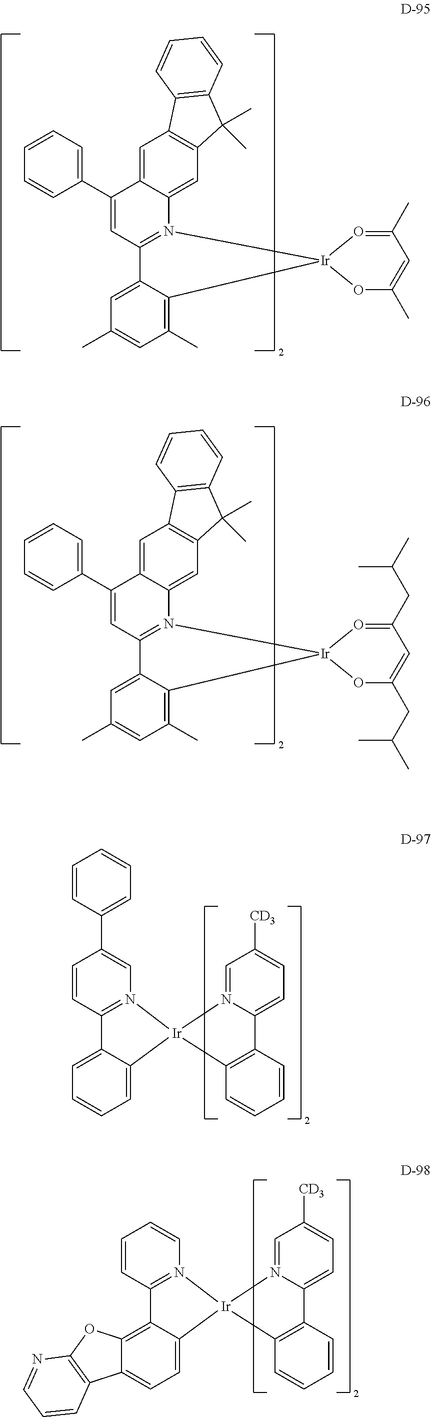

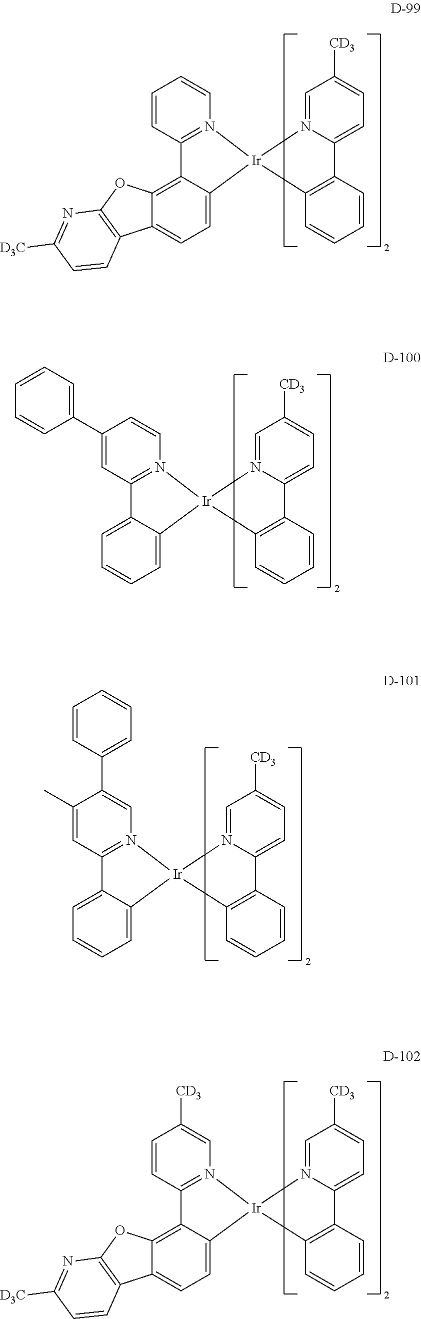

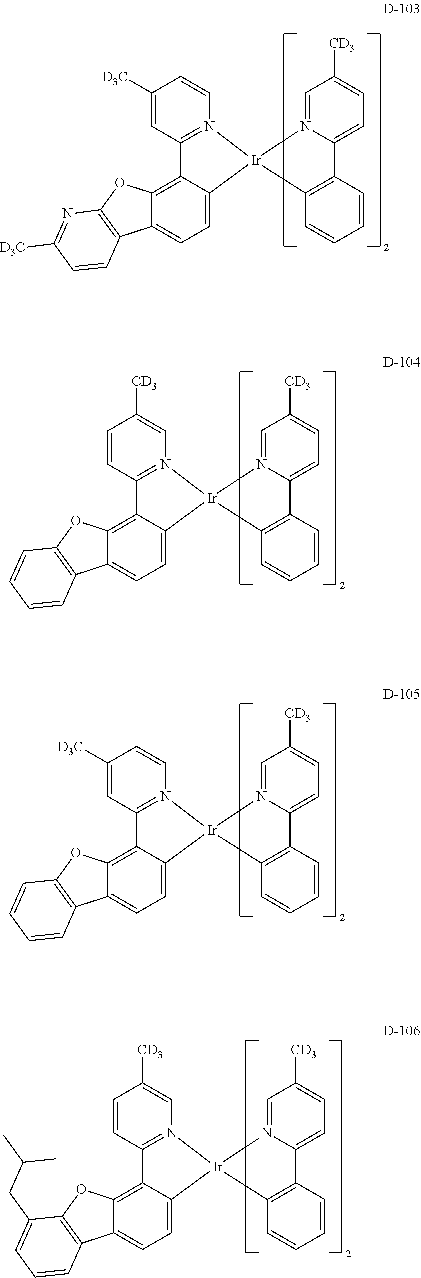

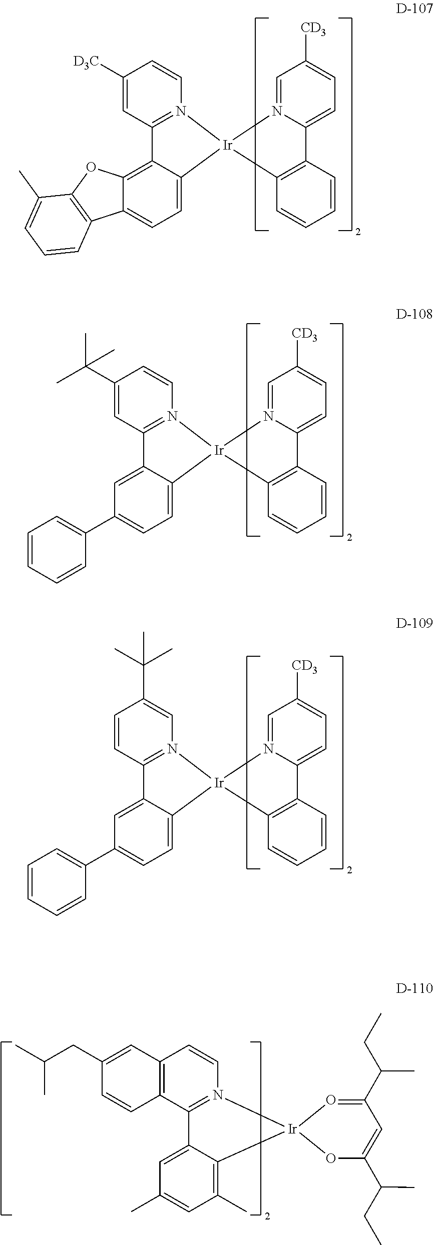

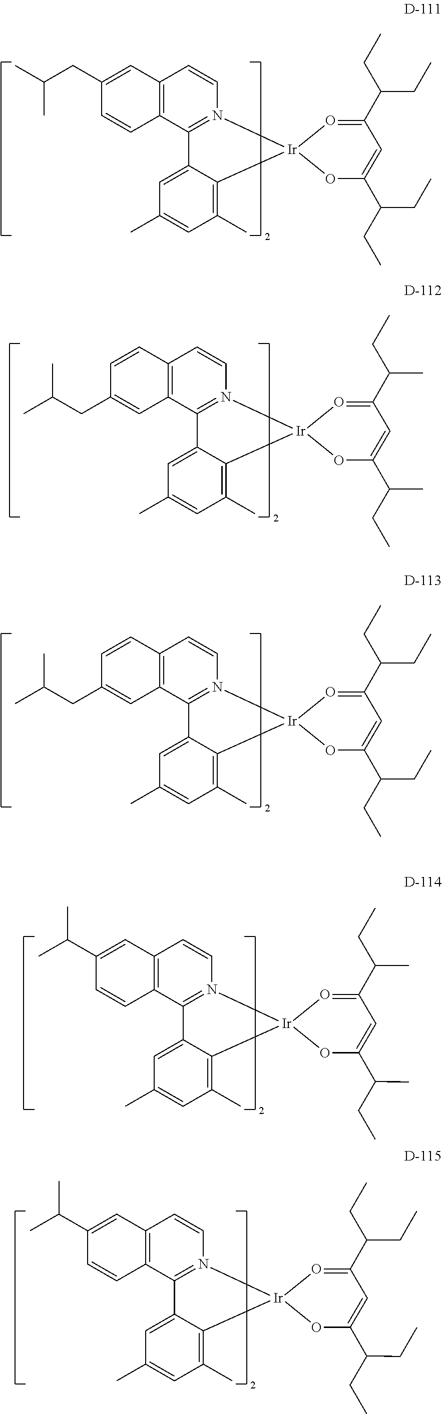

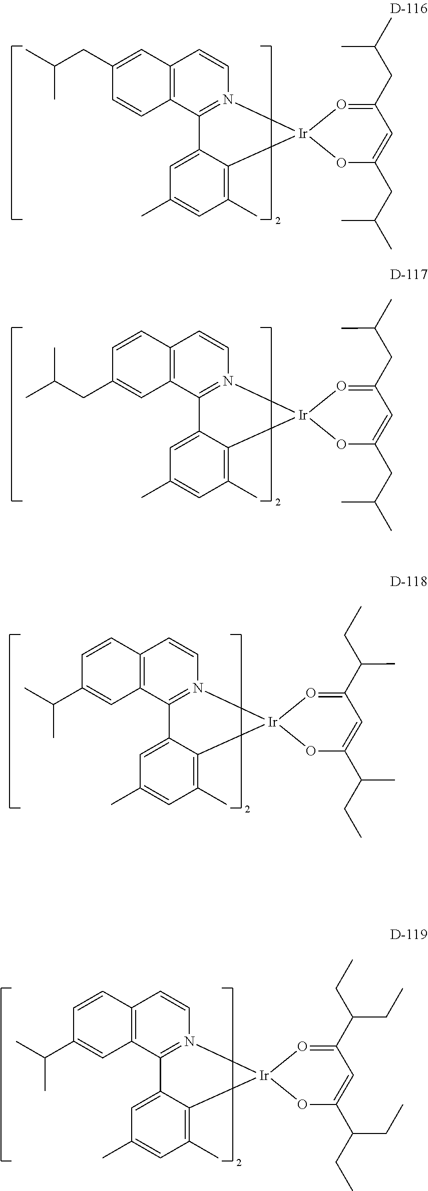

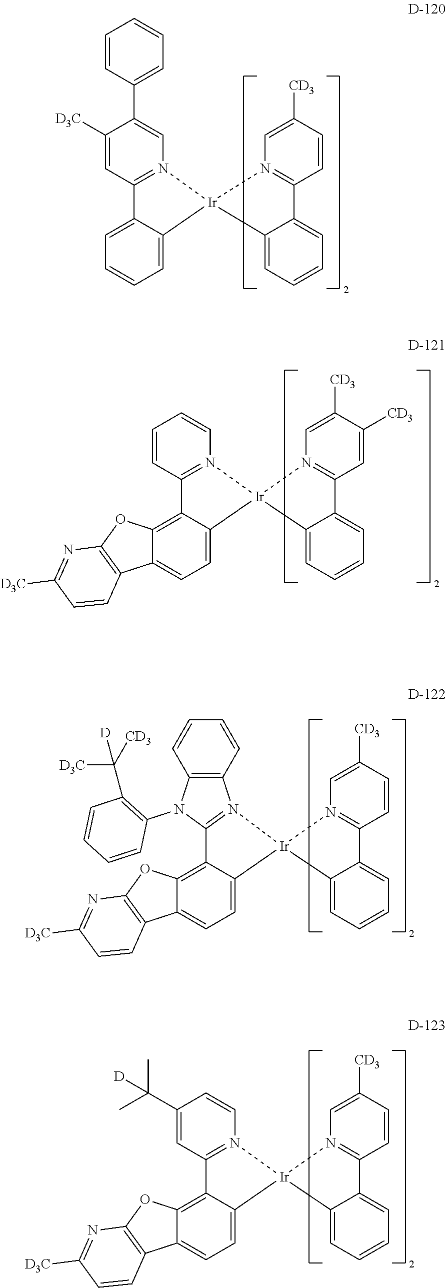

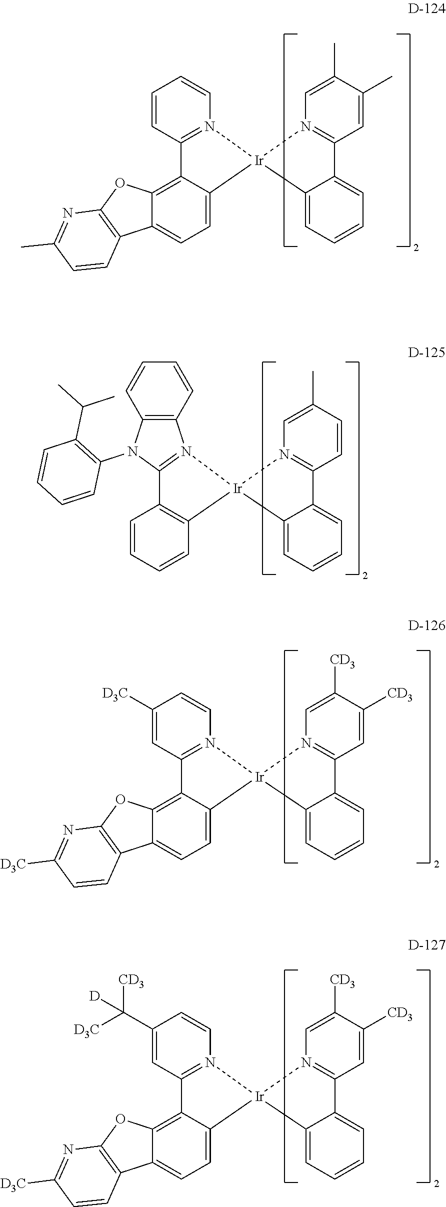

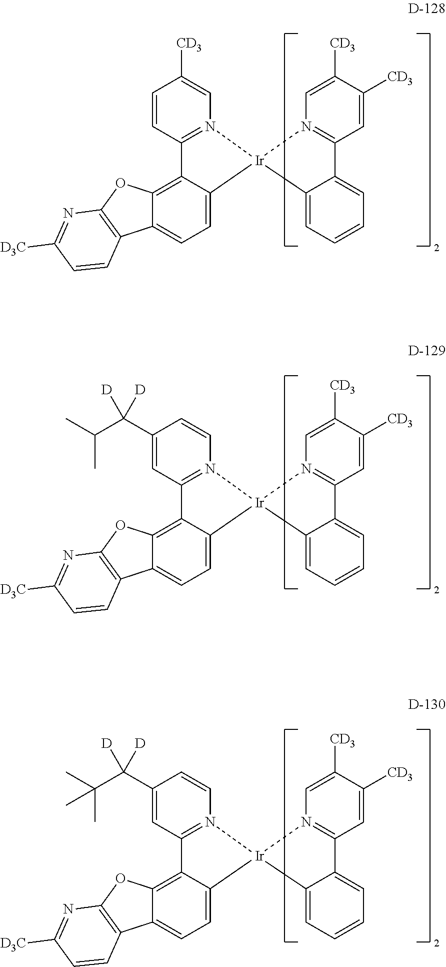

Hereinafter, the organic electroluminescent device to which the aforementioned organic electroluminescent compound or the organic electroluminescent material is applied will be described.

The organic electroluminescent device according to one embodiment includes a first electrode; a second electrode; and at least one organic layer interposed between the first electrode and the second electrode.

The compound represented by the formula 1 of the present disclosure may be included in at least one layer constituting the organic electroluminescent device. According to one embodiment, the organic layer includes a hole transport layer and/or a light-emitting layer comprising the organic electroluminescent compound according to the present disclosure. The hole transport layer and/or the light-emitting layer is comprised solely of the organic electroluminescent compound of the present disclosure or at least two species of the organic electroluminescent compound of the present disclosure, and may further comprise conventional materials included in the organic electroluminescent material.

In addition, the organic layer may comprise a hole transport layer and light-emitting layer, and may further comprise at least one layer selected from a hole injection layer, a hole auxiliary layer, a light-emitting auxiliary layer, an electron transport layer, an electron injection layer, an interlayer, a hole blocking layer, an electron blocking layer, and an electron buffer layer, wherein each layer may be further constituted of several layers. Also, the organic layer may further comprise at least one compound selected from the group consisting of an arylamine-based compound and a styryl arylamine-based compound, and may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

An organic electroluminescent material according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), B (blue), or YG (yellowish green) light-emitting units. In addition, the organic electroluminescent material according to one embodiment may also be applied to the organic electroluminescent device comprising a QD (quantum dot).

One of the first electrode and the second electrode may be an anode and the other may be a cathode, wherein the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. The electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both electrode(s). Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferable examples of the chalcogenide include SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferable examples of the oxidative dopant include various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

In order to form each layer of the organic electroluminescent device of the present disclosure, dry film-forming methods such as vacuum evaporation, sputtering, plasma, ion plating methods, etc., or wet film-forming methods such as inkjet printing, nozzle printing, slot coating, spin coating, dip coating, flow coating methods, etc., can be applied.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

Hereinafter, the preparation method of compounds according to the present disclosure will be explained with reference to the synthesis method of a representative compound or the intermediate compound of the present disclosure in order to understand the present disclosure in detail.

### [Example 1] Preparation of Compound C-34

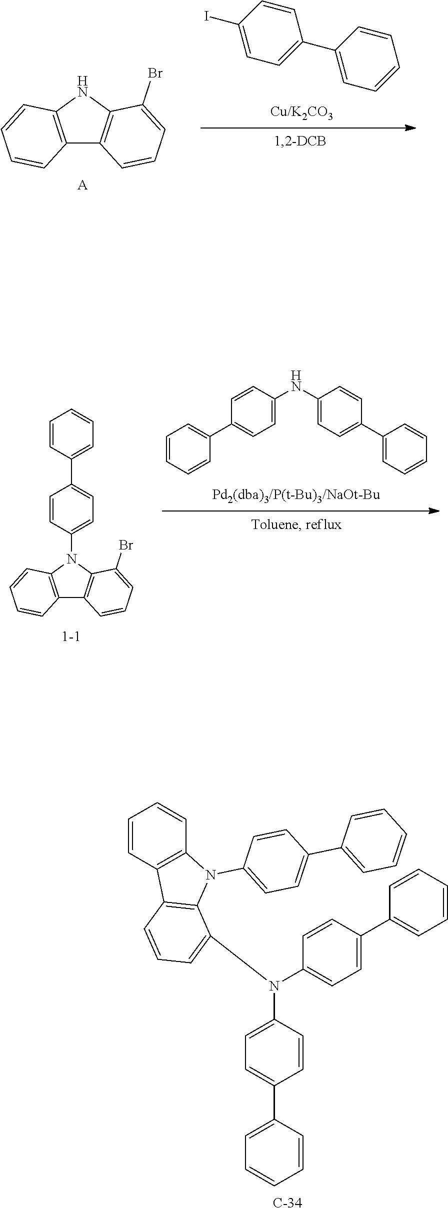

### 1) Synthesis of Compound 1-1

Compound A (20 g, 81.27 mmol), 4-iodo-1,1′-biphenyl (34 g, 121.90 mmol), copper powder (Cu) (2.6 g, 40.64 mmol), potassium carbonate (K₂CO₃) (22.5 g, 162.54 mmol), and 406 mL of 1,2-dichlorobenzene (1,2-DCB) were added into a reaction vessel and stirred at 200° C. for 24 hours. After completion of the reaction, the organic layer mixture was washed with distilled water and extracted with ethyl acetate. The extracted organic layer was dried with magnesium sulfate and the solvent was removed by a rotary evaporator. Thereafter, the residue was purified by column chromatography to obtain the compound 1-1 (19 g, yield: 59%).

### 2) Synthesis of Compound C-34

Compound 1-1 (5.0 g, 12.55 mmol), di([1,1′-biphenyl]-4-yl)amine (4.4 g, 13.81 mmol), tris(dibenzylideneacetone)dipalladium(0) (Pd₂(dba)₃) (0.6 g, 0.63 mmol), tri-t-butylphosphine (P(t-Bu)₃) (0.6 mL, 1.26 mmol), sodium tert-butoxide (NaOt-Bu) (1.8 g, 18.83 mmol), and 63 mL of toluene were added into a reaction vessel and refluxed for 1 hour. After cooling the reacting mixture to room temperature, the solid was filterated and washed off with ethyl acetate. The remaining liquid was distilled under reduced pressure and purified by column chromatography to obtain compound C-34 (2.1 g, yield: 26%).

| | MW | M.P |
|---|---|---|
| C-34 | 638.80 | 225° C. |

### [Example 2] Preparation of Compound C-36

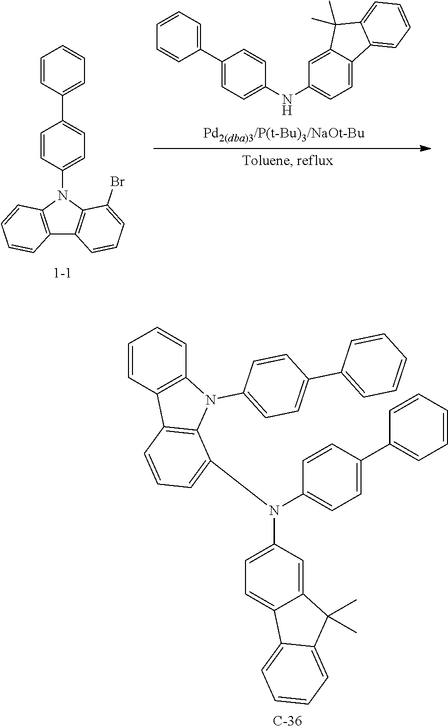

Compound 1-1 (3.0 g, 11.16 mmol), N-(1,1′-biphenyl-4-yl)-9,9-dimethyl-9H-fluorene-2-amine (5.0 g, 12.28 mmol), Pd₂(dba)₃ (0.5 g, 0.56 mmol), P(t-Bu)₃ (0.3 mL, 1.12 mmol), NaOt-Bu (1.6 g, 16.74 mmol), and 56 mL of toluene were added into a reaction vessel and refluxed for 1 hour. After cooling the reacting mixture to room temperature, the solid was filterated and washed off with ethyl acetate. The remaining liquid was distilled under reduced pressure and purified by column chromatography to obtain compound C-36 (2 g, yield: 28%).

| | MW | M.P |
|---|---|---|
| C-36 | 678.86 | 168° C. |

Hereinafter, the properties of an organic electroluminescent device comprising an organic electroluminescent compound will be explained in order to understand the present disclosure in detail.

### [Device Examples 1 to 7] Producing OLEDs Comprising the Organic Electroluminescent Compound According to the Present Disclosure

OLEDs comprising the organic electroluminescent compound according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropanol, sequentially, and then was stored in isopropanol. Next, the ITO substrate was mounted on a substrate holder of a vacuum vapor deposition apparatus. Compound HI-1 was introduced into a cell of the vacuum vapor deposition apparatus, and the pressure in the chamber of the apparatus was then controlled to 10⁻⁶ torr. Thereafter, an electric current was applied to the cell to evaporate the introduced material, thereby forming a first hole injection layer having a thickness of 80 nm on the ITO substrate. Compound HI-2 was then introduced into another cell of the vacuum vapor deposition apparatus, and an electric current was applied to the cell to evaporate the introduced material, thereby forming a second hole injection layer having a thickness of 5 nm on the first hole injection layer. Next, compound HT-1 was introduced into another cell of the vacuum vapor deposition apparatus. Thereafter, an electric current was applied to the cell to evaporate the introduced material, thereby forming a first hole transport layer having a thickness of 10 nm on the second hole injection layer. The compound listed in the following Table 2 as a second hole transport material was then introduced into another cell of the vacuum vapor deposition apparatus, and an electric current was applied to the cell to evaporate the introduced material, thereby forming a second hole transport layer having a thickness of 30 nm on the first hole transport layer. After forming the hole injection layers and the hole transport layers, a light-emitting layer using a plurality of host materials was then deposited thereon as follows: The compound H-1 as a host was introduced into one cell of the vacuum vapor deposition apparatus and compound H-2 was introduced into another cell as a host. The two host materials were evaporated at a rate of 2:1 and, at the same time, the compound D-99 was introduced into another cell as a dopant. The dopant was doped in a doping amount of 10 wt % with respect to the rate of the deposition of the light-emitting layer, to form a light-emitting layer having a thickness of 40 nm on the hole transport layer. Next, compounds ET-1 and El-1 were introduced into another cell, were evaporated at a rate of 1:1, and were deposited to form an electron transport layer having a thickness of 35 nm on the light-emitting layer. After depositing compound El-1 as an electron injection layer having a thickness of 2 nm on the electron transport layer, an A1 cathode having a thickness of 800 nm was deposited on the electron injection layer by another vacuum vapor deposition apparatus. Thus, OLEDs were produced.

### [Comparative Example 1] Producing an OLED Comprising the Conventional Compound

An OLED was produced in the same manner as in Device Examples 1 to 7, except that compound G′-1 was used as in a second host transport layer.

The compounds used in Device Examples 1 to 7 and Comparative Example 1 are shown specifically in Table 1 below.

TABLE 1

| | | |
|---|---|---|
| Hole Injection Layer/ Hole Transport Layer | 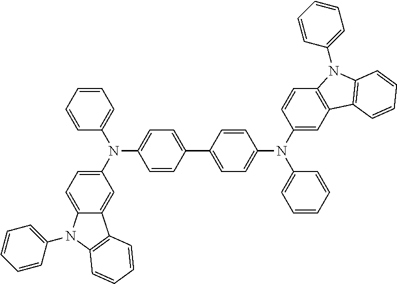 | HI-1 |
| | 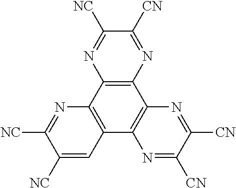 | HI-2 |
| | 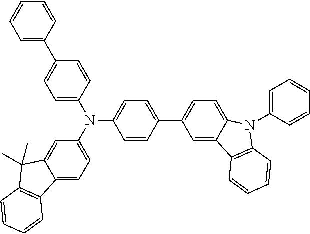 | HT-1 |
| | 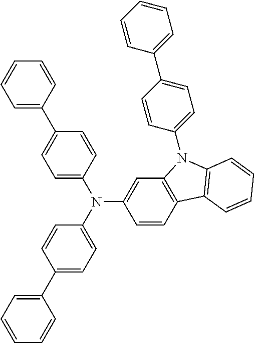 | G′-1 |
| | 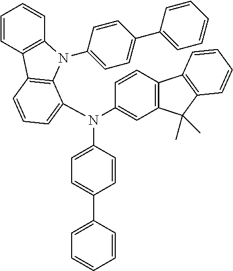 | C-36 |
| | 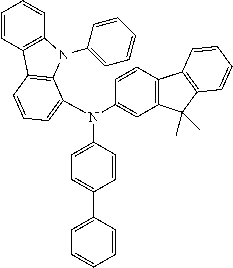 | C-5 |
| | 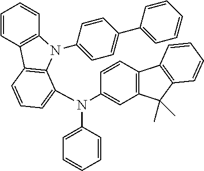 | C-35 |
| | 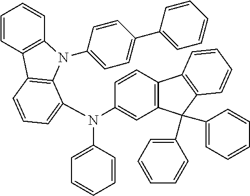 | C-140 |
| | 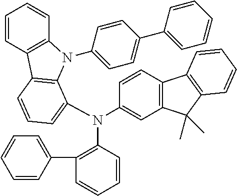 | C-38 |
| | 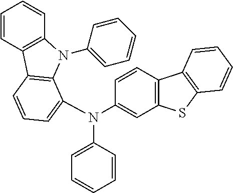 | C-28 |
| | 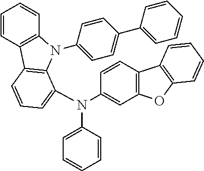 | C-49 |
| Light- Emitting Layer | 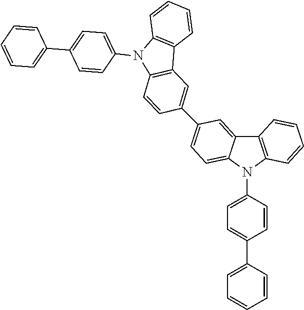 | H-1 |
| | 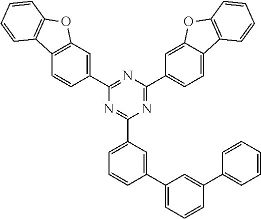 | H-2 |
| | 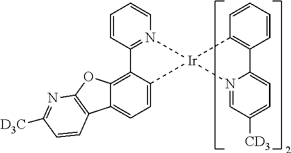 | D-99 |
| Electron Transport Layer/ Electron Injection Layer | 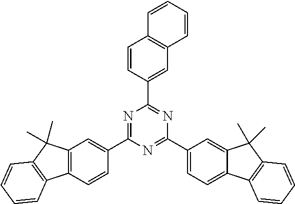 | ET-1 |
| | 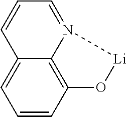 | EI-1 |

The results of the driving voltage, the current efficiency, the power efficiency, the external quantum efficiency, and the color coordinates at a luminance of 1,000 nits, of the organic electroluminescent device of Device Examples 1 to 7 and Comparative Example 1 produced as described above, are shown in the following Table 2.

| | Second | | | | External | Color | |
|---|---|---|---|---|---|---|---|
| | Host | Driving | Current | Power | Quantum | Coordinates | |
| | Transport | Voltage | Efficiency | Efficiency | Efficiency | CIE | |
| | Material | (V) | (Cd/A) | (Lm/W) | (%) | x | y |
| Device | C-36 | 2.6 | 98.4 | 117.1 | 25.3 | 0.336 | 0.636 |
| Example 1 | | | | | | | |
| Device | C-5 | 2.7 | 97.0 | 114.5 | 25.0 | 0.338 | 0.634 |
| Example 2 | | | | | | | |
| Device | C-35 | 2.7 | 101.8 | 120.4 | 26.2 | 0.336 | 0.636 |
| Example 3 | | | | | | | |
| Device | C-140 | 2.7 | 96.8 | 114.1 | 24.8 | 0.334 | 0.638 |
| Example 4 | | | | | | | |
| Device | C-38 | 2.6 | 95.3 | 113.9 | 24.4 | 0.335 | 0.637 |
| Example 5 | | | | | | | |
| Device | C-28 | 2.6 | 100.3 | 118.9 | 25.7 | 0.335 | 0.637 |
| Example 6 | | | | | | | |
| Device | C-49 | 2.6 | 99.8 | 118.8 | 25.6 | 0.333 | 0.638 |
| Example 7 | | | | | | | |
| Comparative | G'-1 | 2.7 | 93.0 | 110.3 | 23.9 | 0.338 | 0.634 |
| Example 1 | | | | | | | |

1. An organic electroluminescent compound represented by the following formula 1: 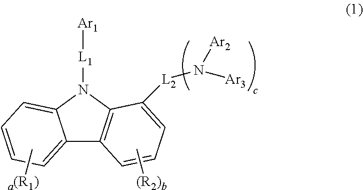
wherein,
Ar₁ to Ar₃ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
L₁ and L₂ each independently represent a single bond, a substituted or unsubstituted (C6-C30)arylene, or a substituted or unsubstituted (3- to 30-membered)heteroarylene;
R₁ and R₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
a represents an integer of 1 to 4, b represents an integer of 1 to 3, c represents an integer of 1 or 2, provided that when L₂ is a single bond, c is 1; and
when a, b, and c are 2 or more, each of R₁, each of R₂, and each of —NAr₂ Ar₃ may be the same or different.

2. The organic electroluminescent compound according to claim 1, wherein the formula 1 is represented by the following formula 2 or 3: 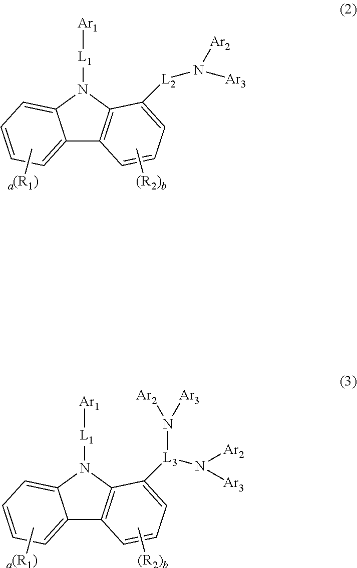
wherein,
Ar₁ to Ar₃, L₁, L₂, R₁ and R₂, a, and b are as defined in claim 1, and
L₃ represents a substituted or unsubstituted (C6-C30)arylene or a substituted or unsubstituted (3- to 30-membered)heteroarylene.

3. The organic electroluminescent compound according to claim 1, wherein Ar₂ and Ar₃ each independently represent any one of the substituents selected from the following Group 1: 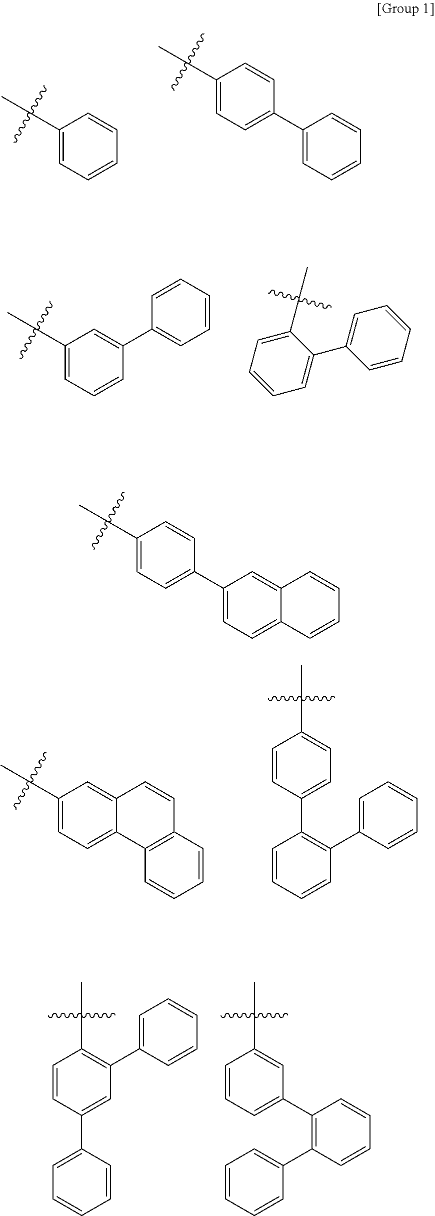 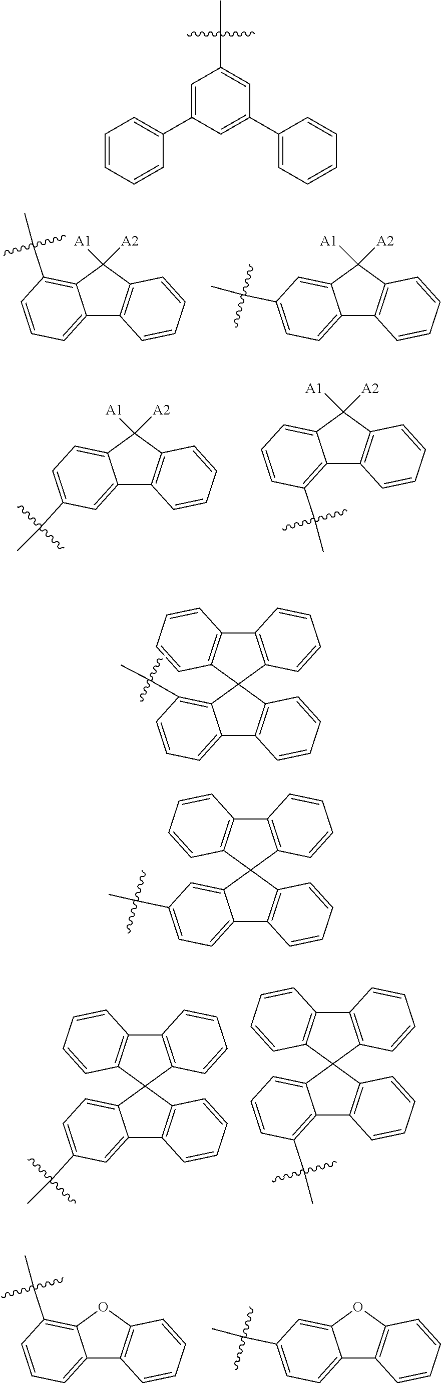 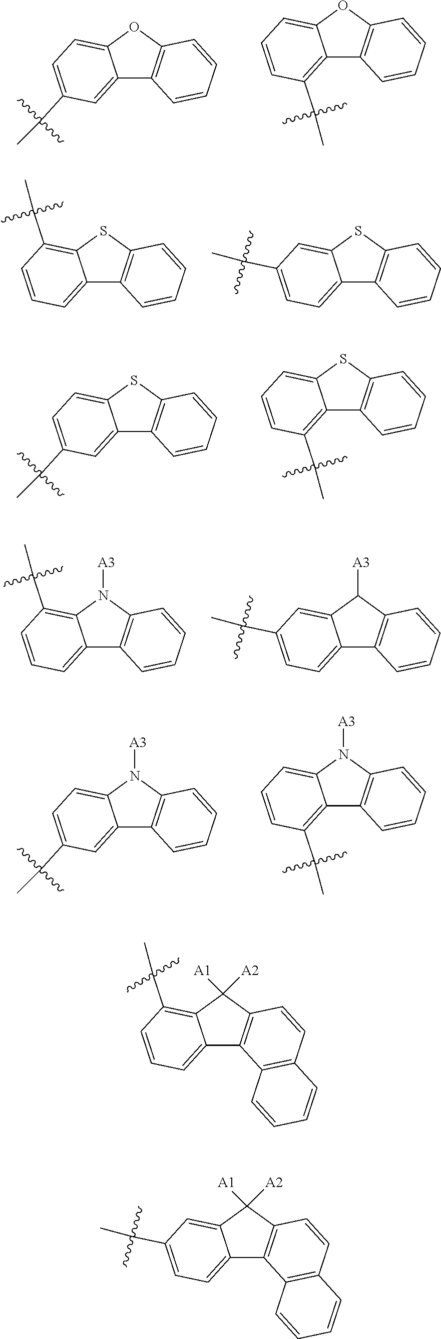 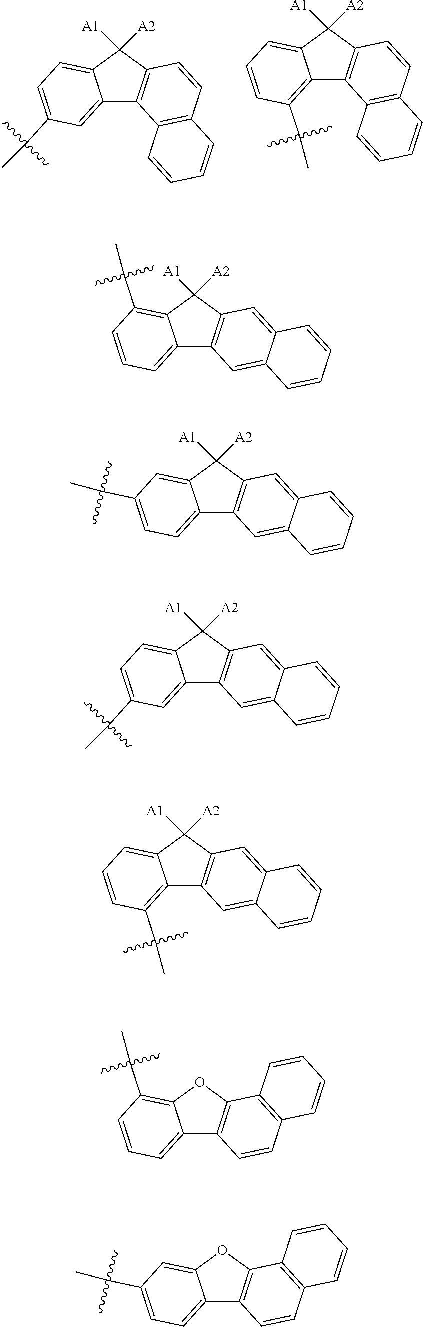 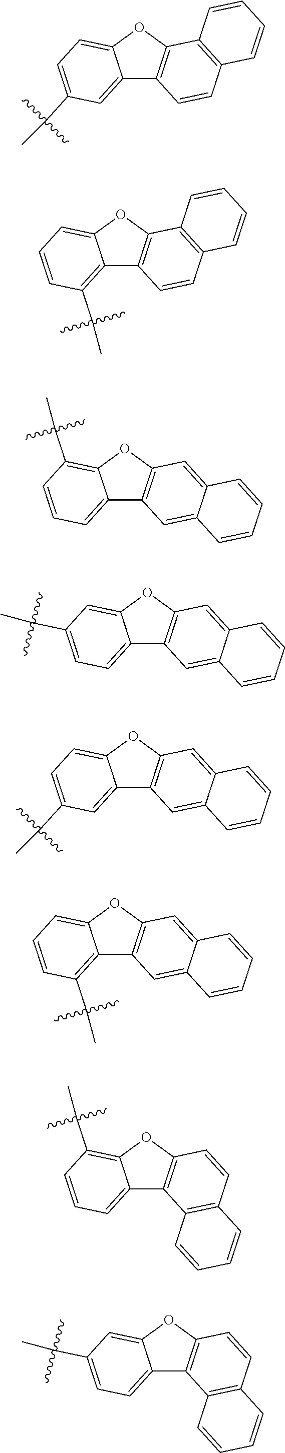 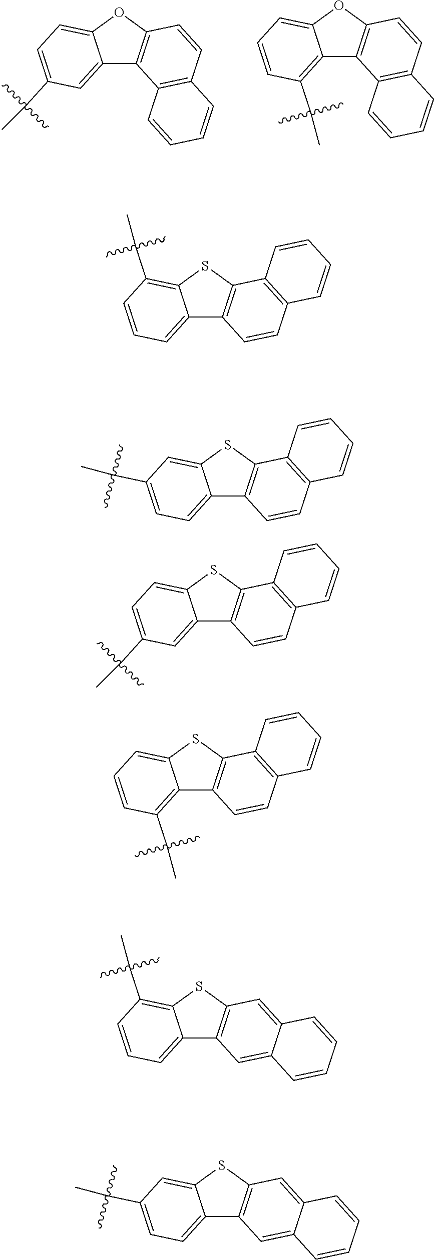 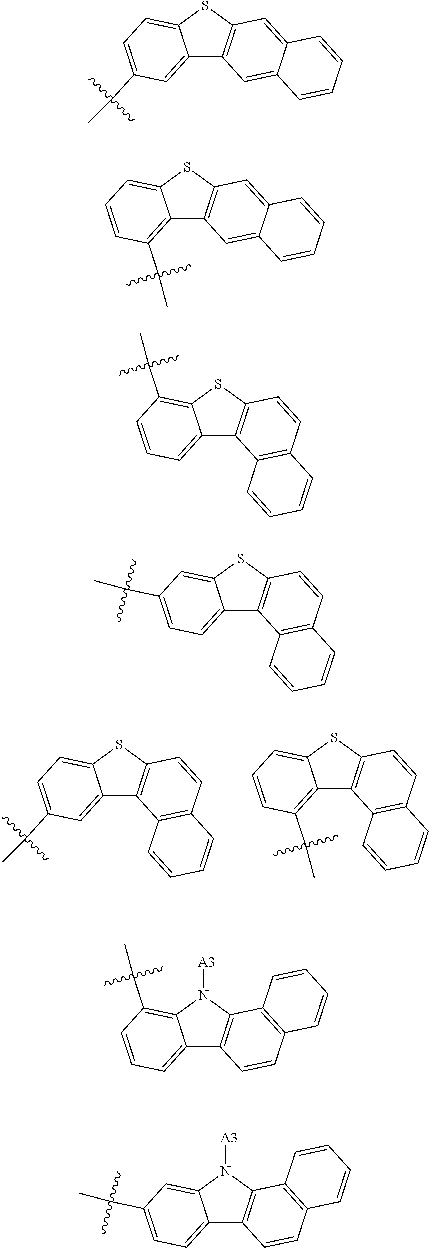 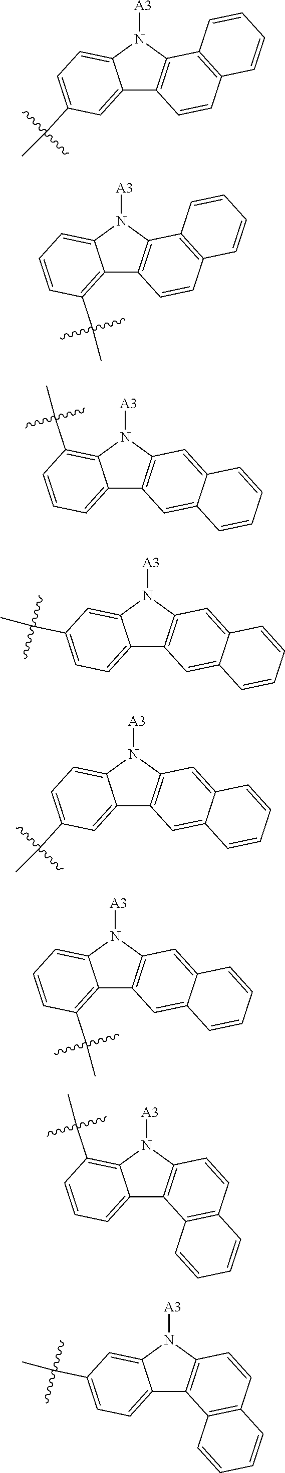 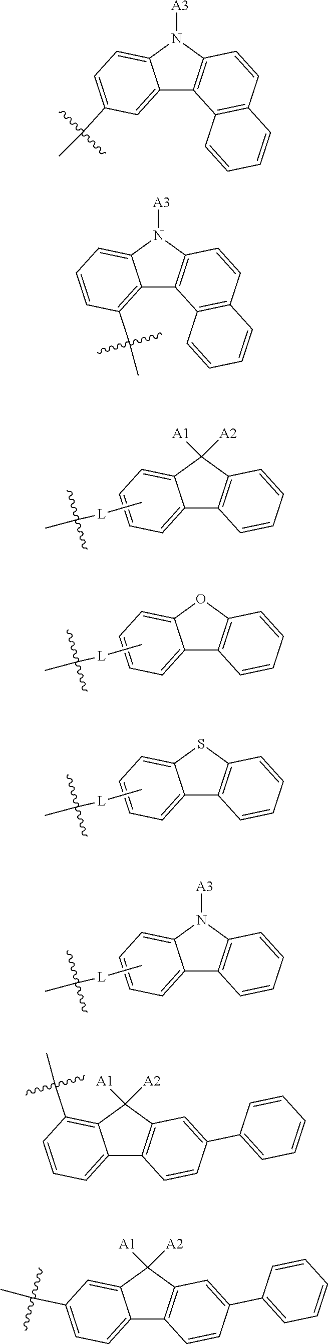 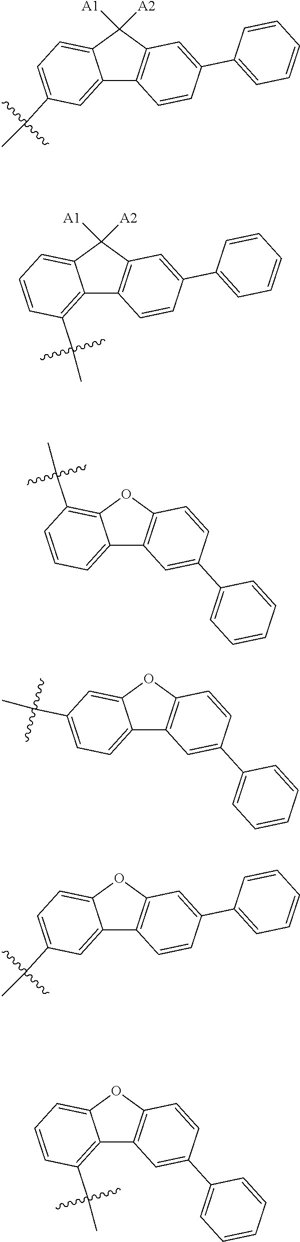 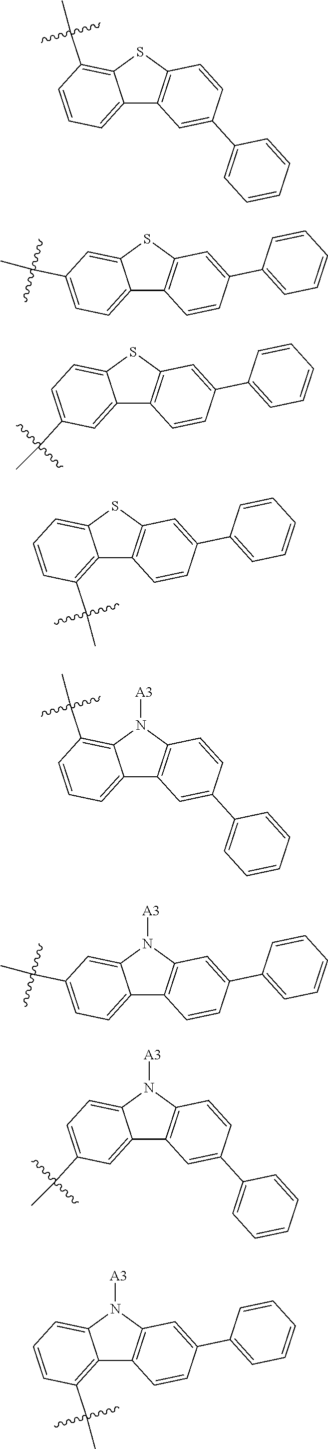 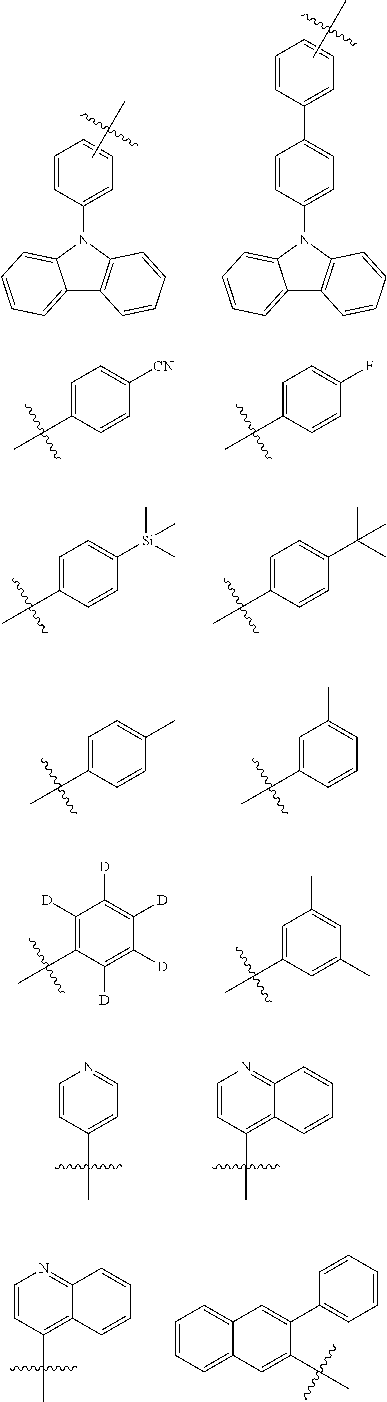
wherein,
A1 to A3 each independently represent a substituted or unsubstituted (C1-C30)alkyl, a substituted or unsubstituted (C6-C30)aryl, or a substituted or unsubstituted (3- to 30-membered)heteroaryl; and
L represents a substituted or unsubstituted (C6-C30)arylene or a substituted or unsubstituted (3- to 30-membered)heteroarylene.

4. The organic electroluminescent compound according to claim 1, wherein Ar₁ to Ar₃ each independently represent a substituted or unsubstituted (C6-C30)aryl or a substituted or unsubstituted (5- to 25-membered)heteroaryl;
L₁ and L₂ each independently represent a single bond, a substituted or unsubstituted (C6-C25)arylene, or a substituted or unsubstituted (5- to 15-membered)heteroarylene; and
R₁ and R₂ each independently represent hydrogen, a substituted or unsubstituted (C1-C6)alkyl, or a substituted or unsubstituted (C6-C12)aryl.

5. The organic electroluminescent compound according to claim 1, wherein Ar₁ to Ar₃ each independently represent a (C6-C30)aryl unsubstituted or substituted with at least one of (C1-C6)alkyl and (C6-C12)aryl, or a (C6-C12)aryl-substituted or unsubstituted (5- to 20-membered)heteroaryl;
L₁ and L₂ each independently represent a single bond or unsubstituted (C6-C18)arylene; and
R₁ and R₂ each independently represent hydrogen or unsubstituted (C6-C12)aryl.

6. The organic electroluminescent compound according to claim 1, wherein the compound represented by formula 1 is selected from the group consisting of:

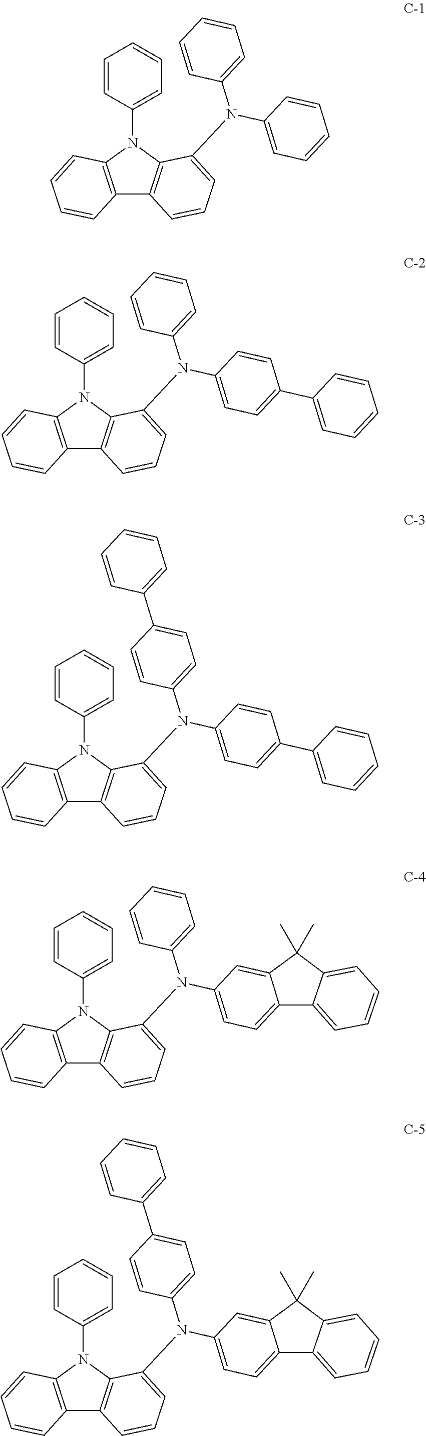 [SMILES: c1ccc(N(c2ccccc2)c2cccc3c4ccccc4n(-c4ccccc4)c23)cc1 ; c1ccc(-c2ccc(N(c3ccccc3)c3cccc4c5ccccc5n(-c5ccccc5)c34)cc2)cc1 ; c1ccc(-c2ccc(N(c3ccc(-c4ccccc4)cc3)c3cccc4c5ccccc5n(-c5ccccc5)c34)cc2)cc1 ; CC1(C)c2ccccc2-c2ccc(N(c3ccccc3)c3cccc4c5ccccc5n(-c5ccccc5)c34)cc21 ; CC1(C)c2ccccc2-c2ccc(N(c3ccc(-c4ccccc4)cc3)c3cccc4c5ccccc5n(-c5ccccc5)c34)cc21]

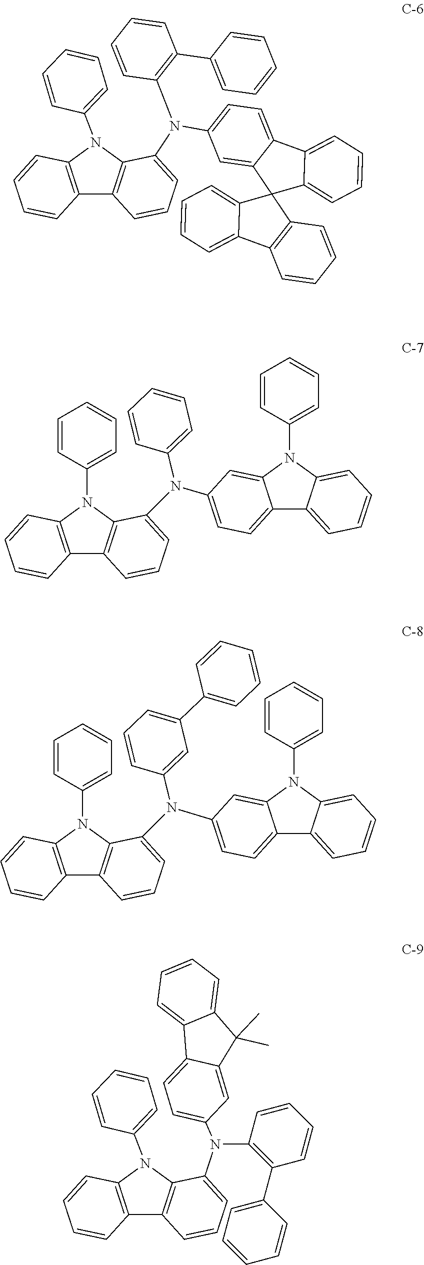 [SMILES: c1ccc(-c2ccccc2N(c2ccc3c(c2)C2(c4ccccc4-c4ccccc42)c2ccccc2-3)c2cccc3c4ccccc4n(-c4ccccc4)c23)cc1 ; c1ccc(N(c2ccc3c4ccccc4n(-c4ccccc4)c3c2)c2cccc3c4ccccc4n(-c4ccccc4)c23)cc1 ; c1ccc(-c2cccc(N(c3ccc4c5ccccc5n(-c5ccccc5)c4c3)c3cccc4c5ccccc5n(-c5ccccc5)c34)c2)cc1 ; CC1(C)c2ccccc2-c2ccc(N(c3ccccc3-c3ccccc3)c3cccc4c5ccccc5n(-c5ccccc5)c34)cc21]

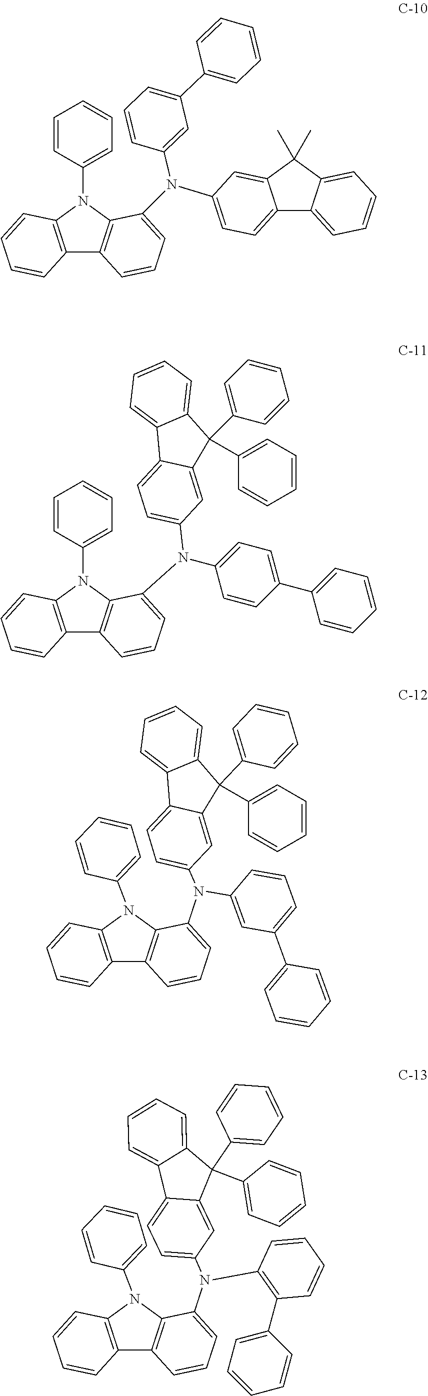 [SMILES: CC1(C)c2ccccc2-c2ccc(N(c3cccc(-c4ccccc4)c3)c3cccc4c5ccccc5n(-c5ccccc5)c34)cc21 ; c1ccc(-c2ccc(N(c3ccc4c(c3)C(c3ccccc3)(c3ccccc3)c3ccccc3-4)c3cccc4c5ccccc5n(-c5ccccc5)c34)cc2)cc1 ; c1ccc(-c2cccc(N(c3ccc4c(c3)C(c3ccccc3)(c3ccccc3)c3ccccc3-4)c3cccc4c5ccccc5n(-c5ccccc5)c34)c2)cc1 ; c1ccc(-c2ccccc2N(c2ccc3c(c2)C(c2ccccc2)(c2ccccc2)c2ccccc2-3)c2cccc3c4ccccc4n(-c4ccccc4)c23)cc1]

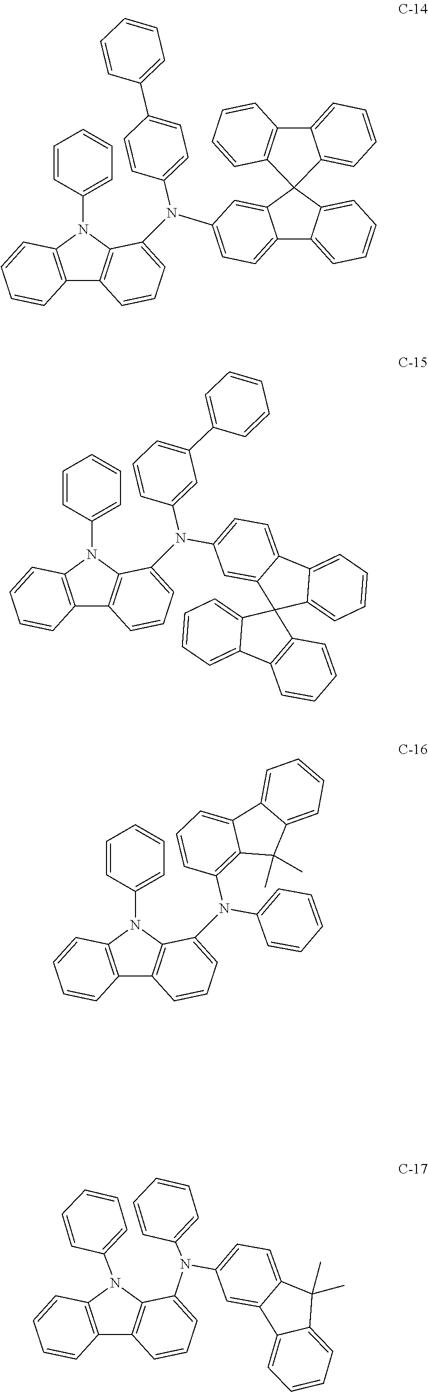 [SMILES: c1ccc(-c2ccc(N(c3ccc4c(c3)C3(c5ccccc5-c5ccccc53)c3ccccc3-4)c3cccc4c5ccccc5n(-c5ccccc5)c34)cc2)cc1 ; c1ccc(-c2cccc(N(c3ccc4c(c3)C3(c5ccccc5-c5ccccc53)c3ccccc3-4)c3cccc4c5ccccc5n(-c5ccccc5)c34)c2)cc1 ; CC1(C)c2ccccc2-c2cccc(N(c3ccccc3)c3cccc4c5ccccc5n(-c5ccccc5)c34)c21 ; CC1(C)c2ccccc2-c2cc(N(c3ccccc3)c3cccc4c5ccccc5n(-c5ccccc5)c34)ccc21]

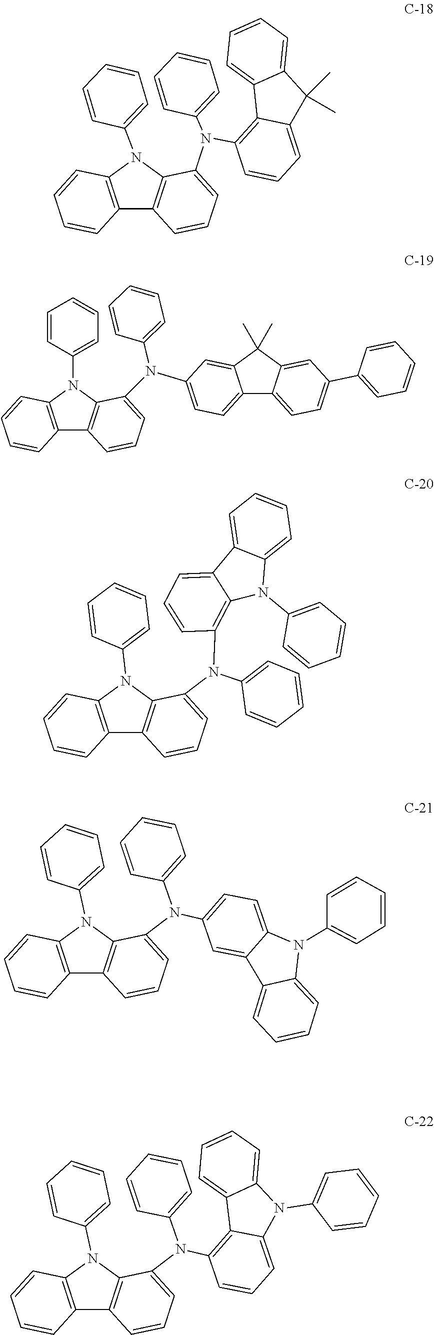 [SMILES: CC1(C)c2ccccc2-c2c(N(c3ccccc3)c3cccc4c5ccccc5n(-c5ccccc5)c34)cccc21 ; CC1(C)c2cc(-c3ccccc3)ccc2-c2ccc(N(c3ccccc3)c3cccc4c5ccccc5n(-c5ccccc5)c34)cc21 ; c1ccc(N(c2cccc3c4ccccc4n(-c4ccccc4)c23)c2cccc3c4ccccc4n(-c4ccccc4)c23)cc1 ; c1ccc(N(c2ccc3c(c2)c2ccccc2n3-c2ccccc2)c2cccc3c4ccccc4n(-c4ccccc4)c23)cc1 ; c1ccc(N(c2cccc3c2c2ccccc2n3-c2ccccc2)c2cccc3c4ccccc4n(-c4ccccc4)c23)cc1]

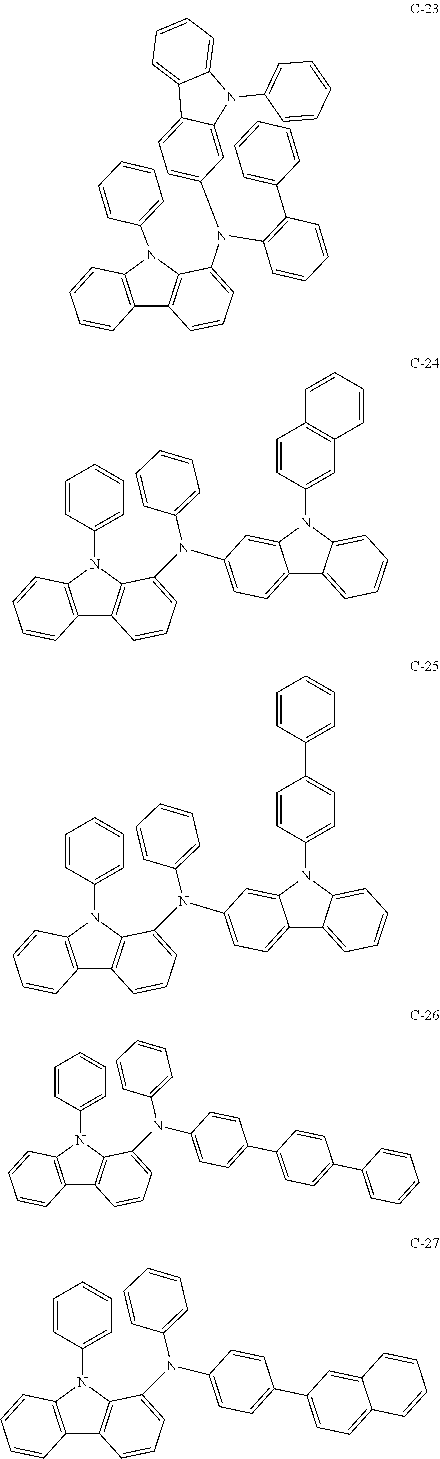 [SMILES: c1ccc(-c2ccccc2N(c2ccc3c4ccccc4n(-c4ccccc4)c3c2)c2cccc3c4ccccc4n(-c4ccccc4)c23)cc1 ; c1ccc(N(c2ccc3c4ccccc4n(-c4ccc5ccccc5c4)c3c2)c2cccc3c4ccccc4n(-c4ccccc4)c23)cc1 ; c1ccc(-c2ccc(-n3c4ccccc4c4ccc(N(c5ccccc5)c5cccc6c7ccccc7n(-c7ccccc7)c56)cc43)cc2)cc1 ; c1ccc(-c2ccc(-c3ccc(N(c4ccccc4)c4cccc5c6ccccc6n(-c6ccccc6)c45)cc3)cc2)cc1 ; c1ccc(N(c2ccc(-c3ccc4ccccc4c3)cc2)c2cccc3c4ccccc4n(-c4ccccc4)c23)cc1]

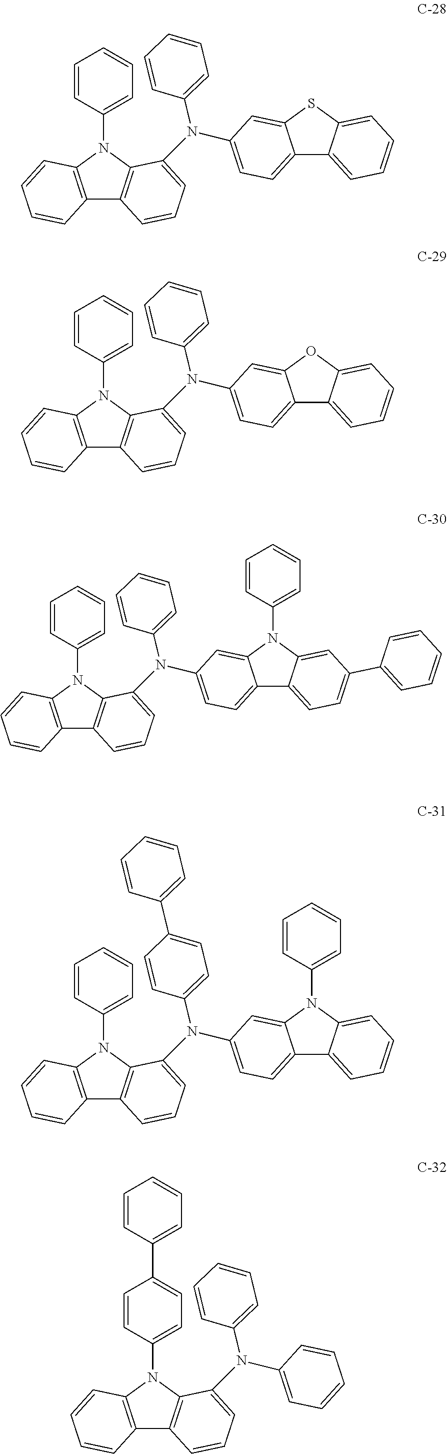 [SMILES: c1ccc(N(c2ccc3c(c2)sc2ccccc23)c2cccc3c4ccccc4n(-c4ccccc4)c23)cc1 ; c1ccc(N(c2ccc3c(c2)oc2ccccc23)c2cccc3c4ccccc4n(-c4ccccc4)c23)cc1 ; c1ccc(-c2ccc3c4ccc(N(c5ccccc5)c5cccc6c7ccccc7n(-c7ccccc7)c56)cc4n(-c4ccccc4)c3c2)cc1 ; c1ccc(-c2ccc(N(c3ccc4c5ccccc5n(-c5ccccc5)c4c3)c3cccc4c5ccccc5n(-c5ccccc5)c34)cc2)cc1 ; c1ccc(-c2ccc(-n3c4ccccc4c4cccc(N(c5ccccc5)c5ccccc5)c43)cc2)cc1]

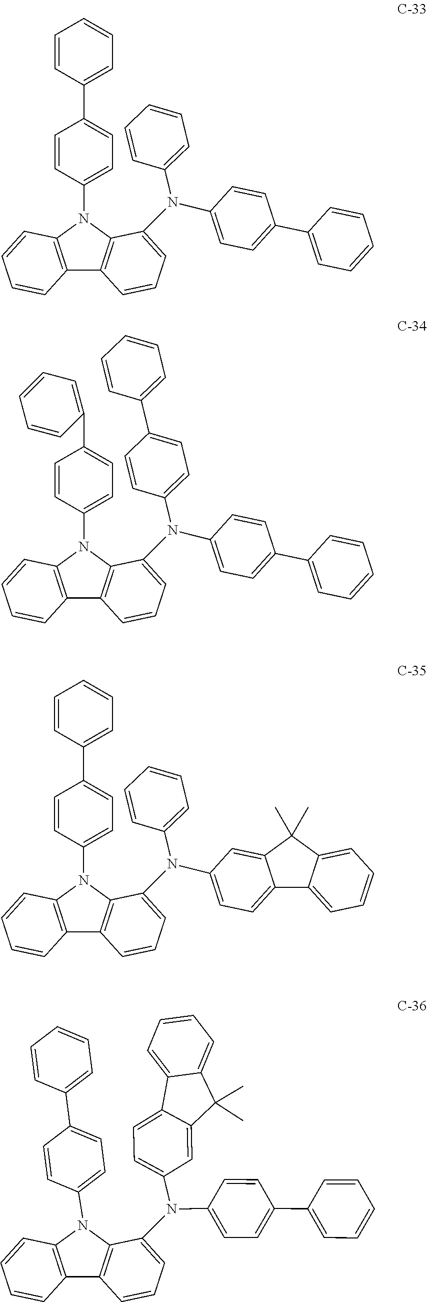

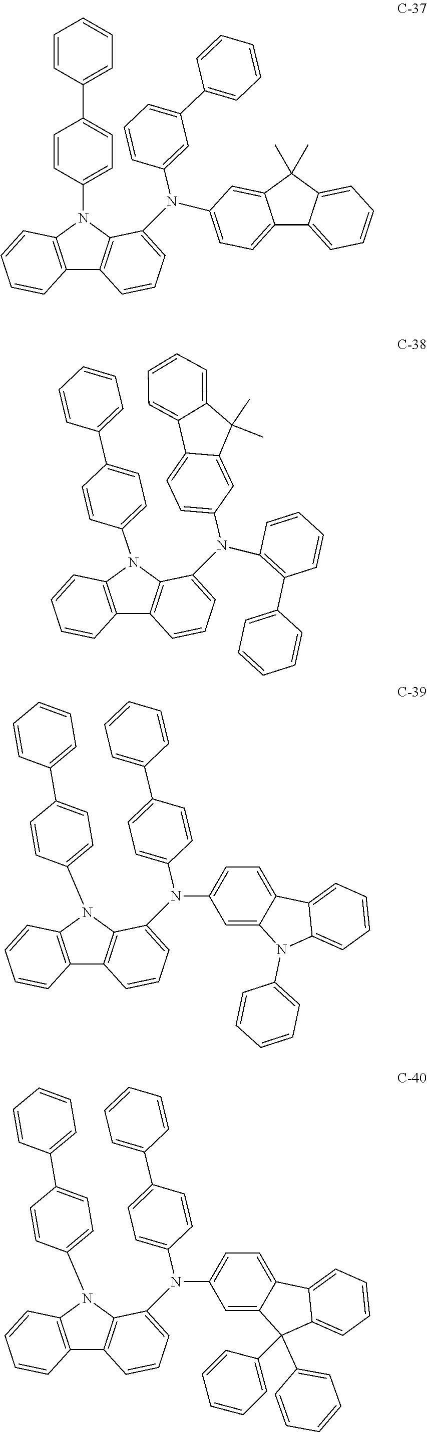 [SMILES: CC1(C)c2ccccc2-c2ccc(N(c3cccc(-c4ccccc4)c3)c3cccc4c5ccccc5n(-c5ccc(-c6ccccc6)cc5)c34)cc21 ; CC1(C)c2ccccc2-c2ccc(N(c3ccccc3-c3ccccc3)c3cccc4c5ccccc5n(-c5ccc(-c6ccccc6)cc5)c34)cc21 ; c1ccc(-c2ccc(N(c3ccc4c5ccccc5n(-c5ccccc5)c4c3)c3cccc4c5ccccc5n(-c5ccc(-c6ccccc6)cc5)c34)cc2)cc1 ; c1ccc(-c2ccc(N(c3ccc4c(c3)C(c3ccccc3)(c3ccccc3)c3ccccc3-4)c3cccc4c5ccccc5n(-c5ccc(-c6ccccc6)cc5)c34)cc2)cc1]

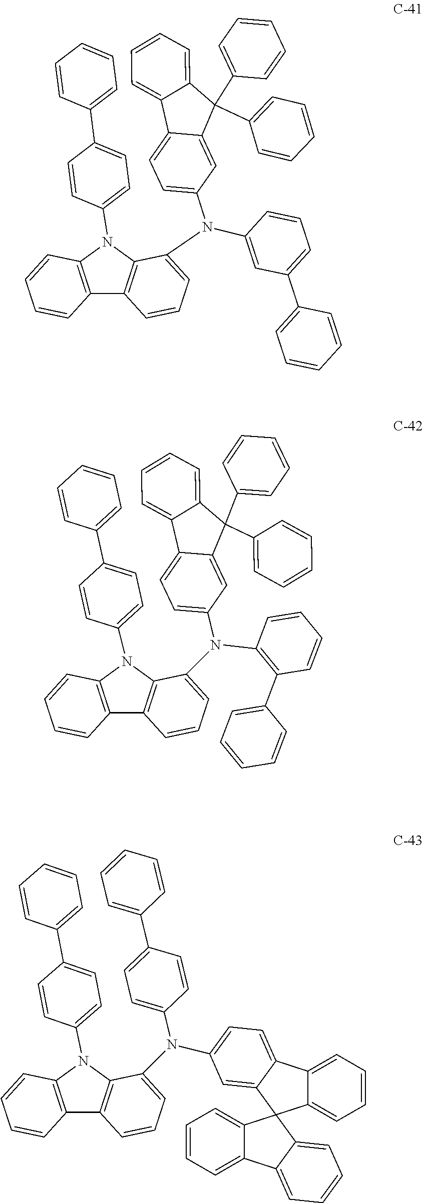 [SMILES: c1ccc(-c2ccc(-n3c4ccccc4c4cccc(N(c5cccc(-c6ccccc6)c5)c5ccc6c(c5)C(c5ccccc5)(c5ccccc5)c5ccccc5-6)c43)cc2)cc1 ; c1ccc(-c2ccc(-n3c4ccccc4c4cccc(N(c5ccc6c(c5)C(c5ccccc5)(c5ccccc5)c5ccccc5-6)c5ccccc5-c5ccccc5)c43)cc2)cc1 ; c1ccc(-c2ccc(N(c3ccc4c(c3)C3(c5ccccc5-c5ccccc53)c3ccccc3-4)c3cccc4c5ccccc5n(-c5ccc(-c6ccccc6)cc5)c34)cc2)cc1]

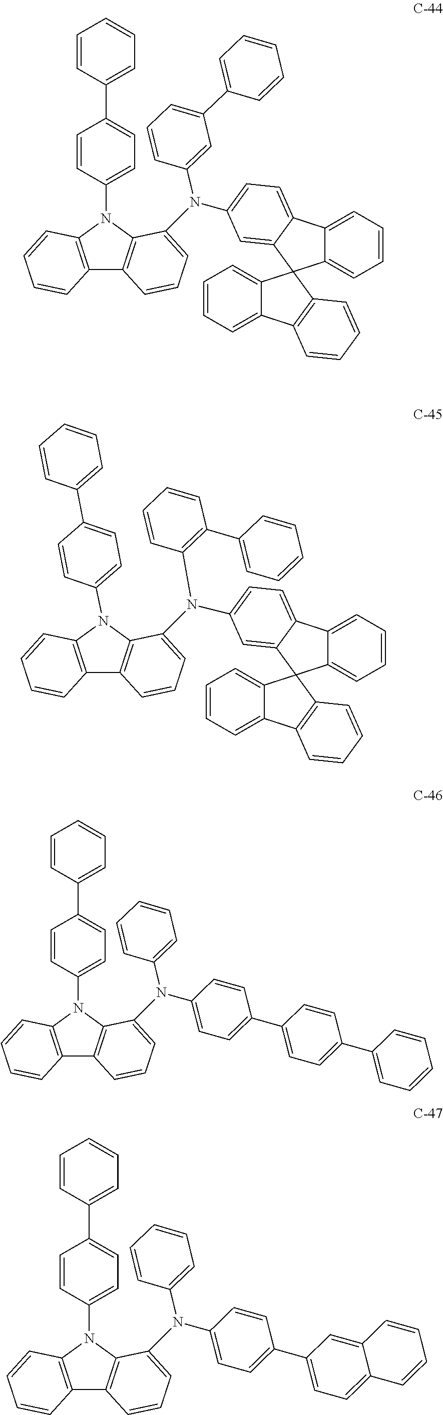 [SMILES: c1ccc(-c2ccc(-n3c4ccccc4c4cccc(N(c5cccc(-c6ccccc6)c5)c5ccc6c(c5)C5(c7ccccc7-c7ccccc75)c5ccccc5-6)c43)cc2)cc1 ; c1ccc(-c2ccc(-n3c4ccccc4c4cccc(N(c5ccc6c(c5)C5(c7ccccc7-c7ccccc75)c5ccccc5-6)c5ccccc5-c5ccccc5)c43)cc2)cc1 ; c1ccc(-c2ccc(-c3ccc(N(c4ccccc4)c4cccc5c6ccccc6n(-c6ccc(-c7ccccc7)cc6)c45)cc3)cc2)cc1 ; c1ccc(-c2ccc(-n3c4ccccc4c4cccc(N(c5ccccc5)c5ccc(-c6ccc7ccccc7c6)cc5)c43)cc2)cc1]

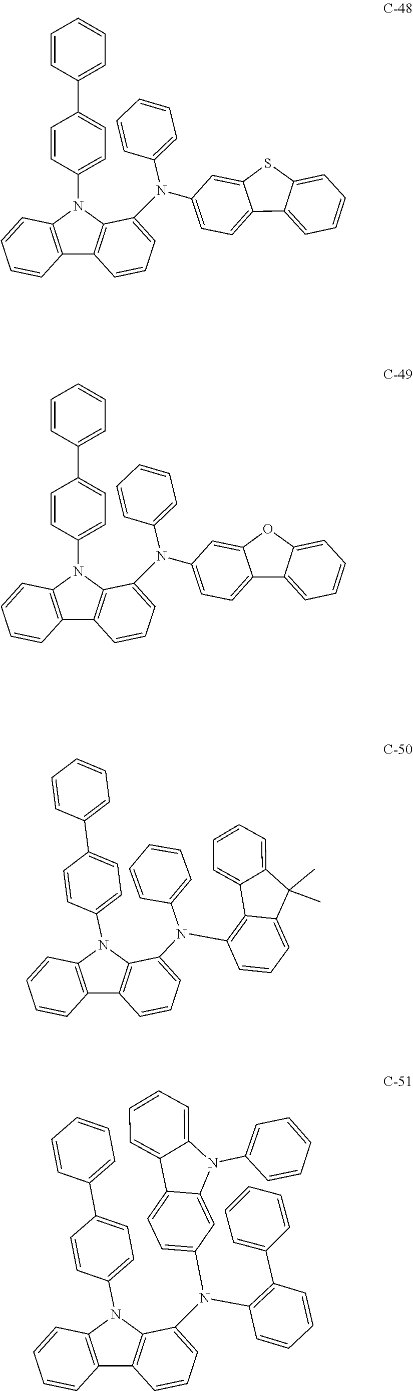 [SMILES: c1ccc(-c2ccc(-n3c4ccccc4c4cccc(N(c5ccccc5)c5ccc6c(c5)sc5ccccc56)c43)cc2)cc1 ; c1ccc(-c2ccc(-n3c4ccccc4c4cccc(N(c5ccccc5)c5ccc6c(c5)oc5ccccc56)c43)cc2)cc1 ; CC1(C)c2ccccc2-c2c(N(c3ccccc3)c3cccc4c5ccccc5n(-c5ccc(-c6ccccc6)cc5)c34)cccc21 ; c1ccc(-c2ccc(-n3c4ccccc4c4cccc(N(c5ccc6c7ccccc7n(-c7ccccc7)c6c5)c5ccccc5-c5ccccc5)c43)cc2)cc1]

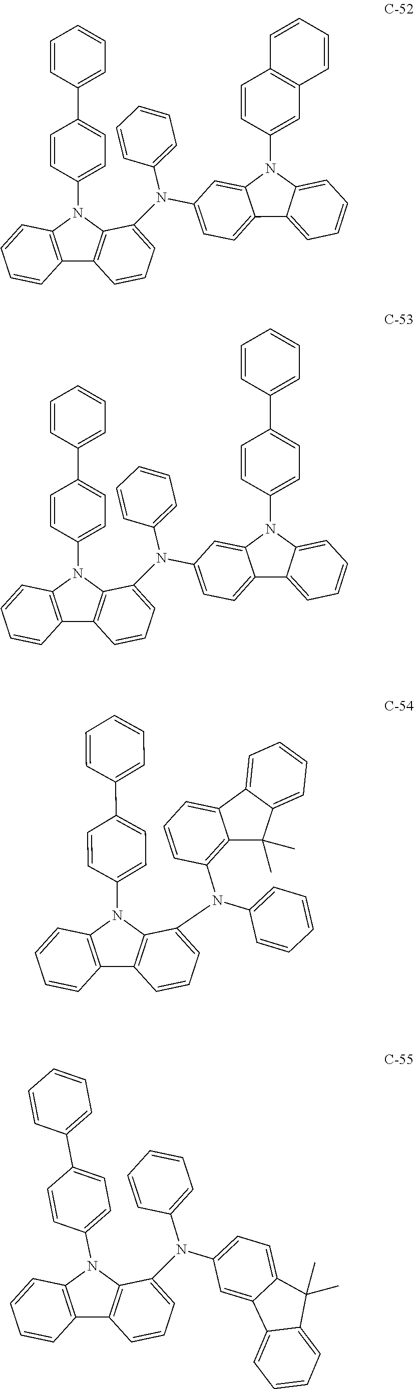 [SMILES: c1ccc(-c2ccc(-n3c4ccccc4c4cccc(N(c5ccccc5)c5ccc6c7ccccc7n(-c7ccc8ccccc8c7)c6c5)c43)cc2)cc1 ; c1ccc(-c2ccc(-n3c4ccccc4c4ccc(N(c5ccccc5)c5cccc6c7ccccc7n(-c7ccc(-c8ccccc8)cc7)c56)cc43)cc2)cc1 ; CC1(C)c2ccccc2-c2cccc(N(c3ccccc3)c3cccc4c5ccccc5n(-c5ccc(-c6ccccc6)cc5)c34)c21 ; CC1(C)c2ccccc2-c2cc(N(c3ccccc3)c3cccc4c5ccccc5n(-c5ccc(-c6ccccc6)cc5)c34)ccc21]

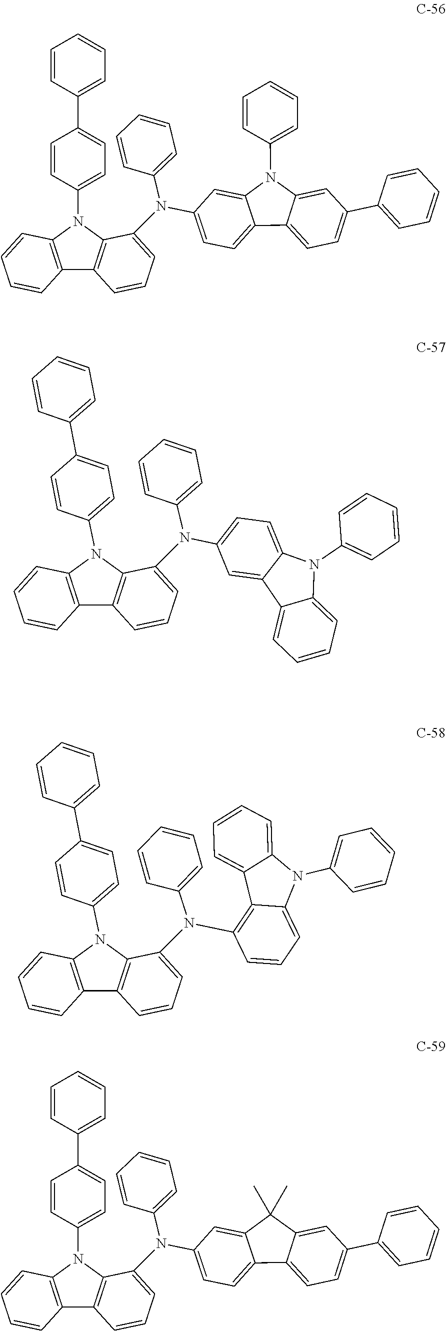 [SMILES: c1ccc(-c2ccc(-n3c4ccccc4c4cccc(N(c5ccccc5)c5ccc6c7ccc(-c8ccccc8)cc7n(-c7ccccc7)c6c5)c43)cc2)cc1 ; c1ccc(-c2ccc(-n3c4ccccc4c4cccc(N(c5ccccc5)c5ccc6c(c5)c5ccccc5n6-c5ccccc5)c43)cc2)cc1 ; c1ccc(-c2ccc(-n3c4ccccc4c4cccc(N(c5ccccc5)c5cccc6c5c5ccccc5n6-c5ccccc5)c43)cc2)cc1 ; CC1(C)c2cc(-c3ccccc3)ccc2-c2ccc(N(c3ccccc3)c3cccc4c5ccccc5n(-c5ccc(-c6ccccc6)cc5)c34)cc21]

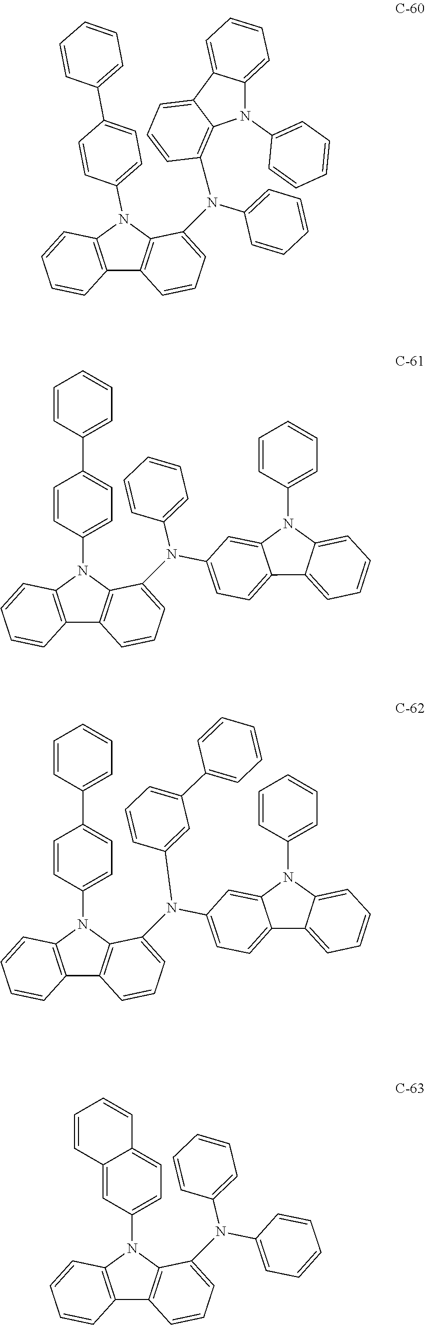 [SMILES: c1ccc(-c2ccc(-n3c4ccccc4c4cccc(N(c5ccccc5)c5cccc6c7ccccc7n(-c7ccccc7)c56)c43)cc2)cc1 ; c1ccc(-c2ccc(-n3c4ccccc4c4cccc(N(c5ccccc5)c5ccc6c7ccccc7n(-c7ccccc7)c6c5)c43)cc2)cc1 ; c1ccc(-c2ccc(-n3c4ccccc4c4cccc(N(c5cccc(-c6ccccc6)c5)c5ccc6c7ccccc7n(-c7ccccc7)c6c5)c43)cc2)cc1 ; c1ccc(N(c2ccccc2)c2cccc3c4ccccc4n(-c4ccc5ccccc5c4)c23)cc1]

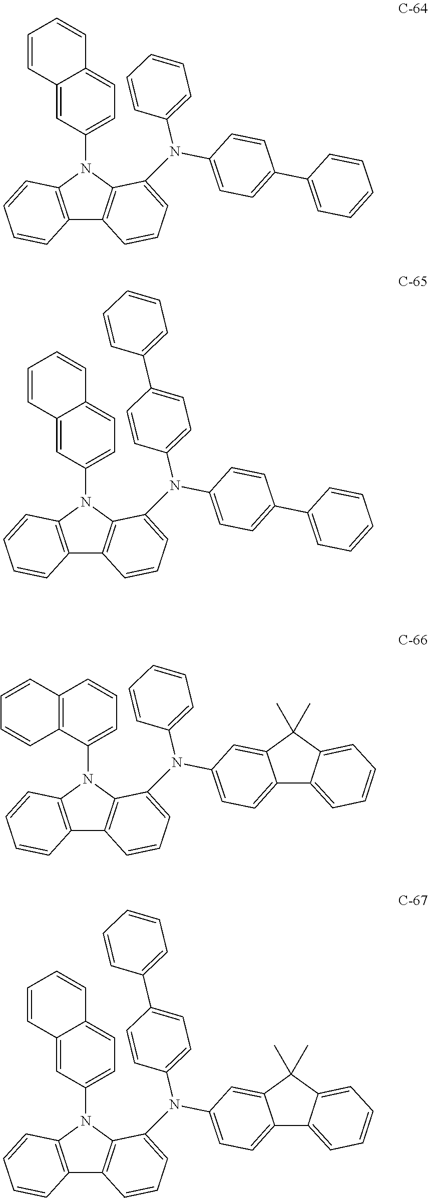 [SMILES: c1ccc(-c2ccc(N(c3ccccc3)c3cccc4c5ccccc5n(-c5ccc6ccccc6c5)c34)cc2)cc1 ; c1ccc(-c2ccc(N(c3ccc(-c4ccccc4)cc3)c3cccc4c5ccccc5n(-c5ccc6ccccc6c5)c34)cc2)cc1 ; CC1(C)c2ccccc2-c2ccc(N(c3ccccc3)c3cccc4c5ccccc5n(-c5cccc6ccccc56)c34)cc21 ; CC1(C)c2ccccc2-c2ccc(N(c3ccc(-c4ccccc4)cc3)c3cccc4c5ccccc5n(-c5ccc6ccccc6c5)c34)cc21]

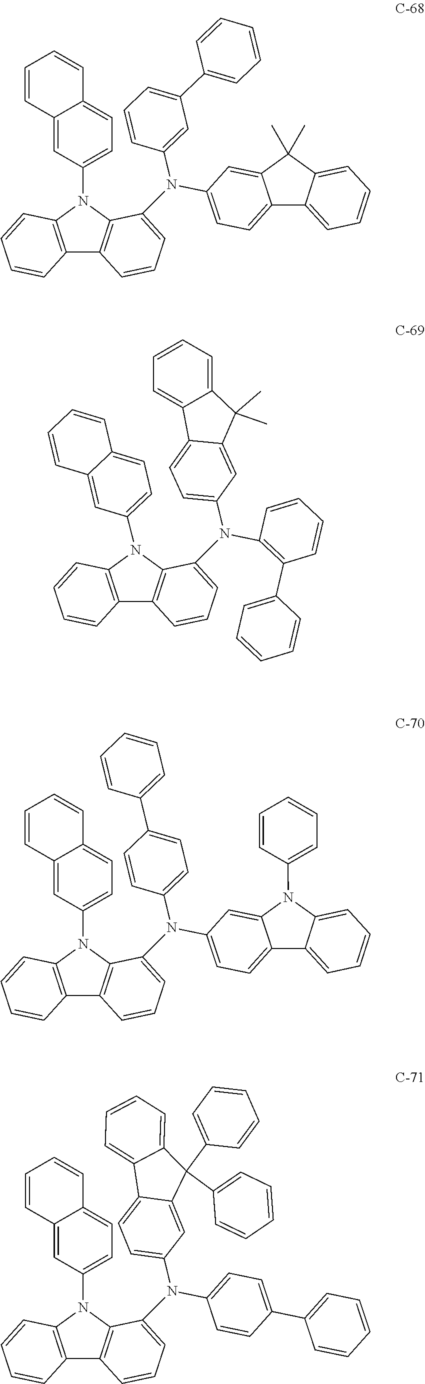 [SMILES: CC1(C)c2ccccc2-c2ccc(N(c3cccc(-c4ccccc4)c3)c3cccc4c5ccccc5n(-c5ccc6ccccc6c5)c34)cc21 ; CC1(C)c2ccccc2-c2ccc(N(c3ccccc3-c3ccccc3)c3cccc4c5ccccc5n(-c5ccc6ccccc6c5)c34)cc21 ; c1ccc(-c2ccc(N(c3ccc4c5ccccc5n(-c5ccccc5)c4c3)c3cccc4c5ccccc5n(-c5ccc6ccccc6c5)c34)cc2)cc1 ; c1ccc(-c2ccc(N(c3ccc4c(c3)C(c3ccccc3)(c3ccccc3)c3ccccc3-4)c3cccc4c5ccccc5n(-c5ccc6ccccc6c5)c34)cc2)cc1]

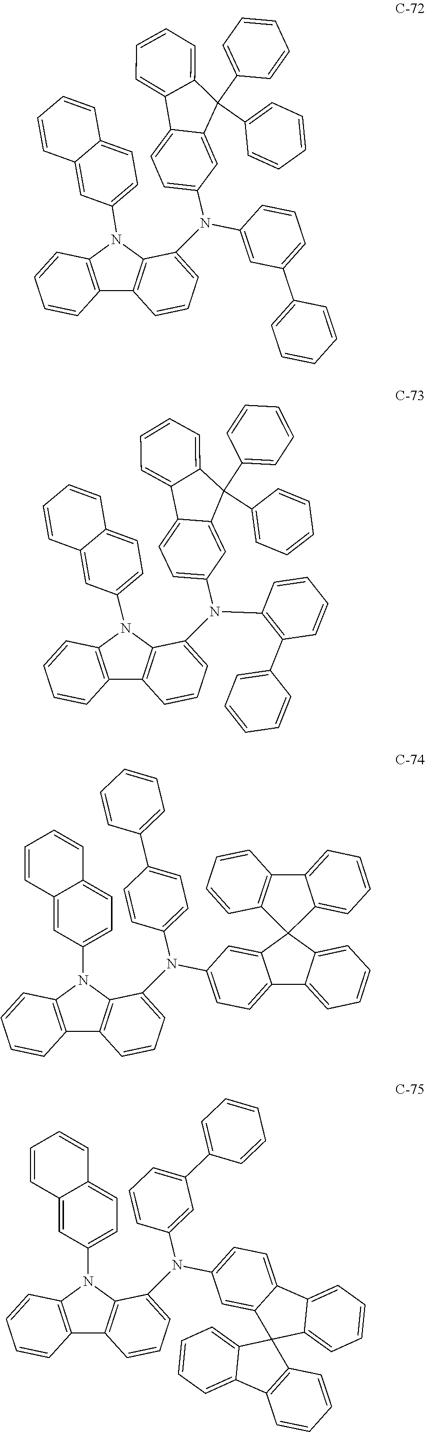 [SMILES: c1ccc(-c2cccc(N(c3ccc4c(c3)C(c3ccccc3)(c3ccccc3)c3ccccc3-4)c3cccc4c5ccccc5n(-c5ccc6ccccc6c5)c34)c2)cc1 ; c1ccc(-c2ccccc2N(c2ccc3c(c2)C(c2ccccc2)(c2ccccc2)c2ccccc2-3)c2cccc3c4ccccc4n(-c4ccc5ccccc5c4)c23)cc1 ; c1ccc(-c2ccc(N(c3ccc4c(c3)C3(c5ccccc5-c5ccccc53)c3ccccc3-4)c3cccc4c5ccccc5n(-c5ccc6ccccc6c5)c34)cc2)cc1 ; c1ccc(-c2cccc(N(c3ccc4c(c3)C3(c5ccccc5-c5ccccc53)c3ccccc3-4)c3cccc4c5ccccc5n(-c5ccc6ccccc6c5)c34)c2)cc1]

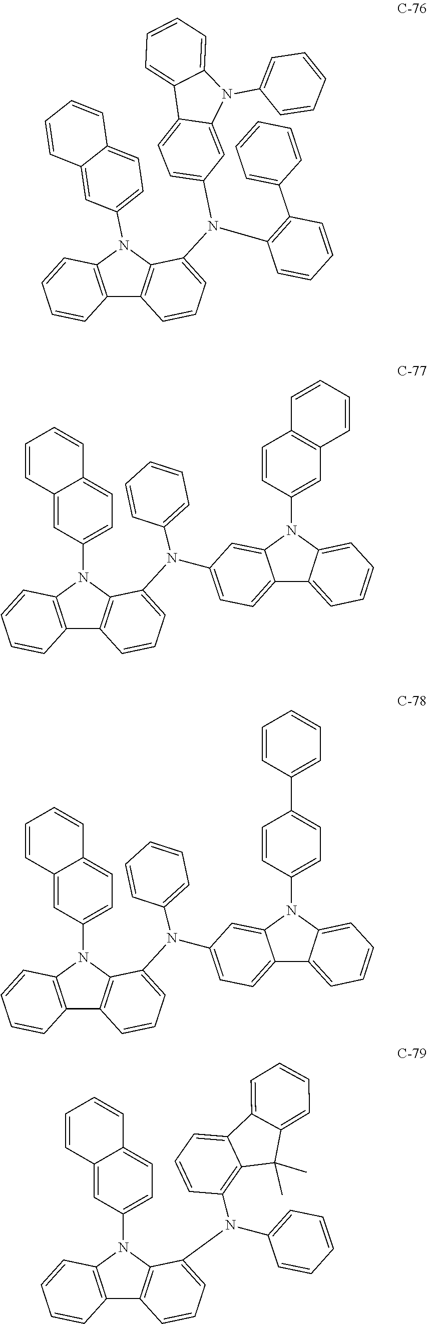 [SMILES: c1ccc(-c2ccccc2N(c2ccc3c4ccccc4n(-c4ccccc4)c3c2)c2cccc3c4ccccc4n(-c4ccc5ccccc5c4)c23)cc1 ; c1ccc(N(c2ccc3c4ccccc4n(-c4ccc5ccccc5c4)c3c2)c2cccc3c4ccccc4n(-c4ccc5ccccc5c4)c23)cc1 ; c1ccc(-c2ccc(-n3c4ccccc4c4ccc(N(c5ccccc5)c5cccc6c7ccccc7n(-c7ccc8ccccc8c7)c56)cc43)cc2)cc1 ; CC1(C)c2ccccc2-c2cccc(N(c3ccccc3)c3cccc4c5ccccc5n(-c5ccc6ccccc6c5)c34)c21]

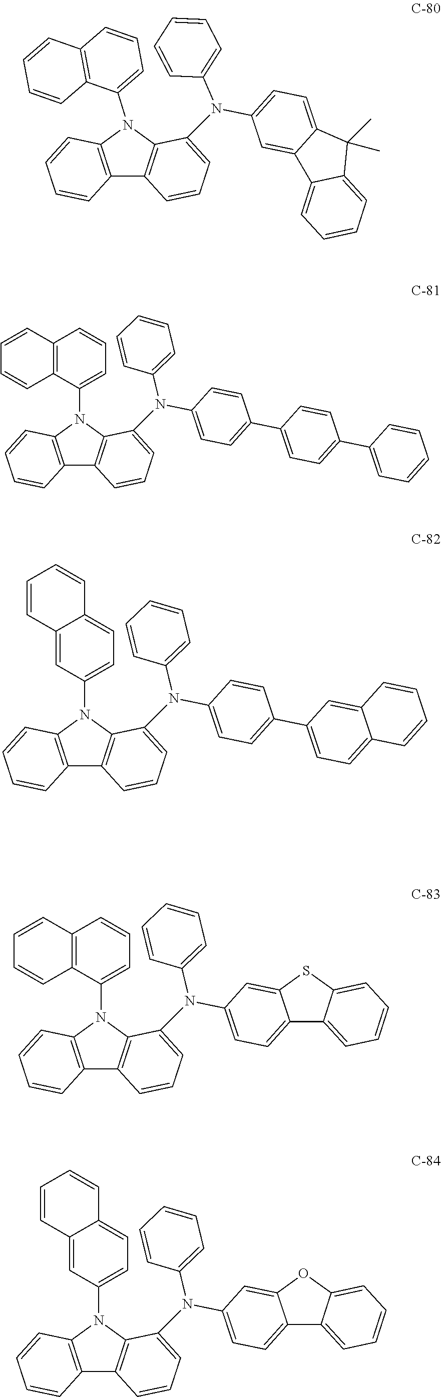 [SMILES: CC1(C)c2ccccc2-c2cc(N(c3ccccc3)c3cccc4c5ccccc5n(-c5cccc6ccccc56)c34)ccc21 ; c1ccc(-c2ccc(-c3ccc(N(c4ccccc4)c4cccc5c6ccccc6n(-c6cccc7ccccc67)c45)cc3)cc2)cc1 ; c1ccc(N(c2ccc(-c3ccc4ccccc4c3)cc2)c2cccc3c4ccccc4n(-c4ccc5ccccc5c4)c23)cc1 ; c1ccc(N(c2ccc3c(c2)sc2ccccc23)c2cccc3c4ccccc4n(-c4cccc5ccccc45)c23)cc1 ; c1ccc(N(c2ccc3c(c2)oc2ccccc23)c2cccc3c4ccccc4n(-c4ccc5ccccc5c4)c23)cc1]

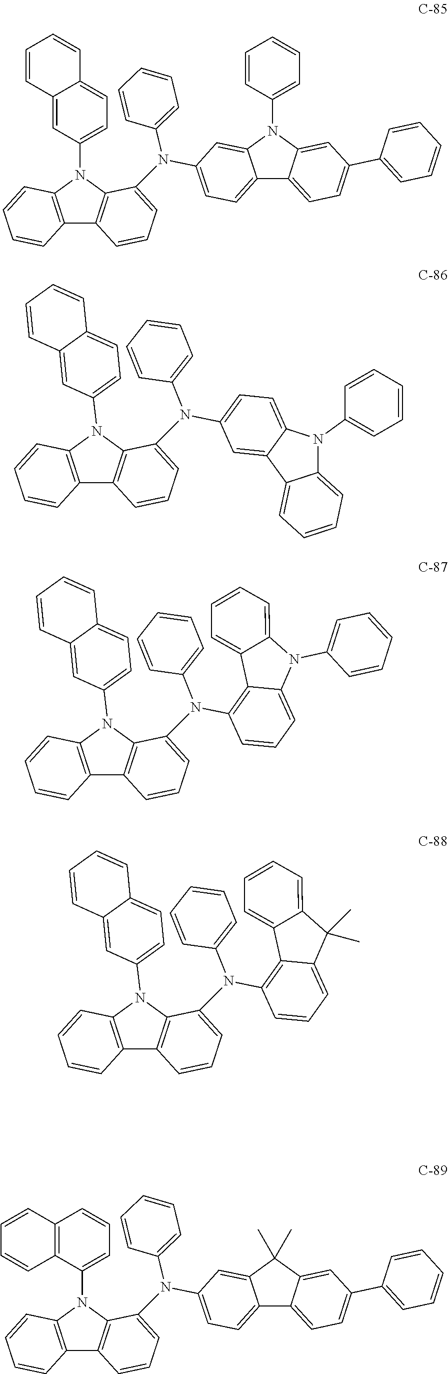

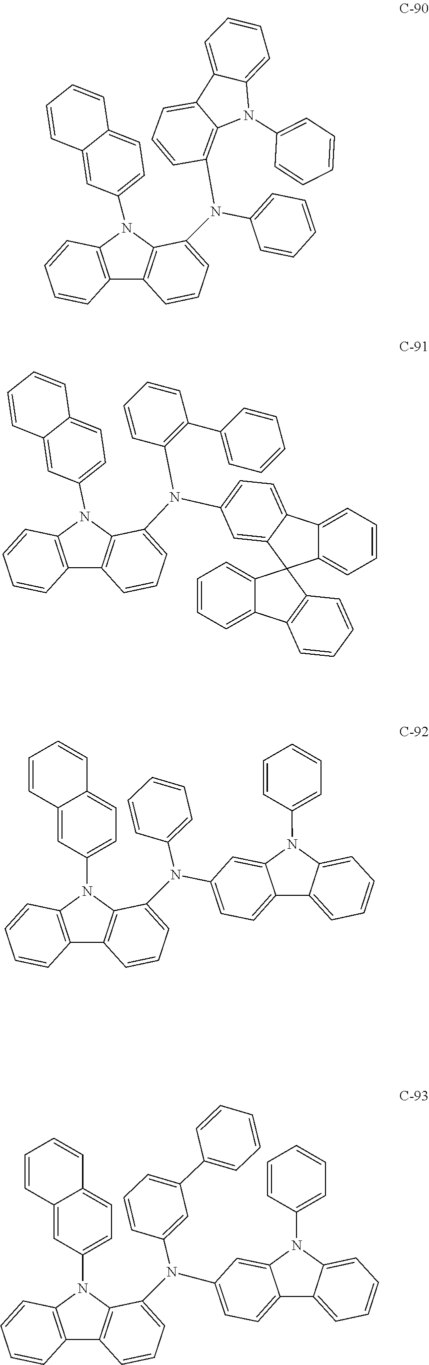 [SMILES: c1ccc(N(c2cccc3c4ccccc4n(-c4ccccc4)c23)c2cccc3c4ccccc4n(-c4ccc5ccccc5c4)c23)cc1 ; c1ccc(-c2ccccc2N(c2ccc3c(c2)C2(c4ccccc4-c4ccccc42)c2ccccc2-3)c2cccc3c4ccccc4n(-c4ccc5ccccc5c4)c23)cc1 ; c1ccc(N(c2ccc3c4ccccc4n(-c4ccccc4)c3c2)c2cccc3c4ccccc4n(-c4ccc5ccccc5c4)c23)cc1 ; c1ccc(-c2cccc(N(c3ccc4c5ccccc5n(-c5ccccc5)c4c3)c3cccc4c5ccccc5n(-c5ccc6ccccc6c5)c34)c2)cc1]

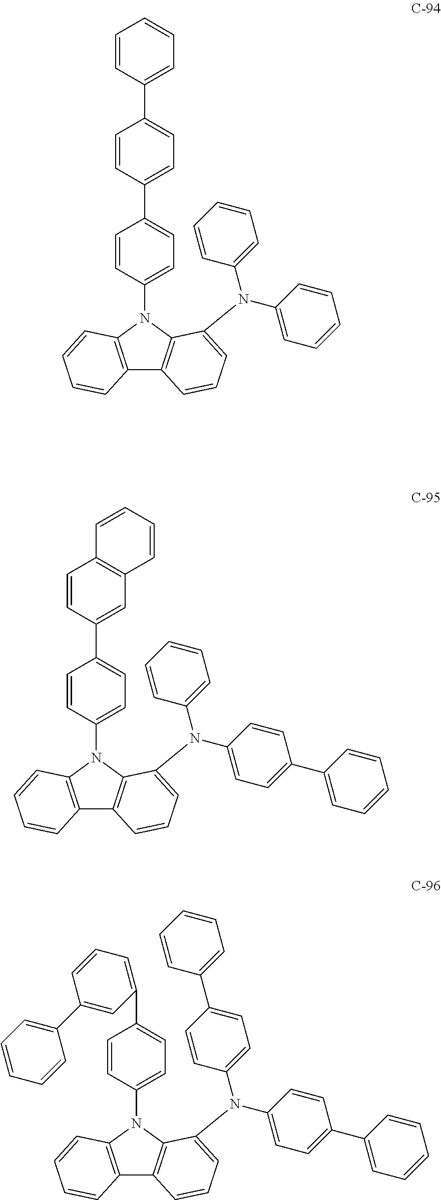 [SMILES: c1ccc(-c2ccc(-c3ccc(-n4c5ccccc5c5cccc(N(c6ccccc6)c6ccccc6)c54)cc3)cc2)cc1 ; c1ccc(-c2ccc(N(c3ccccc3)c3cccc4c5ccccc5n(-c5ccc(-c6ccc7ccccc7c6)cc5)c34)cc2)cc1 ; c1ccc(-c2ccc(N(c3ccc(-c4ccccc4)cc3)c3cccc4c5ccccc5n(-c5ccc(-c6cccc(-c7ccccc7)c6)cc5)c34)cc2)cc1]

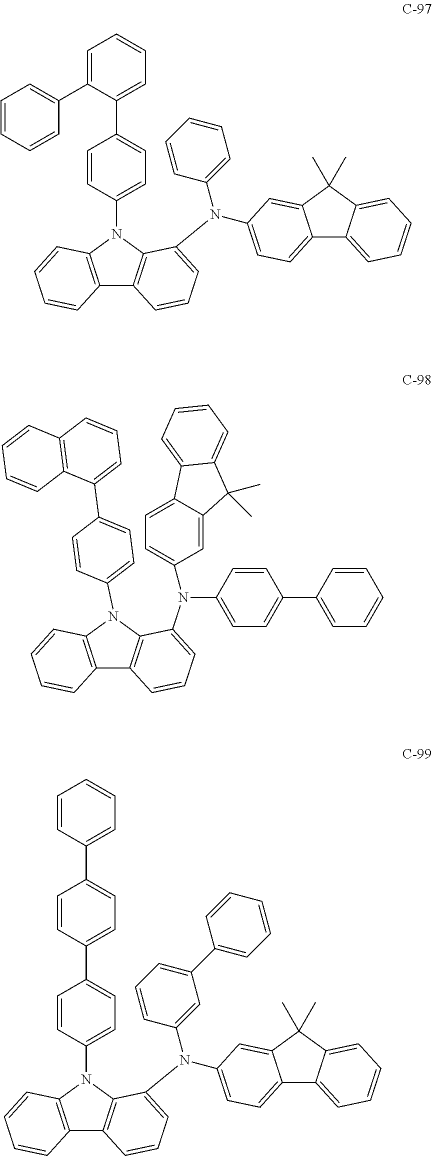 [SMILES: CC1(C)c2ccccc2-c2ccc(N(c3ccccc3)c3cccc4c5ccccc5n(-c5ccc(-c6ccccc6-c6ccccc6)cc5)c34)cc21 ; CC1(C)c2ccccc2-c2ccc(N(c3ccc(-c4ccccc4)cc3)c3cccc4c5ccccc5n(-c5ccc(-c6cccc7ccccc67)cc5)c34)cc21 ; CC1(C)c2ccccc2-c2ccc(N(c3cccc(-c4ccccc4)c3)c3cccc4c5ccccc5n(-c5ccc(-c6ccc(-c7ccccc7)cc6)cc5)c34)cc21]

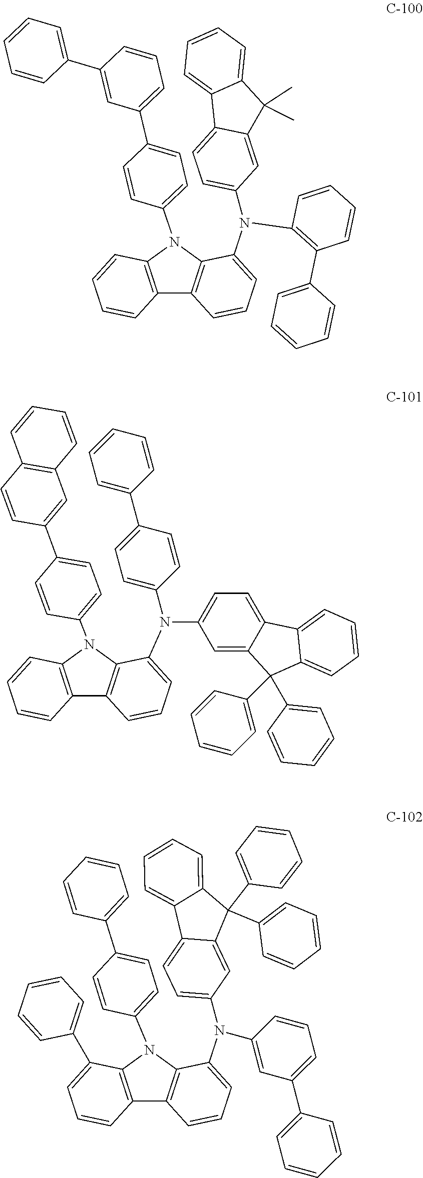 [SMILES: CC1(C)c2ccccc2-c2ccc(N(c3ccccc3-c3ccccc3)c3cccc4c5ccccc5n(-c5ccc(-c6cccc(-c7ccccc7)c6)cc5)c34)cc21 ; c1ccc(-c2ccc(N(c3ccc4c(c3)C(c3ccccc3)(c3ccccc3)c3ccccc3-4)c3cccc4c5ccccc5n(-c5ccc(-c6ccc7ccccc7c6)cc5)c34)cc2)cc1 ; c1ccc(-c2ccc(-n3c4c(-c5ccccc5)cccc4c4cccc(N(c5cccc(-c6ccccc6)c5)c5ccc6c(c5)C(c5ccccc5)(c5ccccc5)c5ccccc5-6)c43)cc2)cc1]

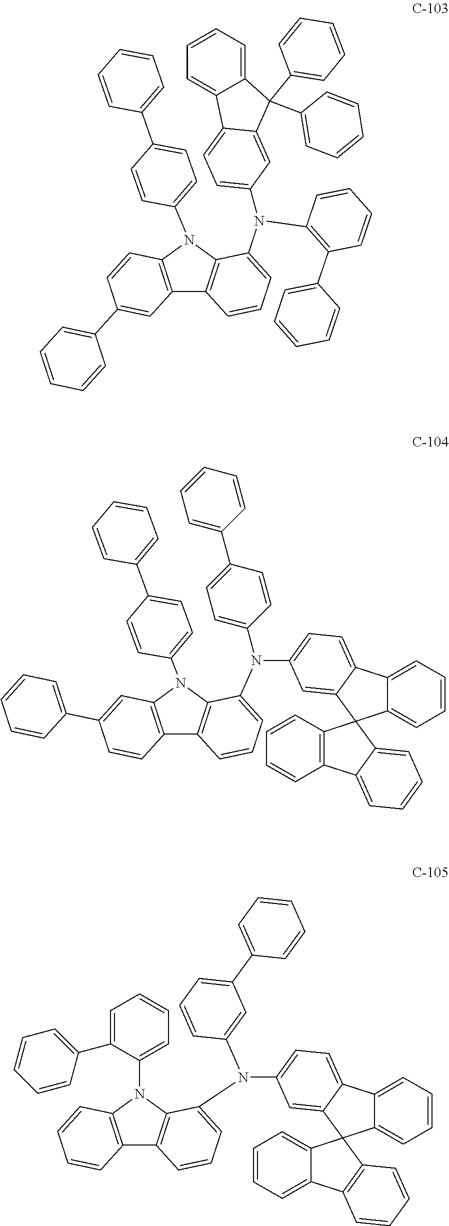 [SMILES: c1ccc(-c2ccc(-n3c4ccc(-c5ccccc5)cc4c4cccc(N(c5ccc6c(c5)C(c5ccccc5)(c5ccccc5)c5ccccc5-6)c5ccccc5-c5ccccc5)c43)cc2)cc1 ; c1ccc(-c2ccc(N(c3ccc4c(c3)C3(c5ccccc5-c5ccccc53)c3ccccc3-4)c3cccc4c5ccc(-c6ccccc6)cc5n(-c5ccc(-c6ccccc6)cc5)c34)cc2)cc1 ; c1ccc(-c2cccc(N(c3ccc4c(c3)C3(c5ccccc5-c5ccccc53)c3ccccc3-4)c3cccc4c5ccccc5n(-c5ccccc5-c5ccccc5)c34)c2)cc1]

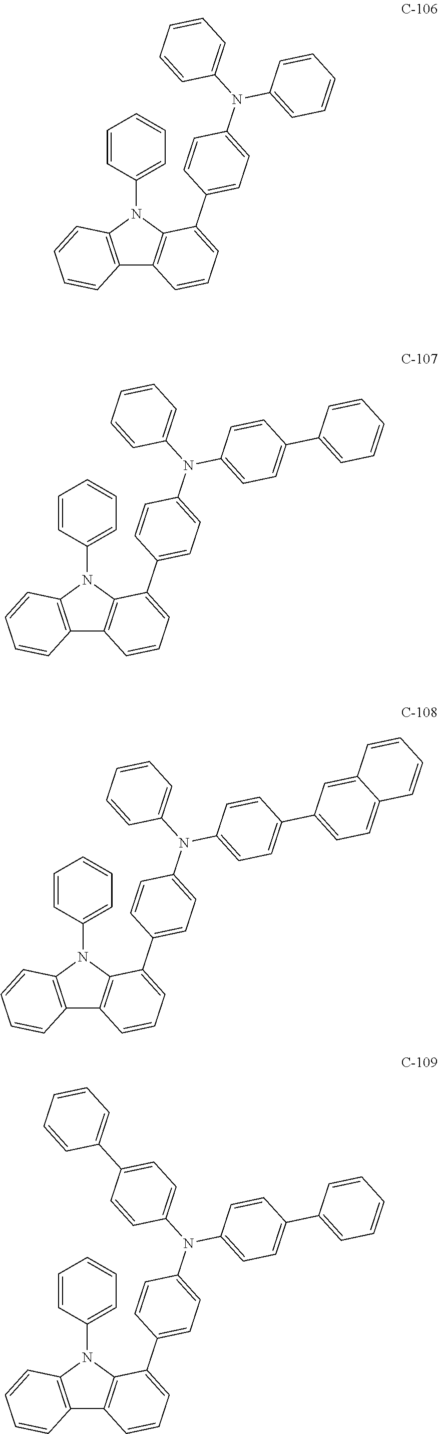 [SMILES: c1ccc(N(c2ccccc2)c2ccc(-c3cccc4c5ccccc5n(-c5ccccc5)c34)cc2)cc1 ; c1ccc(-c2ccc(N(c3ccccc3)c3ccc(-c4cccc5c6ccccc6n(-c6ccccc6)c45)cc3)cc2)cc1 ; c1ccc(N(c2ccc(-c3ccc4ccccc4c3)cc2)c2ccc(-c3cccc4c5ccccc5n(-c5ccccc5)c34)cc2)cc1 ; c1ccc(-c2ccc(N(c3ccc(-c4ccccc4)cc3)c3ccc(-c4cccc5c6ccccc6n(-c6ccccc6)c45)cc3)cc2)cc1]

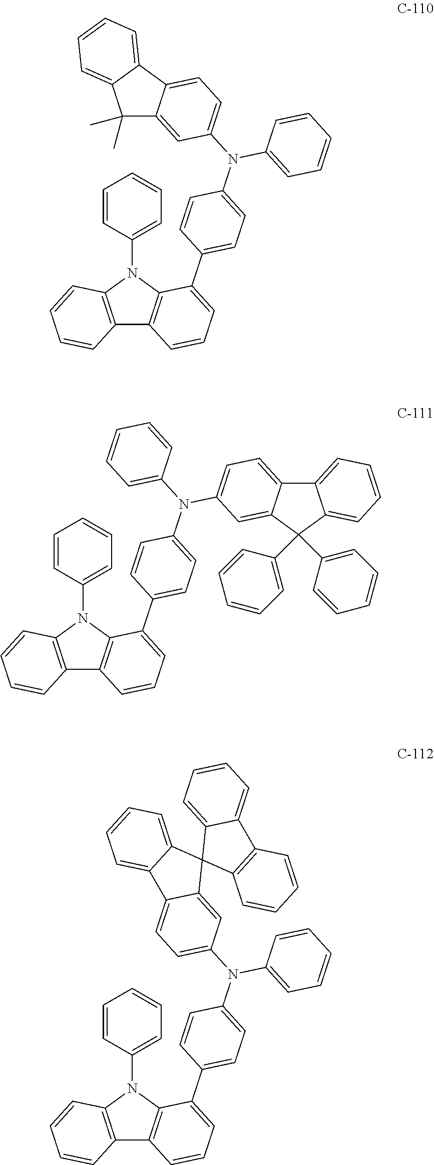 [SMILES: CC1(C)c2ccccc2-c2ccc(N(c3ccccc3)c3ccc(-c4cccc5c6ccccc6n(-c6ccccc6)c45)cc3)cc21 ; c1ccc(N(c2ccc(-c3cccc4c5ccccc5n(-c5ccccc5)c34)cc2)c2ccc3c(c2)C(c2ccccc2)(c2ccccc2)c2ccccc2-3)cc1 ; c1ccc(N(c2ccc(-c3cccc4c5ccccc5n(-c5ccccc5)c34)cc2)c2ccc3c(c2)C2(c4ccccc4-c4ccccc42)c2ccccc2-3)cc1]

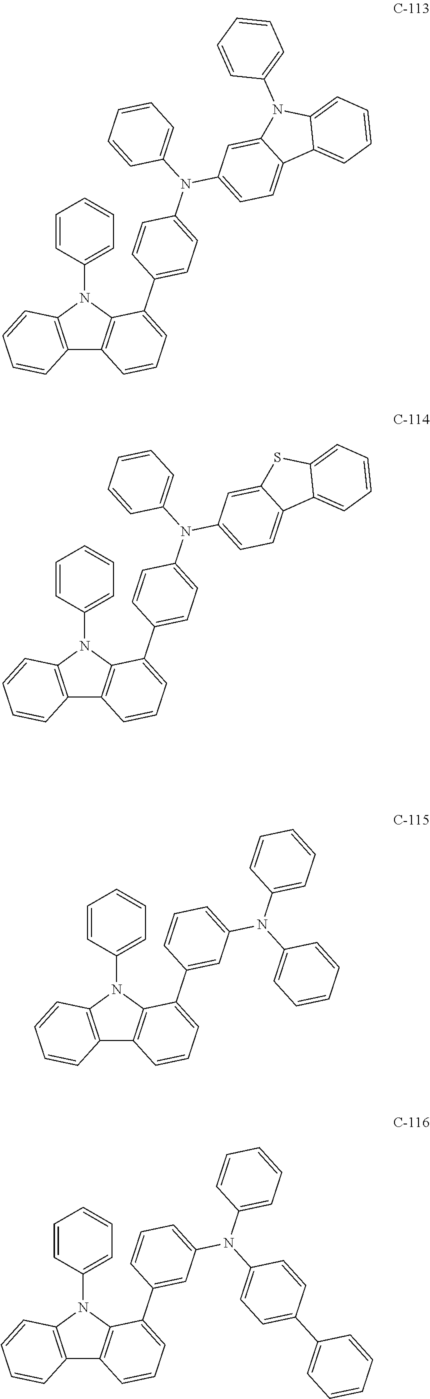 [SMILES: c1ccc(N(c2ccc(-c3cccc4c5ccccc5n(-c5ccccc5)c34)cc2)c2ccc3c4ccccc4n(-c4ccccc4)c3c2)cc1 ; c1ccc(N(c2ccc(-c3cccc4c5ccccc5n(-c5ccccc5)c34)cc2)c2ccc3c(c2)sc2ccccc23)cc1 ; c1ccc(N(c2ccccc2)c2cccc(-c3cccc4c5ccccc5n(-c5ccccc5)c34)c2)cc1 ; c1ccc(-c2ccc(N(c3ccccc3)c3cccc(-c4cccc5c6ccccc6n(-c6ccccc6)c45)c3)cc2)cc1]

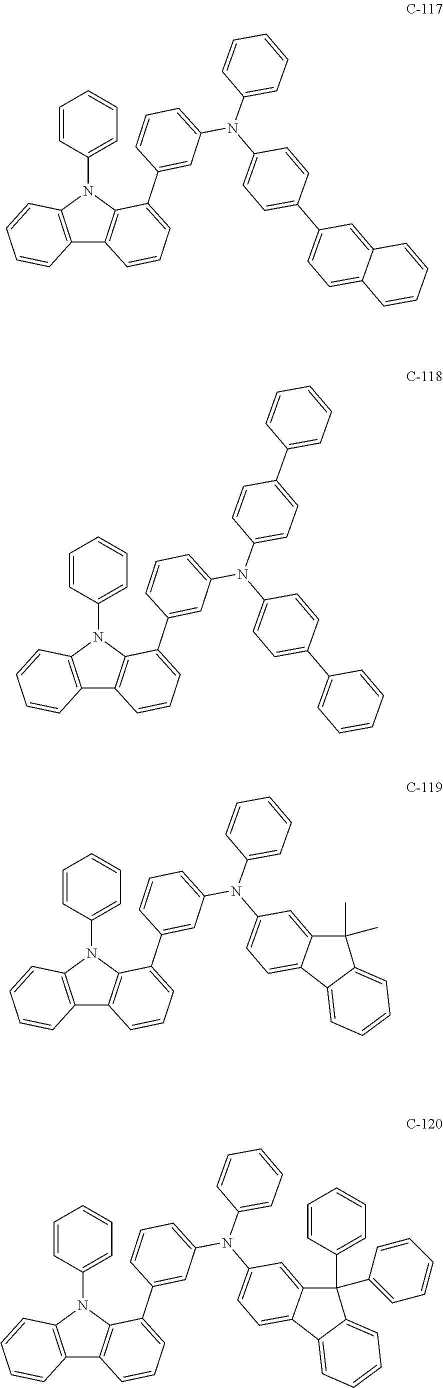 [SMILES: c1ccc(N(c2ccc(-c3ccc4ccccc4c3)cc2)c2cccc(-c3cccc4c5ccccc5n(-c5ccccc5)c34)c2)cc1 ; c1ccc(-c2ccc(N(c3ccc(-c4ccccc4)cc3)c3cccc(-c4cccc5c6ccccc6n(-c6ccccc6)c45)c3)cc2)cc1 ; CC1(C)c2ccccc2-c2ccc(N(c3ccccc3)c3cccc(-c4cccc5c6ccccc6n(-c6ccccc6)c45)c3)cc21 ; c1ccc(N(c2cccc(-c3cccc4c5ccccc5n(-c5ccccc5)c34)c2)c2ccc3c(c2)C(c2ccccc2)(c2ccccc2)c2ccccc2-3)cc1]

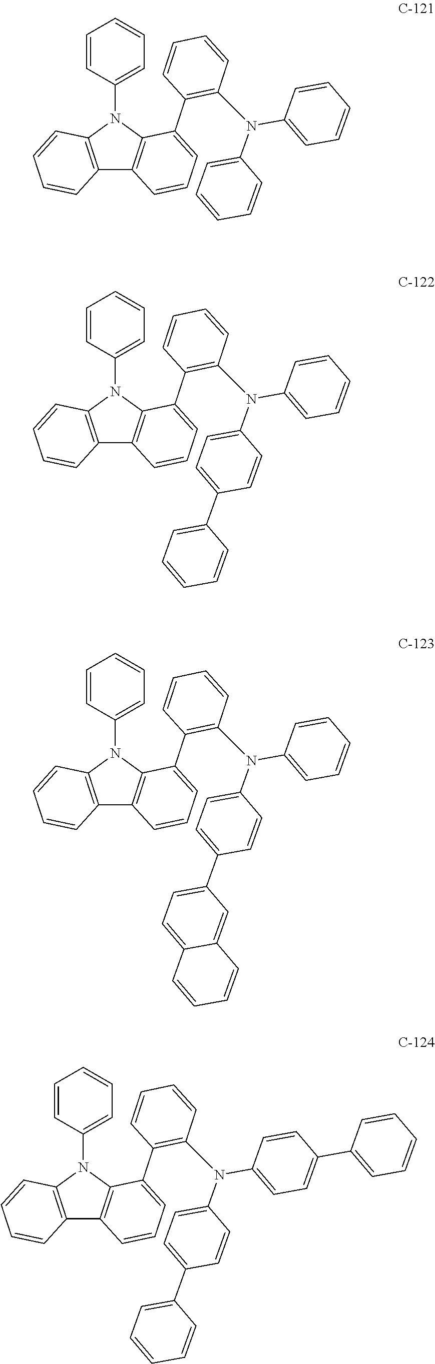 [SMILES: c1ccc(N(c2ccccc2)c2ccccc2-c2cccc3c4ccccc4n(-c4ccccc4)c23)cc1 ; c1ccc(-c2ccc(N(c3ccccc3)c3ccccc3-c3cccc4c5ccccc5n(-c5ccccc5)c34)cc2)cc1 ; c1ccc(N(c2ccc(-c3ccc4ccccc4c3)cc2)c2ccccc2-c2cccc3c4ccccc4n(-c4ccccc4)c23)cc1 ; c1ccc(-c2ccc(N(c3ccc(-c4ccccc4)cc3)c3ccccc3-c3cccc4c5ccccc5n(-c5ccccc5)c34)cc2)cc1]

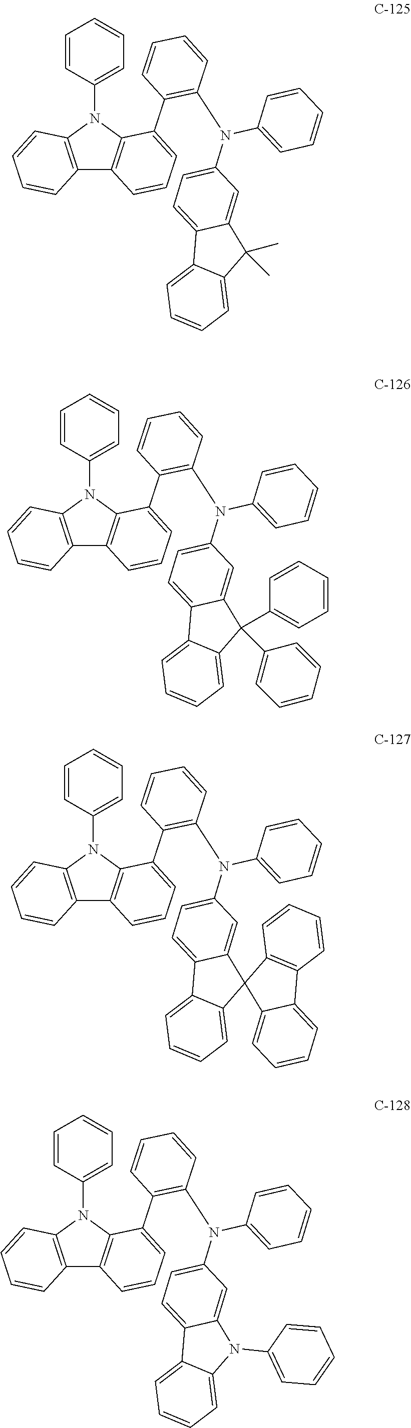 [SMILES: CC1(C)c2ccccc2-c2ccc(N(c3ccccc3)c3ccccc3-c3cccc4c5ccccc5n(-c5ccccc5)c34)cc21 ; c1ccc(N(c2ccc3c(c2)C(c2ccccc2)(c2ccccc2)c2ccccc2-3)c2ccccc2-c2cccc3c4ccccc4n(-c4ccccc4)c23)cc1 ; c1ccc(N(c2ccc3c(c2)C2(c4ccccc4-c4ccccc42)c2ccccc2-3)c2ccccc2-c2cccc3c4ccccc4n(-c4ccccc4)c23)cc1 ; c1ccc(N(c2ccc3c4ccccc4n(-c4ccccc4)c3c2)c2ccccc2-c2cccc3c4ccccc4n(-c4ccccc4)c23)cc1]

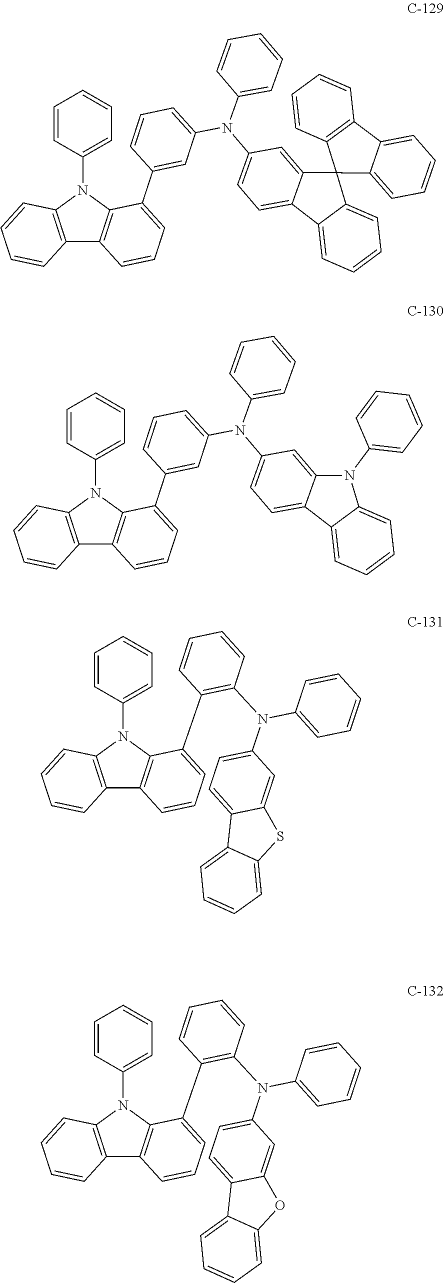 [SMILES: c1ccc(N(c2cccc(-c3cccc4c5ccccc5n(-c5ccccc5)c34)c2)c2ccc3c(c2)C2(c4ccccc4-c4ccccc42)c2ccccc2-3)cc1 ; c1ccc(N(c2cccc(-c3cccc4c5ccccc5n(-c5ccccc5)c34)c2)c2ccc3c4ccccc4n(-c4ccccc4)c3c2)cc1 ; c1ccc(N(c2ccc3c(c2)sc2ccccc23)c2ccccc2-c2cccc3c4ccccc4n(-c4ccccc4)c23)cc1 ; c1ccc(N(c2ccc3c(c2)oc2ccccc23)c2ccccc2-c2cccc3c4ccccc4n(-c4ccccc4)c23)cc1]

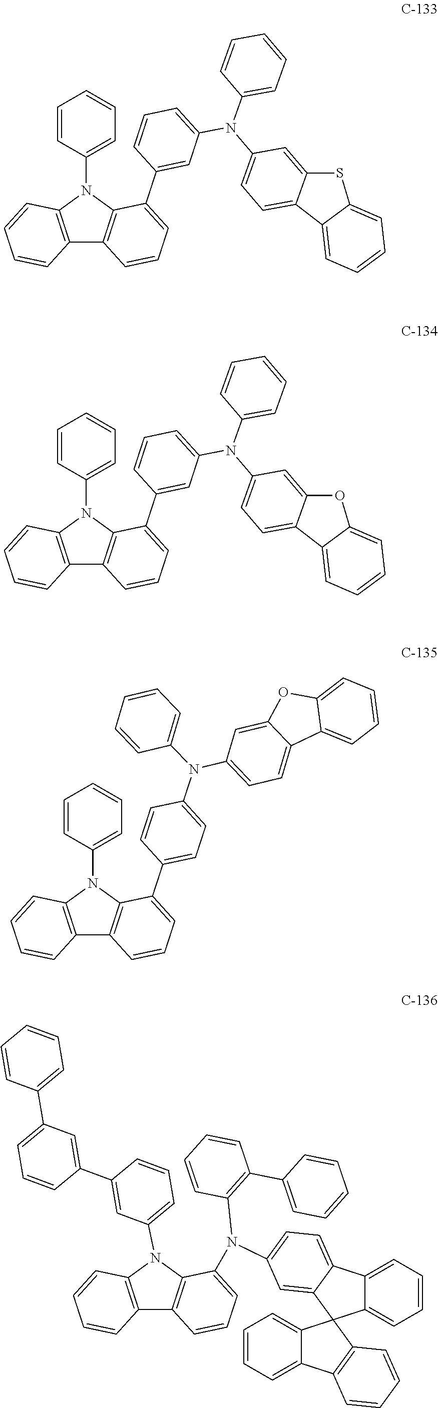 [SMILES: c1ccc(N(c2cccc(-c3cccc4c5ccccc5n(-c5ccccc5)c34)c2)c2ccc3c(c2)sc2ccccc23)cc1 ; c1ccc(N(c2cccc(-c3cccc4c5ccccc5n(-c5ccccc5)c34)c2)c2ccc3c(c2)oc2ccccc23)cc1 ; c1ccc(N(c2ccc(-c3cccc4c5ccccc5n(-c5ccccc5)c34)cc2)c2ccc3c(c2)oc2ccccc23)cc1 ; c1ccc(-c2cccc(-c3cccc(-n4c5ccccc5c5cccc(N(c6ccc7c(c6)C6(c8ccccc8-c8ccccc86)c6ccccc6-7)c6ccccc6-c6ccccc6)c54)c3)c2)cc1]

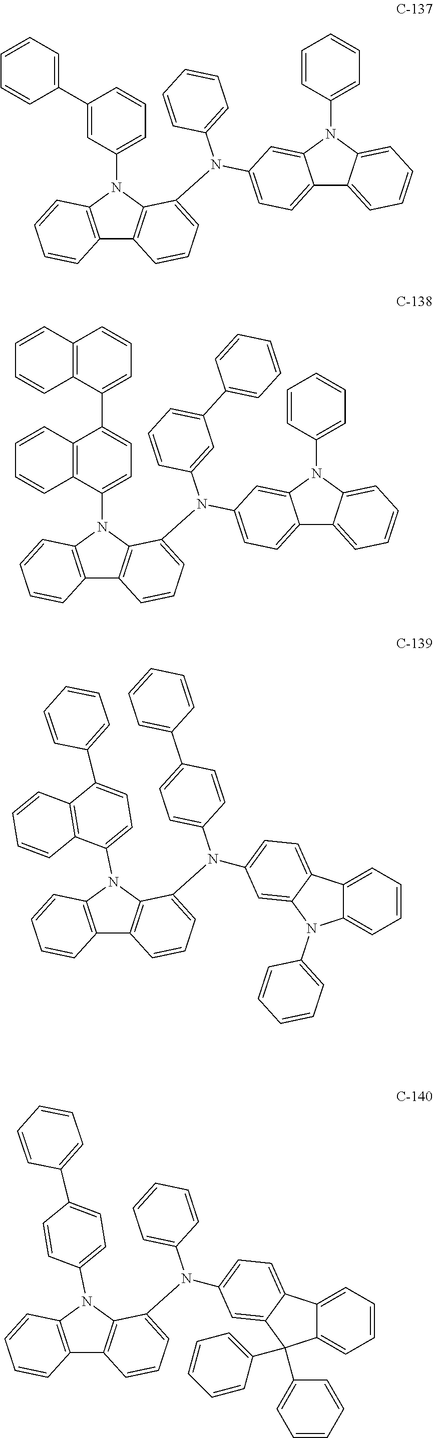 [SMILES: c1ccc(-c2cccc(-n3c4ccccc4c4cccc(N(c5ccccc5)c5ccc6c7ccccc7n(-c7ccccc7)c6c5)c43)c2)cc1 ; c1ccc(-c2cccc(N(c3ccc4c5ccccc5n(-c5ccccc5)c4c3)c3cccc4c5ccccc5n(-c5ccc(-c6cccc7ccccc67)c6ccccc56)c34)c2)cc1 ; c1ccc(-c2ccc(N(c3ccc4c5ccccc5n(-c5ccccc5)c4c3)c3cccc4c5ccccc5n(-c5ccc(-c6ccccc6)c6ccccc56)c34)cc2)cc1 ; c1ccc(-c2ccc(-n3c4ccccc4c4cccc(N(c5ccccc5)c5ccc6c(c5)C(c5ccccc5)(c5ccccc5)c5ccccc5-6)c43)cc2)cc1]

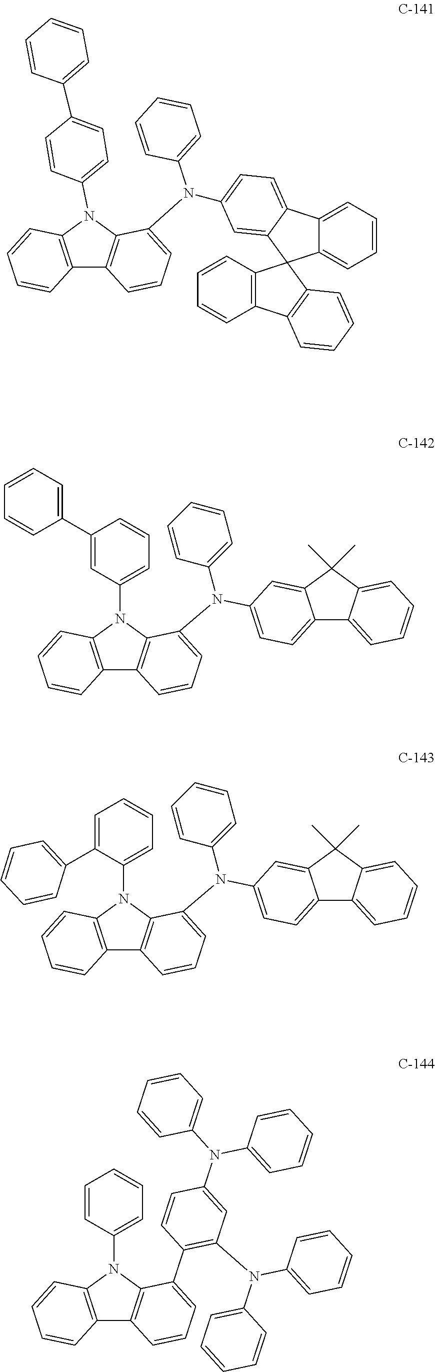 [SMILES: c1ccc(-c2ccc(-n3c4ccccc4c4cccc(N(c5ccccc5)c5ccc6c(c5)C5(c7ccccc7-c7ccccc75)c5ccccc5-6)c43)cc2)cc1 ; CC1(C)c2ccccc2-c2ccc(N(c3ccccc3)c3cccc4c5ccccc5n(-c5cccc(-c6ccccc6)c5)c34)cc21 ; CC1(C)c2ccccc2-c2ccc(N(c3ccccc3)c3cccc4c5ccccc5n(-c5ccccc5-c5ccccc5)c34)cc21 ; c1ccc(N(c2ccccc2)c2ccc(-c3cccc4c5ccccc5n(-c5ccccc5)c34)c(N(c3ccccc3)c3ccccc3)c2)cc1]

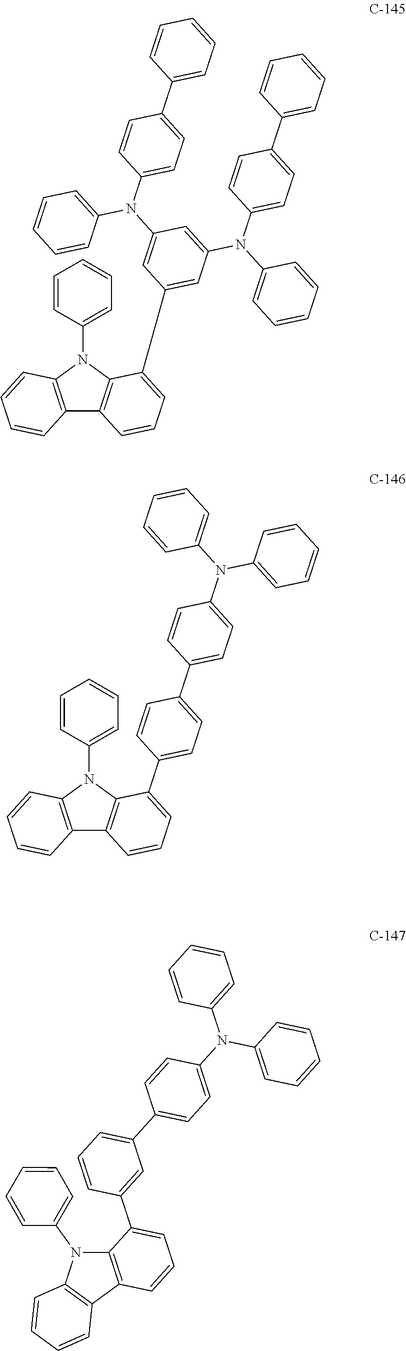 [SMILES: c1ccc(-c2ccc(N(c3ccccc3)c3cc(-c4cccc5c6ccccc6n(-c6ccccc6)c45)cc(N(c4ccccc4)c4ccc(-c5ccccc5)cc4)c3)cc2)cc1 ; c1ccc(N(c2ccccc2)c2ccc(-c3ccc(-c4cccc5c6ccccc6n(-c6ccccc6)c45)cc3)cc2)cc1 ; c1ccc(N(c2ccccc2)c2ccc(-c3cccc(-c4cccc5c6ccccc6n(-c6ccccc6)c45)c3)cc2)cc1]

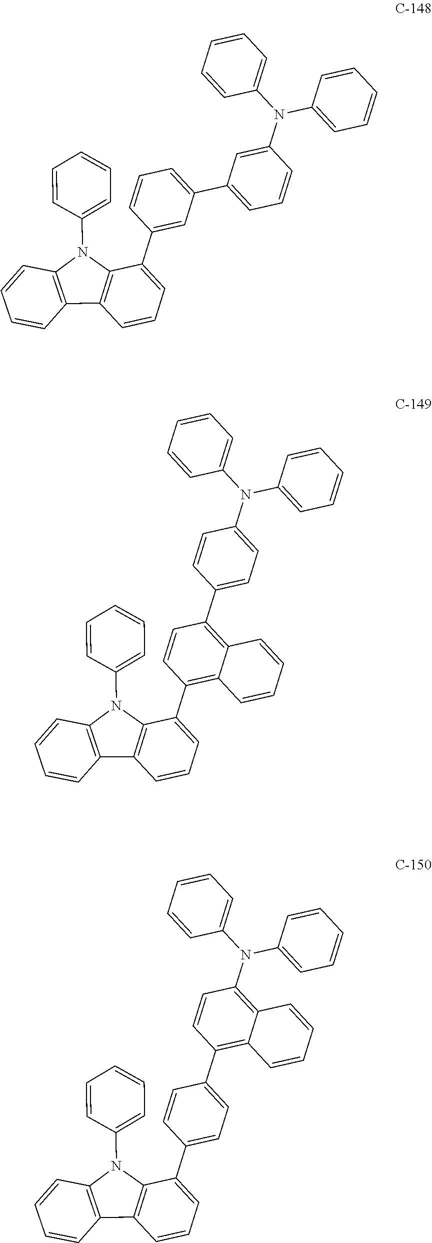 [SMILES: c1ccc(N(c2ccccc2)c2cccc(-c3cccc(-c4cccc5c6ccccc6n(-c6ccccc6)c45)c3)c2)cc1 ; c1ccc(N(c2ccccc2)c2ccc(-c3ccc(-c4cccc5c6ccccc6n(-c6ccccc6)c45)c4ccccc34)cc2)cc1 ; c1ccc(N(c2ccccc2)c2ccc(-c3ccc(-c4cccc5c6ccccc6n(-c6ccccc6)c45)cc3)c3ccccc23)cc1]

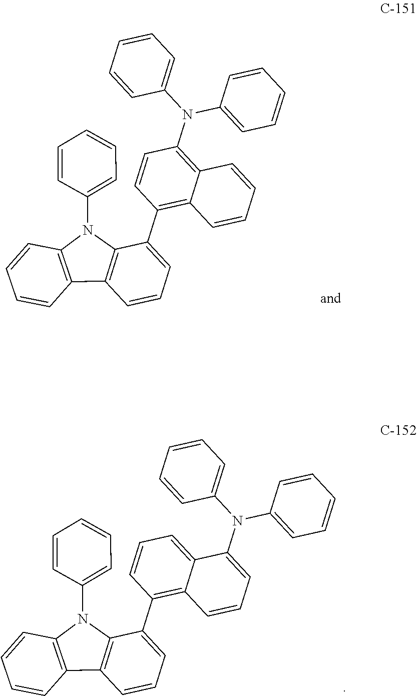 [SMILES: c1ccc(N(c2ccccc2)c2ccc(-c3cccc4c5ccccc5n(-c5ccccc5)c34)c3ccccc23)cc1 ; c1ccc(N(c2ccccc2)c2cccc3c(-c4cccc5c6ccccc6n(-c6ccccc6)c45)cccc23)cc1]

7. An organic electroluminescent device comprising the organic electroluminescent compound according to claim 1.

8. The organic electroluminescent device according to claim 7, wherein the organic electroluminescent compound is contained in a hole transport layer and/or a light-emitting layer.
